(19) United States
(12) Patent Application Publication
(10) Pub. No.: US 2024/0226045 A9
(43) Pub. Date: Jul. 11, 2024

(54) RUMEN-PROTECTED COMPOSITIONS AND METHODS OF MAKING THE SAME

(71) Applicant: Balchem Corporation, Montvale, NJ (US)

(72) Inventor: Gideon Nyabuti Oenga, Middletown, NY (US)

(21) Appl. No.: 18/383,641

(22) Filed: Oct. 25, 2023

Related U.S. Application Data
(60) Provisional application No. 63/419,057, filed on Oct. 25, 2022.

(51) Int. Cl. A61K 31/198; A61K 9/48; A61K 31/14; A61K 31/4172; A61K 47/12; A61K 47/24; A61K 47/26; A61K 47/36

(52) U.S. Cl. CPC A61K 31/198; A61K 9/485; A61K 9/4858; A61K 9/4875; A61K 9/4891; A61K 31/14; A61K 31/4172; A61K 47/12; A61K 47/24; A61K 47/26; A61K 47/36

(57) ABSTRACT

Provided herein are rumen-protected compositions. The compositions include coated rumen-protected compositions which comprise a coating and a bioactive core comprising a physiologically active ingredient. The coating includes a chitosan organic salt, an emulsifier, and a fatty acid source. Further provided are rumen-protected pellet compositions, which may optionally comprise a coating. The pellet compositions include a bioactive core comprising a physiologically active ingredient and a chitosan organic salt. Further provided are methods of making the compositions and methods of delivering methionine to an animal.

### CROSS-REFERENCE TO RELATED APPLICATIONS

This application claims priority to U.S. Provisional Application No. 63/419,057 entitled “RUMEN PROTECTED COMPOSITIONS AND METHODS OF MAKING THE SAME” filed Oct. 25, 2022, the entire contents of which are incorporated by reference herein.

### FIELD

The present application is directed to rumen-protected compositions, and thus relates to animal health and nutrition.

### BACKGROUND

Many commercially available rumen-protected nutrients are coated with lipid formulations that primarily rely on enzymatic digestion and physical breakdown of the coating for nutrient release. This rate of release is entirely based on coating thickness and is often highly variable and unpredictable. Various other materials including proteins, polysaccharides, and synthetic polymers can be used for the encapsulation of ingredients in the food industry. However, as the enzymatic activity of ruminal microorganisms vigorously destroys many of these coating agents, only a limited number have been applied to the ruminant feed industry.

Accordingly, there remains a need in the art for improved compositions for ruminants that have reliable and predictable performance attributes, such as rumen protection and bioavailability, but limited coating material.

### SUMMARY OF THE DISCLOSURE

Provided herein are rumen-protected compositions that provide high levels of total bioavailability of a physiologically active ingredient. The compositions generally comprise a bioactive core comprising a physiologically active ingredient. The bioactive core is surrounded by at least one coating layer. The at least one coating layer comprises a chitosan organic salt, an emulsifier, and a fatty acid source. The coating is generally present in an amount of at least 10% by dry weight of the composition and the ratio of the chitosan organic salt to the emulsifier in the coating layer is less than about 2.5:1, less than about 2:1, or less than about 1:1 by weight. In some embodiments, the at least one coating layer does not include a fat.

In some embodiments, the physiologically active ingredient is selected from the group consisting of methionine, lysine, histidine, choline, and any combination thereof. In some embodiments, the physiologically active ingredient comprises a combination of methionine and lysine. In some embodiments, the physiologically active ingredient comprises a combination of methionine and choline. In some embodiments, the physiologically active ingredient comprises a combination of methionine, lysine, and choline. In some embodiments, the physiologically active ingredient comprises a combination of methionine, histidine, and choline.

In some embodiments, the chitosan organic salt is selected from the group consisting of chitosan acetate, chitosan glutamate, chitosan thiaminate, chitosan aspartate, chitosan nicotinate, and chitosan citrate. In some embodiments, the chitosan organic salt is chitosan acetate.

In some embodiments, the emulsifier is selected from the group consisting of lecithin, monoglycerides, diglycerides, polyglycerol esters, propylene glycol esters, gums, waxes, phosphates, cellulose derivatives, and combinations thereof. In some embodiments, the emulsifier is lecithin.

In some embodiments, the fatty acid source is selected from C12 to C22 saturated fats, C12 to C22 unsaturated fats, C10 to C22 hydrogenated triglycerides, and natural hydrogenated fats.

In some embodiments, the physiologically active ingredient has a total bioavailability of at least about 50%, at least about 60%, at least about 70%, at least about 80%, or at least about 90%. In some embodiments, the physiologically active ingredient has a rumen bypass rate of at least about 50%, at least about 60%, at least about 70% or at least about 80%.

In some embodiments, the rumen-protected composition further comprises a sub-coating. In some embodiments, the sub-coating comprises an oil selected from the group consisting of soybean oil, palm oil, rapeseed oil, cotton oil, linseed oil, castor oil, and other hydrophobic plant-based oils known in the art and combinations thereof. In some embodiments, the sub-coating comprises soybean oil and palm oil.

Further provided herein are rumen-protected pellet compositions that provide high levels of total bioavailability of a physiologically active ingredient. The bioactive core generally comprises a physiologically active ingredient and a chitosan organic salt dispersed throughout the physiologically active ingredient. In some embodiments, the physiologically active ingredient is selected from the group consisting of methionine, lysine, histidine, choline, and any combination thereof.

In some embodiments, the chitosan organic salt is selected from the group consisting of chitosan acetate, chitosan glutamate, chitosan thiaminate, chitosan aspartate, chitosan nicotinate, and chitosan citrate. In some embodiments, the chitosan organic salt is chitosan acetate.

In some embodiments the pellet compositions further comprise an emulsifier. The emulsifier is selected from the group consisting of lecithin, monoglycerides, diglycerides, polyglycerol esters, propylene glycol esters, gums, waxes, phosphates, cellulose derivatives, and combinations thereof. In some embodiments, the emulsifier is lecithin.

In some embodiments the pellet compositions further comprise a fatty acid source. In some embodiments, the fatty acid source is selected from C12 to C22 saturated fats, C12 to C22 unsaturated fats, C10 to C22 hydrogenated triglycerides, and natural hydrogenated fats.

### BRIEF DESCRIPTION OF THE FIGURES

FIG. 1A shows a photograph of two exemplary chitosan acetate films. FIG. 1B shows the films after being submerged in buffer solutions having a pH of 2.0 (left) and a pH of 7.0 (right) at 39° C. for two hours.

FIGS. 2A-2F show photographs of exemplary chitosan and chitosan acetate coating compositions. FIG. 2A shows a chitosan coating composition prior to exposure to a pH 2 buffer and a pH 7 buffer. FIG. 2B shows the chitosan shell composition dissolved in a pH 2 buffer (left) and a pH 7 buffer (right). FIG. 2C shows a chitosan acetate coating composition prior to exposure to a pH 2 buffer and a pH 7 buffer. FIG. 2D shows a chitosan acetate coating composition after dissolution in a pH 2 buffer (left) and a pH 7 buffer (right). FIG. 2E shows a chitosan coating composition wherein a drop of iodine has been placed on the coating composition. FIG. 2F shows a chitosan acetate coating composition wherein a drop of iodine has been placed on the coating composition.

FIG. 3A shows chitosan exposed to a pH 2.0 buffer and a pH 7.0 buffer at 39° C. for 20 hours (top) and after drying (bottom). FIG. 3B shows a mixture of chitosan and lecithin exposed to a pH 2.0 buffer and a pH 7.0 buffer at 39° C. for 20 hours (top) and after drying (bottom). FIG. 3C shows lecithin as a dry powder (top) and after exposing to a pH 2.0 buffer and a pH 7.0 buffer at 39° C. for 20 hours (bottom).

FIG. 4A shows exemplary capsule formulations of the present disclosure. FIG. 3B shows a cross-section of an exemplary capsule formulation of the present disclosure. The coating in FIG. 4B was dyed with iodine.

FIGS. 5A-5F show photographs of rumen-protected compositions made with coatings comprising chitosan and chitosan acetate. FIGS. 5G-5H show results for rumen stability and bioavailability for rumen-protected compositions comprising chitosan (FIG. 5G) and for rumen-protected compositions comprising chitosan acetate (FIG. 5H).

FIG. 6 shows a schematic representation of the model used to derive amino acid entry rates. “T/O” stands for “turnover”. An asterisk denotes isotopic labelling.

FIG. 7 shows plasma methionine concentrations in cows that were abomasally supplied with either a rumen-protected methionine product (RP-MET) or an unprotected methionine product (Unprotected MET).

FIG. 8 shows the abomasal pulse dose trial results for compositions of the present disclosure.

FIG. 9 shows a photograph of the pellet compositions of the present disclosure.

FIG. 10 shows the rumen stability of the pellet compositions of the present disclosure as compared with pellets containing methionine alone.

FIG. 11A shows an image of histidine crystals precoated with a lipid for preparing compositions of the present disclosure. FIG. 11B shows an image of coated histidine compositions of the present disclosure. FIG. 11C shows an image of uncoated histidine crystals.

### DETAILED DESCRIPTION

Provided herein are rumen-protected compositions for delivering a physiologically active ingredient to a ruminant animal. The rumen-protected compositions generally comprise a bioactive core comprising methionine, the core surrounded by at least one coating layer. Generally, the at least one coating layer comprises: a chitosan organic salt, an emulsifier, and a fatty acid source. The inventors have found that the rumen-protected compositions have high degrees of rumen protection and bioavailability. In some embodiments, the rumen-protected compositions may comprise more than one coating layer.

Also provided herein are pellet compositions. The pellet compositions comprise a physiologically active ingredient, a chitosan organic salt, a fatty acid source, and an excipient, and optionally, a coating. Surprisingly, the inventors have found that the pelleted compositions show increased rumen protection and bioavailability with or without a coating layer.

### I. Coated Rumen-Protected Compositions

The present disclosure provides compositions comprising a bioactive core and at least one coating surrounding the core, wherein the composition has high rumen stability and, in preferred embodiments, high bioavailability.

### (A) Bioactive Core

The compositions disclosed may have a bioactive core comprising a physiologically active ingredient, such as methionine, lysine, histidine, choline, and combinations and derivatives thereof. The bioactive core may be an extruded bioactive core, a particulate bioactive core, a granulated bioactive core, and the like. The shape of the bioactive core may be regular or irregular. Typically, the longest measurement of the bioactive core (e.g., length, width, height, diameter) is about 10 mm or less, for instance ≤10 mm, ≤9 mm, ≤8 mm, ≤7 mm, ≤6 mm, ≤5 mm, or a measurement expressed as a range (e.g., about 1 mm to about 10 mm, about 1 mm to about 7 mm, about 1 mm to about 8 mm, about 1 mm to about 7 mm, about 1 mm to about 6 mm, about 1 mm to about 5 mm, about 1 mm to about 4 mm, about 1 mm to about 3 mm, about 1 mm to about 2 mm, etc.).

The bioactive core may be an extruded bioactive core. An extruded bioactive core may have a shape that is cylindrical or irregular. The edges of the shape may be even or uneven. For instance, in some examples, the bioactive core edges may be processed by slightly ‘grinding’ the edges after extrusion to avoid weak and difficult-to-coat spots. In various embodiments, the diameter of a cylindrical shaped bioactive core may range from about 1 mm to about 4 mm, about 1 mm to about 3 mm, or about 1.6 mm to about 2.4 mm. In a specific embodiment, the diameter of a cylindrical shaped bioactive core may be about 2 mm. In further embodiments, the length of a cylindrical shaped bioactive core may range from about 1 mm to about 10 mm, about 1 mm to about 9 mm, about 1 mm to about 8 mm, about 1 mm to about 7 mm, about 1 mm to about 6 mm, about 1.6 mm to about 6.5 mm, about 2 mm to about 6 mm, or about 2 mm to about 4 mm. In specific embodiment, the length of a cylindrically shaped bioactive core may be about 3.0 mm. In another specific embodiment, the length of a cylindrically shaped bioactive core may be about 5.0 mm.

The bulk density of the bioactive core may vary. In embodiments where the bioactive core is to be fluidized for coating, the density is such that the core is dense enough to be fluidized and compact enough to withstand breaking apart during the air flow cycles. In various embodiments, the bulk density of a bioactive core may range from about 0.6 g per cc to about 0.9 g per cc.

### (i) Physiologically Active Ingredient

### (a) Methionine

In some embodiments, the physiologically active ingredient may include methionine. The methionine may be L-methionine, D-methionine, or a combination thereof. The methionine may be in the form of a methionine derivative, such as a methionine salt, solvate, ester, hydrate, etc., or any combination thereof. As a non-limiting example, the methionine may include a combination of L-methionine and D-methionine. As used herein, amounts of methionine refer to the amount of the methionine compound in the composition, including any salt or other derivatized form. For example, a composition including “50% methionine” may refer to either 50% methionine molecule or 50% methionine salt.

In general, the amount of methionine in the bioactive core may range from about 30% to about 99.9% by weight of the uncoated bioactive core. In various embodiments, the amount of methionine present in the bioactive core may be at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, or at least about 95 wt % of the bioactive core. In another embodiment, the bioactive core may comprise about 70 wt % to about 90 wt %, about 75 wt % to about 90 wt %, about 80 wt % to about 90 wt %, or about 85 wt % methionine by weight of the uncoated bioactive core.

In general, the amount of methionine in the bioactive core may range from about 30% to about 85% by weight of the coated composition. In various embodiments, the amount of methionine present in the bioactive core may be at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the coated composition. In another embodiment, the coated composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % methionine by weight of the coated composition.

### (b) Lysine

The physiologically active ingredient may include lysine. The lysine may be L-lysine, D-lysine, or any combination thereof. The lysine may be in the form of a lysine derivative, such as a lysine salt, solvate, ester, hydrate, etc., or any combination thereof. In some non-limiting examples, the lysine may be present in the form of lysine HCl, lysine sulfate, lysine carbonate, or a combination thereof. As a non-limiting example, the lysine may include a combination of lysine sulfate and lysine carbonate. As used herein, amounts of lysine refer to the amount of the lysine compound in the composition, including any salt or other derivatized form. For example, a composition including “50% lysine” may refer to either 50% lysine molecule or 50% lysine HCl.

In general, the amount of lysine in the bioactive core may range from about 30% to about 99.9% by weight of the uncoated bioactive core. In various embodiments, the amount of lysine present in the bioactive core may be at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, or at least about 95 wt % of the bioactive core. In another embodiment, the bioactive core may comprise about 70 wt % to about 99 wt %, about 75 wt % to about 95 wt %, about 80 wt % to about 90 wt %, or about 85 wt % lysine by weight of the uncoated bioactive core. In an exemplary embodiment, the amount of lysine in the bioactive core may be about 99% of the uncoated bioactive core.

In general, the amount of lysine in the bioactive core may range from about 30% to about 85% by weight of the coated composition. In various embodiments, the amount of lysine present in the bioactive core may be at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the coated composition. In another embodiment, the coated composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % lysine by weight of the coated composition. In an exemplary embodiment, the amount of lysine in the bioactive core may range from about 60 wt % to about 80 wt % by weight of the coated composition.

### (c) Methionine and Lysine

In some embodiments, the physiologically active ingredient may include a combination of methionine and lysine. The lysine may serve to provide stability to the methionine. The methionine may be present in any of the amounts described in Section (1)(A)(i)(a) above. In these embodiments, the lysine may be present in an amount of about less than 10% by weight of the uncoated bioactive core; for example, less than 10 wt %, less than 9 wt %, less than 8 wt %, less than 7 wt %, less than 6 wt %, or less than 5 wt %. In another embodiment, the lysine may be present in an amount from about 1% to about 10% by weight of the uncoated bioactive core, for example, from about 1 wt % to about 2 wt %, about 1 wt % to about 3 wt %, about 1 wt % to about 4 wt %, about 1 wt % to about 5 wt %, about 1 wt % to about 6 wt %, about 1 wt % to about 7 wt %, about 1 wt % to about 8 wt %, about 1 wt % to about 9 wt %, about 2 wt % to about 10 wt %, about 3 wt % to about 10 wt %, about 4 wt % to about 10 wt %, about 5 wt % to about 10 wt %, about 6 wt % to about 10 wt %, about 7 wt % to about 10 wt %, about 8 wt % to about 10 wt %, or about 9 wt % to about 10 wt %.

The lysine may be present in an amount of about less than 10% by weight of the coated composition; for example, less than 10 wt %, less than 9 wt %, less than 8 wt %, less than 7 wt %, less than 6 wt %, or less than 5 wt %. In another embodiment, the lysine may be present in an amount from about 1% to about 10% by weight of the coated composition, for example, from about 1 wt % to about 2 wt %, about 1 wt % to about 3 wt %, about 1 wt % to about 4 wt %, about 1 wt % to about 5 wt %, about 1 wt % to about 6 wt %, about 1 wt % to about 7 wt %, about 1 wt % to about 8 wt %, about 1 wt % to about 9 wt %, about 2 wt % to about 10 wt %, about 3 wt % to about 10 wt %, about 4 wt % to about 10 wt %, about 5 wt % to about 10 wt %, about 6 wt % to about 10 wt %, about 7 wt % to about 10 wt %, about 8 wt % to about 10 wt %, or about 9 wt % to about 10 wt %.

### (d) Histidine

The physiologically active ingredient may include histidine. The histidine may be L-histidine, D-histidine, or a combination thereof. The histidine may be in the form of a histidine derivative, such as a histidine salt, solvate, ester, hydrate etc. In some non-limiting examples, the histidine may be present in the form of histidine hydrochloride or histidine hydrochloride monohydrate, or a combination thereof. As used herein, amounts of histidine refer to the amount of the histidine compound in the composition, including any salt or other derivatized form. For example, a composition including “50% histidine” may refer to either 50% histidine molecule or 50% histidine hydrochloride monohydrate.

In general, the amount of histidine in the bioactive core may range from about 30% to about 99.9% by weight of the uncoated bioactive core. In various embodiments, the amount of histidine present in the bioactive core may be at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, or at least about 95 wt % of the bioactive core. In another embodiment, the bioactive core may comprise about 70 wt % to about 90 wt %, about 75 wt % to about 90 wt %, about 80 wt % to about 90 wt %, or about 85 wt % histidine by weight of the uncoated bioactive core. In an exemplary embodiment, the amount of histidine in the bioactive core is about 99.97% by weight of the uncoated bioactive core.

In general, the amount of histidine in the bioactive core may range from about 30% to about 85% by weight of the coated composition. In various embodiments, the amount of histidine present in the bioactive core may be at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the coated composition. In another embodiment, the coated composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % histidine by weight of the coated composition. In an exemplary embodiment, the amount of histidine present in the bioactive core is from about 50 wt % to about 80 wt % by weight of the coated composition.

### (e) Choline

The physiologically active ingredient may include choline. The choline may be in the form of a choline derivative, such as a choline salt, solvate, ester, hydrate, etc., or any combination thereof. In some non-limiting examples, the choline may be present in the form of choline bitartrate, choline chloride, choline dihydrogen citrate, choline salicylate, choline phosphate, choline bicarbonate, choline magnesium trisalicylate, phosphatidylcholine, choline alginate, choline lactate, choline malate, choline aspartate, choline glutamate, or any combination thereof. As a non-limiting example, the choline may include a combination of choline bitartrate and choline chloride. As used herein, amounts of choline refer to the amount of the choline compound in the composition, including any salt or other derivatized form. For example, a composition including “50% choline” may refer to either 50% choline molecule or 50% choline chloride.

In general, the amount of choline in the bioactive core may range from about 30% to about 99.9% by weight of the uncoated bioactive core. In various embodiments, the amount of choline present in the bioactive core may be at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, or at least about 95 wt % of the bioactive core. In another embodiment, the bioactive core may comprise about 70 wt % to about 90 wt %, about 75 wt % to about 90 wt %, about 80 wt % to about 90 wt %, or about 85 wt % choline by weight of the uncoated bioactive core. In an exemplary embodiment, the amount of choline in the bioactive core is about 99.97% by weight of the uncoated bioactive core.

In general, the amount of choline in the bioactive core may range from about 30% to about 85% by weight of the coated composition. In various embodiments, the amount of choline present in the bioactive core may be at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the coated composition. In another embodiment, the coated composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % choline by weight of the coated composition. In an exemplary embodiment, the amount of choline present in the bioactive core is from about 50 wt % to about 80 wt % by weight of the coated composition.

### (f) Other Combinations

In some embodiments, the physiologically active ingredient may include a combination of choline and methionine. The amount of choline in the bioactive core may range from about 60% to about 70% by weight of the uncoated bioactive core. For example, the bioactive core may comprise choline in an amount from about 60 wt % to about 62.5 wt %, about 60 wt % to about 65 wt %, about 60 wt % to about 67.5 wt %, about 60 wt % to about 70 wt %, about 62.5 wt % to about 65 wt %, about 62.5 wt % to about 67.5 wt %, about 62.5 wt % to about 70 wt %, about 65 wt % to about 67.5 wt %, about 65 wt % to about 70 wt %, or about 67.5 wt % to about 70 wt % by weight of the bioactive core. The amount of methionine in the bioactive core may range from about 15% to about 20% by weight of the uncoated bioactive core. For example, the bioactive core may comprise methionine in an amount from about 15 wt % to about 17.5 wt %, about 15 wt % to about 20 wt %, or about 17.5 wt % to about 20 wt % by weight of the uncoated bioactive core.

In some embodiments, the physiologically active ingredient may include a combination of choline, histidine and methionine. The amount of choline in the bioactive core may range from about 40% to about 60% by weight of the uncoated bioactive core. For example, the bioactive core may comprise choline in an amount from about 40 wt % to about 45 wt %, about 40 wt % to about 50 wt %, about 40 wt % to about 55 wt %, about 40 wt % to about 60 wt %, about 45 wt % to about 50 wt %, about 45 wt % to about 55 wt %, about 45 wt % to about 60 wt %, about 50 wt % to about 55 wt %, about 50 wt % to about 60 wt %, or about 55 wt % to about 60 wt % by weight of the bioactive core. The amount of histidine in the bioactive core may range from about 10% to about 30% by weight of the uncoated bioactive core. For example, the bioactive core may comprise histidine in an amount from about 10 wt % to about 15 wt %, about 10 wt % to about 20 wt %, about 10 wt % to about 25 wt %, about 10 wt % to about 30 wt %, about 15 wt % to about 20 wt %, about 15 wt % to about 25 wt %, about 15 wt % to about 30 wt %, about 20 wt % to about 25 wt %, about 20 wt % to about 30 wt %, or about 25 wt % to about 30 wt % by weight of the bioactive core. The amount of methionine in the bioactive core may range from about 5% to about 10% by weight of the uncoated bioactive core. For example, the bioactive core may comprise methionine in an amount from about 5 wt % to about 7.5 wt %, about 5 wt % to about 10 wt %, or about 7.5 wt % to about 10 wt % by weight of the uncoated bioactive core.

In some embodiments, the physiologically active ingredient may include a combination of choline, methionine and lysine. The amount of choline in the bioactive core may range from about 40% to about 60% by weight of the uncoated bioactive core. For example, the bioactive core may comprise choline in an amount from about 40 wt % to about 45 wt %, about 40 wt % to about 50 wt %, about 40 wt % to about 55 wt %, about 40 wt % to about 60 wt %, about 45 wt % to about 50 wt %, about 45 wt % to about 55 wt %, about 45 wt % to about 60 wt %, about 50 wt % to about 55 wt %, about 50 wt % to about 60 wt %, or about 55 wt % to about 60 wt % by weight of the bioactive core. The amount of methionine in the bioactive core may range from about 10% to about 30% by weight of the uncoated bioactive core. For example, the bioactive core may comprise methionine in an amount from about 10 wt % to about 15 wt %, about 10 wt % to about 20 wt %, about 10 wt % to about 25 wt %, about 10 wt % to about 30 wt %, about 15 wt % to about 20 wt %, about 15 wt % to about 25 wt %, about 15 wt % to about 30 wt %, about 20 wt % to about 25 wt %, about 20 wt % to about 30 wt %, or about 25 wt % to about 30 wt % by weight of the bioactive core. The amount of lysine in the bioactive core may range from about 5% to about 10% by weight of the uncoated bioactive core. For example, the bioactive core may comprise lysine in an amount from about 5 wt % to about 7.5 wt %, about 5 wt % to about 10 wt %, or about 7.5 wt % to about 10 wt % by weight of the uncoated bioactive core.

### (ii) Fatty Acid Source

The bioactive core may further comprise a fatty acid source. The fatty acid source of the core may include a short-chain fatty acid (e.g., C₁-C₅), a medium-chain fatty acid (e.g., C₆-C₁₂), a long-chain fatty acid (e.g., C₁₃-C₂₁), a very long-chain fatty acid (e.g., C₂₂ or more), or combinations thereof. In preferred embodiments, the fatty acid source is selected from C₁₂-C₂₂ saturated fats, C₁₂-C₂₂ unsaturated fats, C₁₀-C₂₂ hydrogenated triglycerides, C₁₀-C₂₂ hydrogenated mono-diglycerides, natural hydrogenated fats such as hydrogenated vegetable oils, and combinations thereof. In some examples, the fatty acid source may comprise acetic acid, arachidic acid, behenic acid, butyric acid, capric acid, caprylic acid, cerotic acid, lauric acid, lignoceric acid, myristic acid, palmitic acid, propionic acid, stearic acid, and combinations thereof. In a preferred embodiment, the fatty acid source comprises palmitic acid, stearic acid, or a combination thereof.

The fatty acid source may be present in the uncoated bioactive core in an amount of about 5 wt % to about 15 wt %. For example, the fatty acid source may be present in the bioactive core in an amount of about 5 wt %, about 6 wt %, about 7 wt %, about 8 wt %, about 9 wt %, about 10 wt %, about 11 wt %, about 12 wt %, about 13 wt %, about 14 wt %, or about 15 wt %. In some embodiments, the fatty acid source may be present in the bioactive core in an amount of about 6 wt % to about 14 wt %, about 7 wt % to about 13 wt %, about 8 wt % to about 12 wt %, about 5 wt % to about 10 wt % or about 10 wt % to about 15 wt %. For example, the fatty acid source may be present in the bioactive core in an amount of about 7%, about 8%, about 9%, about 10%, about 11%, about 12%, or about 13%.

The fatty acid source may be present in the bioactive core of the coated composition in an amount of about 5% to about 10% by weight of the coated composition. For example, the fatty acid source may be present in the bioactive core of the coated composition in an amount of about 5%, about 6%, about 7%, about 8%, about 9%, or about 10% by weight of the coated composition.

### (iii) Excipients

The bioactive core may further comprise one or more excipients. A variety of excipients may be included in the coated rumen-protected compositions. Suitable excipients include fillers, binders, pH regulating agents, disintegrants, dispersing agents, preservatives, lubricants, coloring agents, flavoring agents, taste masking agents, film forming agents, stabilizers, or combinations thereof. In general, the excipient is a grade suitable for use in a nutritional composition. In some embodiments, the bioactive core does not comprise any excipients.

The excipient may comprise at least one filler. Non-limiting examples of suitable fillers (also called diluents) include cellulose, microcrystalline cellulose, cellulose ethers (e.g., ethyl cellulose, methyl cellulose, hydroxypropyl cellulose, hydroxypropyl methylcellulose, sodium carboxymethylcellulose, etc.), cellulose esters (i.e., cellulose acetate, cellulose butyrate, and mixtures thereof), starches (e.g., corn starch, rice starch, potato starch, tapioca starch, and the like), modified starches, pregelatinized starches, phosphated starches, starch-lactose, starch-calcium carbonate, sodium starch glycolate, saccharides (e.g., glucose, fructose, sucrose, lactose, xylose, and so forth), lactitol, mannitol, maltitol, sorbitol, xylitol, maltodextrin, trehalose, inorganic materials (e.g., calcium carbonate, calcium sulfate, calcium phosphate, calcium silicate, magnesium carbonate, magnesium oxide, talc, etc.), or combinations thereof.

The excipient may comprise at least one binder. Examples of suitable binders include, without limit, starches (e.g., corn starch, potato starch, wheat starch, rice starch, and the like), pregelatinized starch, hydrolyzed starch, cellulose, microcrystalline cellulose, cellulose derivatives (e.g., methylcellulose, ethylcellulose, hydroxypropylcellulose, hydroxypropylmethylcellulose, sodium carboxymethylcellulose, and the like), saccharides (e.g., sucrose, lactose, and so forth), sugar alcohols (e.g., maltitol, sorbitol, xylitol, polyethylene glycol, and the like), alginates (e.g., alginic acid, alginate, sodium alginate, and so forth), gums (e.g., gum arabic, guar gum, gellan gum, xanthan gum, and the like), pectins, gelatin, C₁₂-C₁₈ fatty acid alcohols, polyvinylpyrrolidone (also called copovidone), polyethylene oxide, polyethylene glycol, polyvinyl alcohols, waxes (e.g., candelilla wax, caranuba wax, beeswax, and so forth), or combinations of any of the foregoing.

The excipient may comprise a pH regulating agent. By way of non-limiting example, pH regulating agents include organic carboxylic acids (e.g., acetic acid, ascorbic acid, citric acid, formic acid, glycolic acid, gluconic acid, lactic acid, malic acid, maleic acid, propionic acid, succinic acid, tartaric acid, etc.) or salts thereof other acids (e.g., hydrochloric acid, boric acid, nitric acid, phosphoric acid, sulfuric acid, etc.), alkali metal or ammonium carbonates, bicarbonates, hydroxides, phosphates, nitrates, and silicates; and organic bases (such as, for example, pyridine, triethylamine (i.e., monoethanol amine), diisopropylethylamine, N methylmorpholine, N,N dimethylaminopyridine).

The excipient may comprise a disintegrant. Examples of suitable disintegrants include, without limit, povidone, crospovidone, croscarmellose sodium, sodium carboxymethylcellulose, carboxymethylcellulose calcium, sodium starch glycolate, cellulose, microcrystalline cellulose, methylcellulose, silicon dioxide (also called colloidal silicone dioxide), alginates (e.g., alginic acid, alginate, sodium alginate, and so forth), clays (e.g., bentonite), calcium carbonate, or combinations thereof.

The excipient may comprise dispersing agent. Suitable dispersing agents include, but are not limited to, starch, alginic acid, polyvinylpyrrolidones, guar gum, kaolin, bentonite, purified wood cellulose, sodium starch glycolate, isamorphous silicate, and microcrystalline cellulose as high HLB emulsifier surfactants.

The excipient may comprise a preservative. Non limiting examples of suitable preservatives include antioxidants (such as, e.g., alpha-tocopherol, ascorbyl palmitate, butylated hydroxyanisole, butylated hydroxytoluene, citric acid, dihydroguaretic acid, potassium ascorbate, potassium sorbate, propylgallate, sodium bisulfate, sodium isoascorbate, sodium metabisulfite, sorbic acid, 4-chloro-2,6-ditertiarybutylphenol, and so forth), antimicrobials (such as, e.g., benzyl alcohol, cetylpyridine chloride, glycerine, parabens, propylene glycol, potassium sorbate, sodium benzoate, sorbic acid, sodium propionate, and the like), or combinations thereof.

The excipient may comprise a lubricant. Examples of suitable lubricants include metal stearate such as magnesium stearate, calcium stearate, zinc stearate, a polyethylene glycol, a poloxamer, colloidal silicon dioxide, glyceryl behenate, light mineral oil, hydrogenated vegetable oils, magnesium lauryl sulfate, magnesium trisilicate, polyoxyethylene monostearate, sodium stearoyl fumarate, sodium stearyl fumarate, sodium benzoate, sodium lauryl sulfate, stearic acid, sterotex, talc, or combinations thereof.

The excipient may comprise a color additive. Suitable color additives include, but are not limited to, food, drug and cosmetic colors (FD&C), drug and cosmetic colors (D&C), or external drug and cosmetic colors (Ext. D&C). drug and cosmetic colors (D&C), or external drug and cosmetic colors (Ext. D&C). These colors or dyes, along with their corresponding lakes, and certain natural and derived colorants may be suitable for use in the compositions.

The excipient may comprise a flavoring agent. Flavoring agents may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oils (such as lemon oil, orange oil, grape and grapefruit oil), and fruit essences (such as apple, peach, pear, strawberry, raspberry, cherry, plum, pineapple, and apricot). In still another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; stevia-derived sweeteners; chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof. In still another embodiment, the excipient may include a taste-masking agent.

The excipient may comprise a taste-masking agent. Suitable taste masking agents include cellulose hydroxypropyl ethers (HPC); low-substituted hydroxypropyl ethers (L-HPC); cellulose hydroxypropyl methyl ethers (HPMC); methylcellulose polymers and mixtures thereof; polyvinyl alcohol (PVA); hydroxyethylcelluloses; carboxymethylcelluloses and salts thereof; polyvinyl alcohol and polyethylene glycol co-polymers; monoglycerides or triglycerides; polyethylene glycols; acrylic polymers; mixtures of acrylic polymers with cellulose ethers; cellulose acetate phthalate; or combinations thereof.

The excipient may comprise a stabilizer. Suitable stabilizers include alginates, agar, carrageen, cellulose and cellulose derivatives, guar gum, gelatin, gum Arabic, locust bean gum, pectin, starch, xanthan gum, and combinations thereof.

### (B) Coating

The compositions provided herein contain a coating that fully surrounds the core. The coating comprises chitosan organic salt, an emulsifier, and at least one fatty acid. The coating may be present in the coated composition in an amount of not more than about 50% by dry weight of the coated composition; for example, not more than about 45 wt %, not more than about 40 wt %, not more than about 35 wt %, not more than about 30 wt %, not more than about 25 wt %, not more than about 20 wt %, or not more than about 15 wt %. The coating may be present in an amount of at least about 10% by dry weight of the coated composition. In some embodiments, the coating may be present in an amount of about 10% to about 25% by weight of the coated composition. For instance, the coating may be present in an amount from about 10%, about 11%, about 12%, about 13%, about 14%, about 15% about 16%, about 17%, about 18%, about 19%, about 20%, about 21%, about 22%, about 23%, about 24%, or about 25% by weight of the coated composition. In other embodiments, the coating may be present in an amount from about 10% to about 20% by weight of the coated composition. For instance, the coating may be present in the composition in an amount of about 10%, about 11%, about 12%, about 13%, about 14%, about 15%, about 16%, about 17%, about 18%, about 19%, or about 20%. In preferred embodiments, the coating may be present in an amount from about 15% to about 20%. For example, the coating may be present in an amount of about 15%, about 16%, about 17%, about 18%, about 19%, or about 20%.

### (i) Chitosan Organic Salt

Chitosan is a natural polymer derived from chitin by alkaline conversion. The source of chitin is non-limiting. The degree of deacetylation, the molecular weight, and the percentage of free amine groups can vary among chitosan preparations. Suitable chitosan typically has at least about 80% free amino groups available, is at least about 85% deacetylated, and has a molecular weight of about 100 kDa to about 600 kDa. Chitosan is commercially available in the form of solutions, flakes, fine powders, beads, and fibers, any and all of which can be used. Organic salts of chitosan are also available in such forms and may be produced by combining chitosan with a water-soluble organic acid.

The coating of the coated compositions of the present disclosure comprises a chitosan organic salt. In some embodiments, the chitosan organic salt may be selected from the group consisting of chitosan acetate, chitosan glutamate, chitosan thiaminate, chitosan aspartate, chitosan nicotinate, chitosan citrate, chitosan propionate, chitosan butanoate, chitosan formate, chitosan tartrate, chitosan malate, chitosan oxalate, chitosan ascorbate, and chitosan urate. It was surprisingly found that chitosan alone and inorganic salts of chitosan (e.g., chitosan chloride) were ineffective in producing a pH responsive coating, as the examples described hereinbelow show.

Preferably, the coated compositions of the present disclosure do not include chitosan alone, or an inorganic salt of chitosan. It was surprisingly found that chitosan alone and inorganic salts of chitosan are not pH responsive and thus provide much less rumen protection and total bioavailability as compared to organic salts of chitosan.

The chitosan organic salt may be present in the coated composition (i.e., the bioactive core plus the coating) in an amount no less than about 0.05% by weight. The chitosan organic salt may be present in the coated composition in an amount of about 0.05% by weight to about 3% by weight. For example, the chitosan organic salt may be present in the coated composition in an amount of about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0% by weight. In some embodiments, the chitosan organic salt may be present in the coated composition in an amount from about 0.5% to about 2.5%, or more preferably from about 1.0% to about 2.0%, or even more preferably from about 1.2% to about 1.8% by weight.

The chitosan organic salt may be present in the coating in an amount of about 3% to about 10% on a dry weight basis. For example, the chitosan organic salt may be present in the coating in an amount of about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, or about 10% on a dry weight basis. In some embodiments, the chitosan organic salt may be present in the coating in an amount from about 4% to about 9%, or more preferably from about 6% to about 8% on a dry weight basis.

The chitosan organic salt may comprise an amount of the corresponding organic acid. Preferably, the organic acid content is minimized. In some aspects, the organic acid content of the chitosan organic salt is about 2% by weight or less (i.e., about 2 grams or less of organic acid in every 100 grams of chitosan organic salt). For example, the organic acid content of the chitosan organic salt may be about 2% or less, about 1.5% or less, about 1% or less, about 0.5% or less, about 0.1% or less, or about 0.01% or less by weight. In some examples, the organic acid content of the chitosan organic salt is about 2%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or less than 0.01% by weight.

In a particular example, when the chitosan organic salt is chitosan acetate, the chitosan acetate may comprise acetic acid. Preferably, the acetic acid content of the chitosan acetate is minimized because too much acetic acid in the chitosan acetate may reduce the pH responsiveness of the composition. In some embodiments, the acetic acid content of the chitosan acetate is about 2% by weight or less (i.e., about 2 grams or less of acetic acid in every 100 grams of chitosan acetate). For example, the acetic acid content of the chitosan acetate may be about 2% or less, about 1.5% or less, about 1% or less, about 0.5% or less, about 0.1% or less, or about 0.01% or less by weight. In some examples, the acetic acid content of the chitosan acetate is about 2%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or less than 0.01% by weight.

### (ii) Emulsifier

The coating comprises an emulsifier. Emulsifiers used in food and animal health products are generally known in the art, and may include lecithins, monoglycerides, diglycerides, polyglycerol esters, propylene glycol esters, gums (e.g., gum acacia), phosphates, cellulose derivatives, sorbitan esters, polysorbates, stearoyl lactylate sodium, oleate salts, zein, caseinate salts, epoxidized oils (e.g., epoxidized soybean oil), phthalates, vegetable oil (e.g, avocado oil, olive oil, sesame seed oil), animal-derived oils, and combinations thereof. In preferred embodiments, the emulsifier is selected from the group consisting of lecithin, monoglycerides, diglycerides, polyglycerol esters, propylene glycol esters, gums, waxes, phosphates, cellulose derivatives, and combinations thereof.

Preferably, the emulsifier comprises a lecithin. Lecithins are mixtures of glycerophospholipids including phosphatidylcholine, phosphatidylethanolamine, phosphatidylinositol, phosphatidylserine, and phosphatidic acid. Common sources of lecithin include soybeans, milk, rapeseed, cottonseed, sunflower oil, egg yolk, marine foods, etc. The source of the lecithin is non-limiting. In preferred embodiments, the lecithin comprises deoiled lecithin. Deoiled lecithin contains a higher concentration of phospholipids as compared to non-deoiled lecithin. Generally, the oil is removed from the lecithin by extraction with a solvent, such as acetone.

The emulsifier may be present in the coated composition in an amount of about 0.05% by weight to about 3% by weight of the coated composition. For example, the emulsifier may be present in the coated composition in an amount of about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0% by weight of the coated composition. In some embodiments, the emulsifier may be present in the coated composition in an amount of about 0.5% to about 2.5%, or more preferably about 1.0% to about 2.0%, or even more preferably about 1.2% to about 1.8% by weight of the coated composition.

The emulsifier may be present in the coating in an amount of about 3% to about 10% on a dry weight basis. For example, the emulsifier may be present in the coating in an amount of about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, or about 10% on a dry weight basis. In some embodiments, the emulsifier may be present in the coating in an amount of about 4% to about 9%, or more preferably about 6% to about 8% on a dry weight basis.

It has surprisingly been found that the ratio of the chitosan organic salt to the emulsifier is critical to achieving high total bioavailability of the methionine. Preferably, the ratio of the chitosan organic salt to the emulsifier is less than about 2.5:1 on a dry weight basis. For example, the ratio of the chitosan organic salt to the emulsifier may be less than about 2.5:1, less than about 2.4:1, less than about 2.3:1, less than about 2.2:1, less than about 2.1:1, less than about 2.0:1, less than about 1.9:1, less than about 1.8:1, less than about 1.7:1, less than about 1.6:1, less than about 1.5:1, less than about 1.4:1, less than about 1.3:1, less than about 1.2:1, less than about 1.1:1, or less than about 1.0:1 on a dry weight basis.

### (iii) Fatty Acid Source

The coating further comprises a fatty acid source. The fatty acid source may include a short-chain fatty acid (e.g., C₁-C₅), a medium-chain fatty acid (e.g., C₆-C₁₂), a long-chain fatty acid (e.g., C₁₃-C₂₁), a very long-chain fatty acid (e.g., C₂₂ or more), or combinations thereof. The fatty acid source may comprise a saturated fatty acid or an unsaturated fatty acid, but is preferably a saturated fatty acid. In some examples, the fatty acid source may comprise acetic acid, arachidic acid, behenic acid, butyric acid, capric acid, caprylic acid, cerotic acid, lauric acid, lignoceric acid, myristic acid, palmitic acid, propionic acid, stearic acid, and combinations thereof. In a preferred embodiment, the fatty acid source comprises palmitic acid, stearic acid, or a combination thereof.

The fatty acid source may be present in the coating in an amount from about 75% to about 95% on a dry weight basis. For example, the fatty acid source may be present in the coating in an amount of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95% on a dry weight basis. In some embodiments, the fatty acid source may be present in the coating in an amount from about 80% to about 90% on a dry weight basis. For example, the fatty acid source may be present in the coating in an amount of about 80%, about 81%, about 82%, about 83%, about 84%, about 85%, about 86%, about 87%, about 88%, about 89%, or about 90%.

The fatty acid source may be present in the coated composition (i.e., the coating plus the bioactive core) in an amount from about 5% to about 25% by weight. For example, the fatty acid source may be present in the coated composition in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%. In some embodiments, the fatty acid source may be present in the coated composition in an amount from about 10% to about 20% by weight. For example, the fatty acid source may be present in the coated composition in an amount of about 10%, about 11%, about 12%, about 13%, about 14%, about 15%, about 16%, about 17%, about 18%, about 19%, or about 20% by weight.

In preferred embodiments, the fatty acid source comprises a mixture of stearic acid and palmitic acid, wherein the weight ratio of the stearic acid to the palmitic acid is from about 40:60 to about 60:40. In a preferred embodiment, the weight ratio of the stearic acid to the palmitic acid is about 1:1.

In preferred embodiments, the coating does not include a fat.

### (iv) Excipients

The coating may further include one or more excipients. Suitable excipients include fillers, binders, pH regulating agents, disintegrants, dispersing agents, preservatives, lubricants, coloring agents, flavoring agents, taste masking agents, stabilizers, film-forming agents, or combinations thereof. In general, the excipient is a grade suitable for use in a nutritional composition. In some embodiments, the bioactive core does not comprise any excipients. The excipients may be the same as or different from excipients included in the bioactive core.

The excipient may comprise at least one filler. Non-limiting examples of suitable fillers (also called diluents) include cellulose, microcrystalline cellulose, cellulose ethers (e.g., ethyl cellulose, methyl cellulose, hydroxypropyl cellulose, hydroxypropyl methylcellulose, sodium carboxymethylcellulose, etc.), cellulose esters (i.e., cellulose acetate, cellulose butyrate, and mixtures thereof), starches (e.g., corn starch, rice starch, potato starch, tapioca starch, and the like), modified starches, pregelatinized starches, phosphate starches, starch-lactose, starch-calcium carbonate, sodium starch glycolate, saccharides (e.g., glucose, fructose, sucrose, lactose, xylose, and so forth), lactitol, mannitol, maltitol, sorbitol, xylitol, maltodextrin, trehalose, inorganic materials (e.g., calcium carbonate, calcium sulfate, calcium phosphate, calcium silicate, magnesium carbonate, magnesium oxide, talc, etc.), or combinations thereof.

The excipient may comprise at least one binder. Examples of suitable binders include, without limit, starches (e.g., corn starch, potato starch, wheat starch, rice starch, and the like), pregelatinized starch, hydrolyzed starch, cellulose, microcrystalline cellulose, cellulose derivatives (e.g., methylcellulose, ethylcellulose, hydroxypropylcellulose, hydroxypropylmethylcellulose, sodium carboxymethylcellulose, and the like), saccharides (e.g., sucrose, lactose, and so forth), sugar alcohols (e.g., maltitol, sorbitol, xylitol, polyethylene glycol, and the like), alginates (e.g., alginic acid, alginate, sodium alginate, and so forth), gums (e.g., gum arabic, guar gum, gellan gum, xanthan gum, and the like), pectins, gelatin, C₁₂-C₁₈ fatty acid alcohols, polyvinylpyrrolidone (also called copovidone), polyethylene oxide, polyethylene glycol, polyvinyl alcohols, waxes (e.g., candelilla wax, caranuba wax, beeswax, and so forth), or combinations of any of the foregoing.

The excipient may comprise a pH regulating agent. By way of non-limiting example, pH regulating agents include organic carboxylic acids (e.g., acetic acid, ascorbic acid, citric acid, formic acid, glycolic acid, gluconic acid, lactic acid, malic acid, maleic acid, propionic acid, succinic acid, tartaric acid, etc.) or salts thereof other acids (e.g., hydrochloric acid, boric acid, nitric acid, phosphoric acid, sulfuric acid, etc.), alkali metal or ammonium carbonates, bicarbonates, hydroxides, phosphates, nitrates, and silicates; and organic bases (such as, for example, pyridine, triethylamine (i.e., monoethanol amine), diisopropylethylamine, N methylmorpholine, N,N dimethylaminopyridine).

The excipient may comprise a disintegrant. Examples of suitable disintegrants include, without limit, povidone, crospovidone, croscarmellose sodium, sodium carboxymethylcellulose, carboxymethylcellulose calcium, sodium starch glycolate, cellulose, microcrystalline cellulose, methylcellulose, silicon dioxide (also called colloidal silicone dioxide), alginates (e.g., alginic acid, alginate, sodium alginate, and so forth), clays (e.g., bentonite), or combinations thereof.

The excipient may comprise dispersing agent. Suitable dispersing agents include, but are not limited to, starch, alginic acid, polyvinylpyrrolidones, guar gum, kaolin, bentonite, purified wood cellulose, sodium starch glycolate, isamorphous silicate, and microcrystalline cellulose as high HLB emulsifier surfactants.

The excipient may comprise a preservative. Non limiting examples of suitable preservatives include antioxidants (such as, e.g., alpha-tocopherol, ascorbyl palmitate, butylated hydroxyanisole, butylated hydroxytoluene, citric acid, dihydroguaretic acid, potassium ascorbate, potassium sorbate, propylgallate, sodium bisulfate, sodium isoascorbate, sodium metabisulfite, sorbic acid, 4-chloro-2,6-ditertiarybutylphenol, and so forth), antimicrobials (such as, e.g., benzyl alcohol, cetylpyridine chloride, glycerine, parabens, propylene glycol, potassium sorbate, sodium benzoate, sorbic acid, sodium propionate, and the like), or combinations thereof.

The excipient may comprise a lubricant. Examples of suitable lubricants include metal stearate such as magnesium stearate, calcium stearate, zinc stearate, a polyethylene glycol, a poloxamer, colloidal silicon dioxide, glyceryl behenate, light mineral oil, hydrogenated vegetable oils, magnesium lauryl sulfate, magnesium trisilicate, polyoxyethylene monostearate, sodium stearoyl fumarate, sodium stearyl fumarate, sodium benzoate, sodium lauryl sulfate, stearic acid, sterotex, talc, a polysorbate, palm monoglycerides, or combinations thereof.

The excipient may comprise a color additive. Suitable color additives include, but are not limited to, food, drug and cosmetic colors (FD&C), drug and cosmetic colors (D&C), or external drug and cosmetic colors (Ext. D&C). drug and cosmetic colors (D&C), or external drug and cosmetic colors (Ext. D&C). These colors or dyes, along with their corresponding lakes, and certain natural and derived colorants may be suitable for use in the compositions.

The excipient may comprise a flavoring agent. Flavoring agents may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oils (such as lemon oil, orange oil, grape and grapefruit oil), and fruit essences (such as apple, peach, pear, strawberry, raspberry, cherry, plum, pineapple, and apricot). In still another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; stevia-derived sweeteners; chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof. In still another embodiment, the excipient may include a taste-masking agent.

The excipient may comprise a taste-masking agent. Suitable taste masking agents include cellulose hydroxypropyl ethers (HPC); low-substituted hydroxypropyl ethers (L-HPC); cellulose hydroxypropyl methyl ethers (HPMC); methylcellulose polymers and mixtures thereof; polyvinyl alcohol (PVA); hydroxyethylcelluloses; carboxymethylcelluloses and salts thereof; polyvinyl alcohol and polyethylene glycol co-polymers; monoglycerides or triglycerides; polyethylene glycols; acrylic polymers; mixtures of acrylic polymers with cellulose ethers; cellulose acetate phthalate; or combinations thereof.

The excipient may comprise a stabilizer. Suitable stabilizers include alginates, agar, carrageen, cellulose and cellulose derivatives, guar gum, gelatin, gum Arabic, locust bean gum, pectin, starch, xanthan gum, and combinations thereof.

### C. Optional Sub-Coating

In some embodiments, the rumen-protected compositions may include an optional sub-coating disposed between the bioactive core and the coating comprising the chitosan organic salt. The sub-coating may be particularly beneficial when the physiologically active ingredient of the bioactive core includes lysine, or histidine or choline, which are highly water-soluble. Thus, the sub-coating may comprise a hydrophobic material to provide additional protection to the bioactive core.

The sub-coating when included may comprise an oil, including fully hydrogenated oils, partially hydrogenated oils, distilled oils, fractionated oils, lipids, fatty acids, fatty acid derivatives, epoxidized oils, and combinations thereof. The oil may be a plant-based oil selected from the group consisting of soybean oil, palm oil, rapeseed oil, cotton oil, linseed oil, castor oil, and other hydrophobic plant-based oils known in the art and combinations thereof. In an exemplary embodiment, the sub-coating includes soybean oil and palm oil.

The sub-coating when included may comprise a fatty acid or a derivative of a fatty acid. For example, the sub-coating may include propionic acid, butyric acid, valeric acid, caproic acid, enanthic acid, caprylic acid, capric acid, undecylic acid, lauric acid, myristic acid, palmitic acid, stearic acid, or other fatty acids and derivatives thereof, or any combination thereof.

The sub-coating when included may comprise a carbohydrate. In some examples, the sub-coating may include cellulose, methyl cellulose, carboxymethyl cellulose, hydroxypropyl cellulose, hydroxypropyl methylcellulose, alginates, pectin, or any combination thereof.

The sub-coating when included may comprise a protein derivative. For example, the sub-coating may include zein, gluten, soy, whey, casein, or any combination thereof.

The sub-coating may comprise a plasticizer. For example, the sub-coating may include monoglycerides such as acetylated monoglycerides, citrates such as triethyl citrate, acetylated triethyl citrate, trimethyl citrate, tributyl citrate, and trioctyl citrate, and combinations thereof.

When included, the optional sub-coating may be present in an amount from about 10% to about 30% by weight of the coated composition (i.e., the bioactive core+coating comprising chitosan organic salt+sub-coating). In some embodiments, the optional sub-coating may be present in an amount from about 10% to about 15%, about 10% to about 20%, about 10% to about 25%, about 10% to about 30%, about 15% to about 30%, about 20% to about 30%, about 25% to about 30%, about 15% to about 25%, about 15% to about 20%, or about 20% to about 25% by weight of the coated composition.

### D. Specific Exemplary Embodiments

In an exemplary embodiment, a rumen-protected composition of the present disclosure comprises a bioactive core comprising methionine in an amount from about 65% to about 75% by weight of the composition, and a coating comprising: a chitosan organic salt in an amount from about 2% to about 3% by weight of the composition; an emulsifier in an amount from about 0.5% to about 3% by weight of the composition; and a fatty acid source in an amount from about 10% to about 20% by weight of the composition.

In another exemplary embodiment, a rumen-protected composition of the present disclosure comprises a bioactive core comprising methionine in an amount from about 65% to about 75% by weight of the composition, and a coating comprising: chitosan acetate in an amount from about 2% to about 3% by weight of the composition; lecithin in an amount from about 0.5% to about 3% by weight of the composition; and a fatty acid source in an amount from about 10% to about 20% by weight of the composition.

In another exemplary embodiment, a rumen-protected composition of the present disclosure comprises a bioactive core comprising methionine in an amount from about 65% to about 75% by weight of the composition and lysine in an amount from about 1% to about 10% by weight of the composition, and a coating comprising chitosan organic salt in an amount from about 2% to about 3% by weight of the composition; an emulsifier in an amount from about 0.5% to about 3% by weight of the composition; and a fatty acid source in an amount from about 10% to about 20% by weight of the composition.

In another exemplary embodiment, a rumen-protected composition of the present disclosure comprises a bioactive core comprising lysine in an amount from about 70% to about 85% of the composition and a coating comprising chitosan organic salt in an amount from about 1% to about 3% by weight of the composition; an emulsifier in an amount from about 0.5% to about 3% by weight of the composition; and a fatty acid source in an amount from about 10% to about 20% by weight of the composition.

In another exemplary embodiment, a rumen-protected composition of the present disclosure comprises a bioactive core comprising histidine in an amount from about 55% to about 75% of the composition and a coating comprising chitosan organic salt in an amount from about 1% to about 3% by weight of the composition; an emulsifier in an amount from about 0.5% to about 3% by weight of the composition; and a fatty acid source in an amount from about 10% to about 20% by weight of the composition.

In another exemplary embodiment, a rumen-protected composition of the present disclosure comprises a bioactive core comprising choline in an amount from about 50% to about 75% of the composition and a coating comprising chitosan organic salt in an amount from about 1% to about 3% by weight of the composition; an emulsifier in an amount from about 0.5% to about 3% by weight of the composition; and a fatty acid source in an amount from about 10% to about 20% by weight of the composition.

### II. PELLET COMPOSITIONS

Further provided herein are rumen-protected pellet compositions. The pellet compositions comprise a physiologically active ingredient, including any physiologically active ingredient discussed in Section I(A)(i) above. In preferred embodiments, the pellet compositions do not comprise a coating as they have been shown to achieve acceptable levels of rumen protection and total bioavailability without the use of a coating. However, a coating may be added to provide additional rumen protection.

The physiologically active ingredient may comprise methionine. In general, the amount of methionine may range from about 30% to about 85% by weight of the pellet composition. In various embodiments, the amount of methionine may be at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the pellet composition. In another embodiment, the pellet composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % methionine by weight of the pellet composition.

The physiologically active ingredient may comprise lysine. In general, the amount of lysine may range from about 30% to about 85% by weight of the pellet composition. In various embodiments, the amount of lysine may be at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the pellet composition. In another embodiment, the pellet composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % lysine by weight of the pellet composition.

The physiologically active ingredient may comprise a combination of methionine and lysine. In these embodiments, the lysine may be present in an amount of about less than 10% by weight of the uncoated bioactive core; for example, less than 10 wt %, less than 9 wt %, less than 8 wt %, less than 7 wt %, less than 6 wt %, or less than 5 wt %. In another embodiment, the lysine may be present in an amount from about 1% to about 10% by weight of the uncoated bioactive core, for example, from about 1 wt % to about 2 wt %, about 1 wt % to about 3 wt %, about 1 wt % to about 4 wt %, about 1 wt % to about 5 wt %, about 1 wt % to about 6 wt %, about 1 wt % to about 7 wt %, about 1 wt % to about 8 wt %, about 1 wt % to about 9 wt %, about 2 wt % to about 10 wt %, about 3 wt % to about 10 wt %, about 4 wt % to about 10 wt %, about 5 wt % to about 10 wt %, about 6 wt % to about 10 wt %, about 7 wt % to about 10 wt %, about 8 wt % to about 10 wt %, or about 9 wt % to about 10 wt %.

The physiologically active ingredient may comprise histidine. In general, the amount of histidine may range from about 30% to about 85% by weight of the pellet composition. In various embodiments, the amount of histidine may be at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the pellet composition. In another embodiment, the pellet composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % histidine by weight of the pellet composition.

The physiologically active ingredient may comprise choline. In general, the amount of choline may range from about 30% to about 85% by weight of the pellet composition. In various embodiments, the amount of choline may be at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, or at least about 80 wt % of the pellet composition. In another embodiment, the pellet composition may comprise about 55 wt % to about 85 wt %, about 60 wt % to about 85 wt %, about 65 wt % to about 85 wt %, about 70 wt % to about 85 wt %, about 75 wt % to about 85 wt %, about 80 wt % to about 85 wt %, about 55 wt % to about 80 wt %, about 60 wt % to about 80 wt %, about 65 wt % to about 80 wt %, or about 65 wt % to about 75 wt % choline by weight of the pellet composition.

The pellet composition further comprises a chitosan organic salt. However, rather than coating the physiologically active ingredient with the chitosan organic salt, the chitosan organic salt is evenly distributed throughout the pellet. The chitosan organic salt may be any chitosan organic salt as described in Section I(B)(i) above or a combination thereof. In an exemplary embodiment, the chitosan organic salt is chitosan acetate.

The chitosan organic salt may be present in the pellet composition in an amount no less than about 0.05% by weight. The chitosan organic salt may be present in the coated composition in an amount of about 0.05% by weight to about 10% by weight. For example, the chitosan organic salt may be present in the pellet composition in an amount of about 0.05%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight. In still further embodiments, the chitosan organic salt may be present in the pellet composition in an amount of about 0.05% to about 1%, about 0.05% to about 2.5%, about 0.05% to about 5%, about 0.05% to about 7.5%, about 0.05% to about 10%, about 1% to about 10%, about 2.5% to about 10%, about 5% to about 10%, about 7.5% to about 10%, about 2.5% to about 7.5%, or about 2.5% to about 5% by weight.

The pellet composition may further comprise a fatty acid source dispersed throughout the pellet composition. The fatty acid source may be any fatty acid source described in Section I(A)(ii) above.

The pellet composition may further comprise an excipient dispersed throughout the pellet composition. The excipient may be any excipient described in Section I(A)(iii) above.

The pellet composition may further comprise a coating. The coating may further increase the rumen protection and the total bioavailability of the pellet composition. The coating may be any coating known in the art. Preferably, the coating is a coating described in Section I(B) above, or the coating may be the sub-coating described in Section I(D) above. In some preferred embodiments, the pellet composition does not include a coating.

### III. Feed Premixes

A further aspect of the present disclosure encompasses feed premixes comprising any of the compositions described above in Section I and/or Section II.

The coated rumen-protected composition as described in Section I may be present in the feed premix in an amount of about 99% or less, about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the feed premix.

The pellet composition as described in Section II may be present in the feed premix in an amount of about 99% or less, about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the feed premix

The feed premix may comprise one or more amino acids, amino acid analogs, vitamins, minerals, antioxidants, organic acids, polyunsaturated fatty acids, essential oils, enzymes, prebiotics, probiotics, herbal extracts, or pigments, examples of which are detailed above. The feed premix may also comprise one or more mycotoxin binders, approved antibiotics, antiparasitic agents, ionophores, or excipients.

Examples of suitable mycotoxin binders include, without limit, charcoal, activated carbon, silicates (e.g., phyllosilicates, tectosilicates, aluminosilicates, hydrated sodium calcium aluminosilicates, bentonites, zeolites, clinoptilolites, montmorillonites, and modified versions thereof), organic polymers (e.g., cellulose, glucomannans, peptidoglycans, and modified versions thereof), synthetic polymers (e.g., cholestyramine, polyvinylpyrrolidone, and the like), yeast cell wall extracts, and bacterial extracts. Non-limiting examples of antibiotics approved for use in livestock and poultry include bacitracin, carbadox, ceftiofur, enrofloxacin, florfenicol, laidlomycin, linomycin, oxytetracycline, roxarsone, tilmicosin, tylosin, and virginiamycin. Examples of suitable antiparasitic agents include but are not limited to abamectin, afoxolaner, albendazole, alphamethrin, amitraz, azamethiphos, carbaryl, chlorfenvinphos, chlorpyrifos, clorsulon, closantel, coumaphos, cyfluthrin, cyhalothrin, cymiazol, cypermethrin, cyromazine, ceclorcos, deltamethrin, diazinon, dichlorvos, dicyclanil, diflubenzuron, doramectin, dympilate, eprinomectin, ethion, febtantel, fenbendazole, fenitrothion, fenthion, fenvalerate, fipronil, fluazuron, flubendazole, flumethrin, imidacloprid, ivermectin, levamisole, lufenuron, malathion, mebendazole, metrifonate, methoprene, milbemycin oxime, monepantel, morantal, moxidectin, netobimin, niclosamide, nitroxinil, oxfendazole, oxibendazole, oxyclozanide, permethrin, phosmet, phoxim, piperazine, praziquantel, propoxur, pyrantel, rafoxanide, ribobendazole, rofenone, selamectin, spinosad, trichlorfon, thiabendazole, thiamethoxam, thiophanate, triclabendazole, and triflumuron. Suitable ionophores include but are not limited to bambermycin, decoquinate, diclazuril, lasalocid, maduramicin, monensin, narasin, nicarazin, nystatin, robenidine, salinomycin, semduramicin, variants, or derivatives thereof.

Suitable excipients include fillers, diluents, binders, pH modifiers, anti-caking agents, disintegrants, lubricants, dispersants, preservatives, flavoring agents, sweetening agents, taste masking agents, coloring agents, and combinations thereof. Examples of suitable fillers include, without limit, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol. Diluents suitable for inclusion include saccharides such as sucrose, dextrose, lactose, microcrystalline cellulose, fructose, xylitol, and sorbitol, polyhydric alcohols, starches, pre-manufactured direct compression diluents, and mixtures of any of the foregoing. Non-limiting examples of suitable binders include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons. Suitable pH modifiers include, without limit, sodium carbonate, sodium bicarbonate, citric acid, tartaric acid, and the like. Examples of anti-caking agents include magnesium stearate, magnesium sulfate, magnesium oxide, sodium bicarbonate, sodium silicate, silicon dioxide, talc, and combinations thereof. Suitable disintegrants include, without limit, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, tragacanth, and combinations thereof. Non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. Suitable dispersants include starch, alginic acid, polyvinylpyrrolidones, guar gum, kaolin, bentonite, purified wood cellulose, sodium starch glycolate, isamorphous silicate, and microcrystalline cellulose as high HLB emulsifier surfactants. Suitable examples of preservatives include, but are not limited to, antioxidants, such as a-tocopherol or ascorbate, and antimicrobials, such as parabens, chlorobutanol or phenol. Flavoring agents may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot. Suitable sweetening agents glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like. Taste-masking agents include cellulose hydroxypropyl ethers, low-substituted hydroxypropyl ethers, cellulose hydroxypropyl methyl ethers, alklylcelluloses, hydroxy- or carboxy-substituted alkyl celluloses, acrylic polymers, cellulose acetate phthalate, cyclodextrins, and mixtures thereof. Suitable color agents include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present composition depending on the embodiment.

The feed premix may be a pelleted mixture, a granular mixture, a particulate mixture, a tablet, or a capsule.

### IV. Feed Compositions

Another aspect of the present disclosure provides feed compositions comprising any of the compositions as described above in Section I and/or Section II and at least one nutritive agent.

The weight fraction of the coated rumen-protected composition described in Section I in the feed composition may be about 99% or less, about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The weight fraction of the pellet composition in the feed composition described in Section II may be about 99% or less, about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The at least one nutritive agent may be a carbohydrate source, a fat source, a protein source, an amino acid or derivative thereof, or combination thereof.

Suitable carbohydrate sources may be chosen from those known in the art and include, without limitation, alginate, arrowroot, barley, canola, cassava, corn, corn syrup, cottonseed meal, fructose, glucose, galactose, grain sorghum, kelp meal, lactose, maize, maltose, mannose, potatoes, oats, rice, rye, sago, sorbitol, soybeans, tapioca, wheat, wheat gluten, yam, and combinations thereof.

The fat source may be an inert fat or a non-inert fat. Non-limiting examples of non-inert fats include plant derived oils (e.g., canola oil, corn oil, cottonseed oil, palm oil, peanut oil, safflower oil, soybean oil, and sunflower oil), fish oils (e.g., menhaden oil, anchovy oil, albacore tuna oil, cod liver oil, herring oil, lake trout oil, mackerel oil, salmon oil, and sardine oil), animal fats (e.g., poultry fat, beef tallow, butter, pork lard, and whale blubber), yellow grease (i.e., waste grease from restaurants and low-grade fats from rendering plants), and combinations thereof. The non-inert fat source may also be a high fat product such as fish meal (e.g., menhaden meal, anchovy meal, herring meal, pollack meal, salmon meal, tuna meal, and whitefish meal), oilseeds (e.g., canola seeds, cottonseeds, flax seeds, linseeds, Niger seeds, sesame seeds, soy beans, and sunflower seeds), or distillers grains (e.g., dried distillers grains and solubles (DDGS) and wet distillers grains). The fat source may be a ruminally inert fat. Suitable examples of ruminally inert fats include calcium salts of palm fatty acids (e.g., MEGALAC®), saturated free fatty acids, or hydrogenated tallow (e.g., ALIFET®).

Suitable protein sources may be animal-derived proteins, plant-derived proteins, algal-derived proteins, or combinations thereof. In some embodiments, suitable sources of animal derived protein include blood meal, bone meal, fish meal, fish processing by-products, meat meal, meat and bone meal, poultry by-produce meal, feather meal, and combinations thereof. In other embodiments, suitable sources of plant-derived proteins include grains such as corn, oats, soybean, and the like; grain protein concentrates such as soy protein concentrate; legumes such as peas, lupine, alfalfa; distiller's grains; oilseed meals such as canola meal, cottonseed meal, flaxseed meal, soybean meal, sunflower seed meal; and combinations thereof. Suitable amino acids or derivatives thereof are described above.

The feed composition may also comprise at least one agent chosen from essential oils, metal chelates, minerals, amino acids, organic acids, vitamins, antioxidants, polyunsaturated fatty acids, prebiotics, probiotics, enzymes, ionophores, mycotoxin binders, antiparasitic agents, antibiotics, herbal extracts, pigments, excipients, or combinations thereof.

The feed composition may be formulated as pellets, granulates, extrudates, particulate blends, meal cakes, crumbled diets, gelled masses, and the like.

### V. Processes for Preparing Compositions

Further provided herein are methods of making the rumen-protected compositions of the present disclosure.

### A. Coated Rumen-Protected Compositions

The methods described herein may be used to make any composition described in Section I above. The process comprises providing a core and a coating composition and forming a coating over the core. The core may be a granular core, an extruded core, a crystalline core, a particulate core, and the like. Methods of forming granulated cores, extruded cores, crystalline cores, and particulate cores are generally known those having ordinary skill in the art.

In some embodiments, the cores may be formed in an extruder. The cores are formed by combining the physiologically active ingredient and optionally at least one excipient, and then extruding the combination. The extruder may be any extruder known in the art, such as a twin-screw extruder, a single screw extruder, a ram extruder, and the like.

In some embodiments, the core may be formed simply by providing the physiologically active ingredient in a granule, crystal, or particulate form. For example, crystals of histidine, such as histidine hydrochloride monohydrate crystals, may be combined optionally combined with at least one excipient to form the core of the composition.

The coating(s) may be formed by fluid bed coating, Wurster coating, spray coating, thermal spraying, cold spraying, spray gun coating, dip coating, vacuum film coating, and so forth. Such coating processes are well-known in the art. In a preferred embodiment, the coating layer is formed using a Wurster coating bed.

The weight of the coating relative to the weight of the coated composition (i.e., the bioactive core plus the coating) is not more than about 30% (i.e., 30 wt % coating). In some embodiments, the coating may be present in the coated composition in an amount from about 1% to about 30%, or from about 5% to about 25% on a dry weight basis. For instance, the weight percent of the coating may about 1%, about 2%, about 3%, about 4%, about 5%, about 6%, about 7%, about 8%, about 9%, about 10%, about 11%, about 12%, about 13%, about 14%, about 15% about 16%, about 17%, about 18%, about 19%, about 20%, about 21%, about 22%, about 23%, about 24%, or about 25%. In other embodiments, the weight percent of the coating may be about 10% to about 20%. For instance, the weight percent of the coating may be about 10%, about 11%, about 12%, about 13%, about 14%, about 15%, about 16%, about 17%, about 18%, about 19%, or about 20%. In preferred embodiments, the weight percent of the coating may be from about 15% to about 20%. For example, the weight percent of the coating may be about 15%, about 16%, about 17%, about 18%, about 19%, or about 20%.

The ingredients in the coating layer(s) are generally dissolved or dispersed in a solvent to form a coating solution prior to the coating step. The solvent may be a protic polar solvent, an aprotic polar solvent, or a nonpolar solvent. The solvent may comprise a mixture of two or more solvents, such as a mixture of acetic acid and water. In an exemplary embodiment, the solvent comprises a mixture of water and 1 wt % acetic acid. Preferably, the solvent is water. The duration of the coating process can and will vary depending upon the temperature, the concentration of the ingredients in the coating solution, and the desired coating level. The coating composition may be prepared at a temperature of about 50° C. and allowed to cool to room temperature.

The coating composition may be applied to the bioactive core using coating methods known in the art. The core may be uncoated or may already include one or more coating layers. In some embodiments, the coating composition is applied by Wurster coater. In such embodiments using a Wurster coating process, the inlet air of the Wurster coater may have a temperature of less than about 70° C.; for example, the inlet of the Wurster coater may have a temperature of less than about 70° C., less than about 65° C., less than about 60° C., or less than about 55° C. Preferably, the inlet of the Wurster coater is from about 50° C. to about 60° C. The product temperature (i.e., the temperature of the cores being coated) may be less than about 40° C.; for example, the product temperature may be less than about 40° C., less than about 35° C., or less than about 30° C. Preferably, the product temperature is from about 30° C. to about 35° C.

In additional embodiments, the coating composition may be applied by using a pan coater, such as a fully perforated pan coater. The core may be uncoated or may already include one or more coating layers. In such embodiments using a pan coating process, the inlet air of the pan coater may have a temperature of less than about 70° C.; for example, the inlet of the pan coater may have a temperature of less than about 70° C., less than about 65° C., or less than about 60° C. Preferably, the inlet of the pan coater is from about 60° C. to about 65° C. The product temperature (i.e., the temperature of the cores being coated) may be less than about 45° C.; for example, the product temperature may be less than about 45° C., less than about 40° C., or less than about 35° C. Preferably, the product temperature of the pan coater is from about 40° C. to about 35° C.

The coating composition may comprise any of the ingredients described above in Section IB. The coating composition may comprise at least about 1 wt % solids dissolved in the solvent such that the formulation has sufficient viscosity to be sprayable. For example, the coating composition may comprise at least about 1 wt % solids, at least about 5 wt % solids, at least about 10 wt % solids, at least about 15 wt % solids, at least about 20 wt % solids, at least about 25 wt % solids, at least about 30 wt % solids, at least about 35 wt % solids, at least about 40 wt % solids, at least about 45 wt % solids, or at least about 50 wt % solids.

The coating composition may comprise from about 1 wt % to about 10 wt % chitosan organic salt on a wet basis. The coating composition may comprise from about 1 wt % to about 2 wt %, about 1 wt % to about 3 wt %, about 1 wt % to about 4 wt %, about 1 wt % to about 5 wt %, about 1 wt % to about 6 wt %, about 1 wt % to about 7 wt %, about 1 wt % to about 8 wt %, about 1 wt % to about 9 wt %, about 1 wt % to about 10 wt %, about 2 wt % to about 10 wt %, about 3 wt % to about 10 wt %, about 4 wt % to about 10 wt %, about 5 wt % to about 10 wt %, about 6 wt % to about 10 wt %, about 7 wt % to about 10 wt %, about 8 wt % to about 10 wt %, or about 9 wt % to about 10 wt % of a chitosan organic salt on a wet basis.

The coating composition may comprise from about 1 wt % to about 10 wt % of an emulsifier on a wet basis. The coating composition may comprise from about 1 wt % to about 2 wt %, about 1 wt % to about 3 wt %, about 1 wt % to about 4 wt %, about 1 wt % to about 5 wt %, about 1 wt % to about 6 wt %, about 1 wt % to about 7 wt %, about 1 wt % to about 8 wt %, about 1 wt % to about 9 wt %, about 1 wt % to about 10 wt %, about 2 wt % to about 10 wt %, about 3 wt % to about 10 wt %, about 4 wt % to about 10 wt %, about 5 wt % to about 10 wt %, about 6 wt % to about 10 wt %, about 7 wt % to about 10 wt %, about 8 wt % to about 10 wt %, or about 9 wt % to about 10 wt % of an emulsifier on a wet basis.

The coating composition may comprise from at least about 15 wt % of a fatty acid source on a wet basis. For example, the coating composition may comprise at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, or at least about 25 wt % of a fatty acid source on a wet basis.

The coating composition may have a viscosity from about 1 cP to about 300 cP in order to be suitable for use as a sprayable coating. For example, the composition may have a viscosity from about 1 cP to about 50 cP, about 1 cP to about 100 cP, about 1 cP to about 150 cP, about 1 cP to about 200 cP, about 1 cP to about 250 cP, or about 1 cP to about 300 cP.

### B. Pellet Compositions

The methods described herein may be used to make the pellet compositions described in Section II above.

The method comprises mixing a powder comprising the physiologically active ingredient and the chitosan organic salt in distilled water, followed by vigorous mixing. The mixing may occur for a duration sufficient to ensure homogeneity of the mixture, which may be determined by visual confirmation. The viscosity of the mixture is preferably about 150 cP, but may vary depending on the ingredients used in the pellet composition. Additional ingredients as described in Section II may also be added to the distilled water and mixed.

The method further comprises drying the mixture. Preferably, the mixture is dried such that the dried composition is in the form of a pellet. The drying may be accomplished at a temperature from about 25° C. to about 50° C. The drying may occur overnight.

The method may further comprise coating the dried composition. The coating may be accomplished by any of the methods described in Section V(A) above.

### VI. Methods for Providing a Physiologically Active Ingredient to a Ruminant

Still another aspect of the present disclosure encompasses a method of providing a physiologically active ingredient to a ruminant. The physiologically active ingredient may be any physiologically active ingredient discussed in Section I or II above. The method comprises administering to the ruminant a rumen-protected composition of the present disclosure. Preferably, the composition is administered orally to the ruminant. The composition comprising may be any composition described above in Section I and/or Section II, a feed premix as described above in Section III, or a feed composition as described above in Section IV.

The rumen-protected composition may be administered to a variety of ruminants including, but not limited to, beef cattle, dairy cows, sheep, goats, bison, deer, moose, elk, reindeer, caribou, camels, giraffes, antelope, and llama.

The rumen-protected composition is stable in an aqueous solution under approximately neutral pH (i.e., about 6.0 to about 8.0). For example, the rumen-protected composition is stable at a pH level of about 6.0, about 6.5, about 7.0, and about 7.5. The rumen-protected composition releases the bioactive agent in an aqueous solution having a pH of about pH 3.0 or less; for example, about 3.0 or less, about 2.5 or less, about 2.0 or less, about 1.5 or less, or about 1.0 or less. Accordingly, the composition remains stable and is not degraded during the time in which the composition is in the rumen of the ruminant animal. Upon entry into abomasum, in which the pH is low, the composition releases the physiologically active ingredient. Accordingly, the compositions may be used for rumen bypass as the physiologically active ingredient is protected from degradation and/or hydrolysis by the coating and the physiologically active ingredient is selectively released in the low pH environment of the abomasum.

The compositions of the present disclosure have improved bioavailability as compared to other rumen-protected compositions. As used herein, bioavailability (also referred to as “total bioavailability”) is defined as the weight percent of a physiologically active ingredient that is absorbed by an animal through normal digestion as compared to the amount of the substance that was administered to the animal. As a non-limiting example, if 100 g of methionine is administered to an animal and 97 g of the methionine is absorbed by the animal through normal digestion, then the methionine is said to have a bioavailability of 97%.

The compositions of the present disclosure may have a bioavailability of at least 50%. The compositions of the present disclosure may have a bioavailability of at least about 50%, at least about 55%, at least about 60%, at least about 65%, at least about 70%, at least about 75%, at least about 80%, at least about 85%, or at least about 90%. The compositions of the present disclosure may have a bioavailability of about 70% to about 75%, about 75% to about 80%, about 80% to about 85%, about 85% to about 90%, about 90% to about 95%, or about 95% to about 99%. For example, the compositions of the present disclosure may have a bioavailability of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some aspects, the bioavailability of the composition may be determined by the bioavailability assay described in the examples herein, by the Boisen test (described in Boisen, S., et al. “Prediction of total tract digestibility of energy feedstuffs and pig diets by in vitro analyses, Anim. Feed Sci. Technol. 1997, 68, 277-286, the entire contents of which are incorporated by reference herein), by the Pulse-Dose method (described further in the Examples), and/or by the isotope methodology (described further in the Examples).

Furthermore, the rumen-protected compositions of the present disclosure have improved rumen bypass as compared to other rumen-protected compositions. “Rumen bypass” as used herein refers to the rate at which the amount of a bioactive agent does not degrade or dissolve in the rumen. As a non-limiting example, if 100 g of methionine is administered to an animal and 93 g of the methionine is not degraded or dissolved in the rumen before passing through to the lower digestive tract, then the methionine is said to have a rumen bypass rate of 93%.

The rumen bypass rate of the physiologically active ingredient in the rumen-protected compositions of the present disclosure may be at least about 45%, at least about 50%, at least about 55%, at least about 60%, at least about 65%, at least about 70%, at least about 75%, at least about 80%, at least about 85%, or at least about 90%. In some embodiments, the rumen bypass rate of the physiologically active ingredient in the rumen-protected compositions of the present disclosure may be about 70% to about 75%, about 75% to about 80%, about 80% to about 85%, about 85% to about 90%, about 90% to about 95%, or about 95% to about 99%. For example, the rumen bypass rate of the physiologically active ingredient in the rumen-protected compositions of the present disclosure of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some aspects, the rumen bypass rate of the physiologically active ingredient may be determined by the Buffer/Lipase Assay, which is described in the Examples herein.

Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

The term “weight percent of the coating,” which may be abbreviated as “wt % coating,” refers to the weight of the coating relative to the weight of the coated bioactive core. For instance, if the weight of the coated composition is 10 g, then a coating of 10 wt % means the weight of the coating is 1 g.

Unless specified otherwise, the amount of any component in a coating described herein is expressed as a percentage of the dry weight of the coating. The term “dry weight of the coating” refers to the sum of the weights of the various components in the coating composition, excluding any added water. For instance, if a coating composition consists of 3.0 g ethyl cellulose, 1.0 g plasticizer, 1.0 g chitosan organic salt and 95.0 g water, the chitosan salt is 20% of the dry weight of the coating [1.0/(3.0+1.0+1.0)].

### Exemplary Embodiments

Embodiment 1: A rumen-protected composition comprising a bioactive core comprising a physiologically active ingredient, the core surrounded by at least one coating layer, the at least one coating layer comprising:
(a) a chitosan organic salt;
(b) an emulsifier; and
(c) a fatty acid source,
wherein the coating is present in an amount of at least 10% by dry weight of the composition and the ratio of chitosan organic salt to emulsifier in the coating layer is less than about 2.5:1 by weight.

Embodiment 2: The rumen-protected composition of embodiment 1, wherein the physiologically active ingredient is selected from the group consisting of methionine, lysine, histidine, choline, and any combination thereof.

Embodiment 3: The rumen-protected composition of embodiment 1 or embodiment 2, wherein the physiologically active ingredient has a total bioavailability of at least about 70%.

Embodiment 4: The rumen-protected composition of any one of embodiments 1-3, wherein the physiologically active ingredient has a total bioavailability of at least about 80%.

Embodiment 5: The rumen-protected composition of any one of embodiments 1-4, wherein the physiologically active ingredient has a total bioavailability of at least about 90%.

Embodiment 6: The rumen-protected composition of any one of embodiments 1-5, wherein the physiologically active ingredient has a rumen bypass rate of at least about 70%.

Embodiment 7: The rumen-protected composition of any one of embodiments 1-6, wherein the physiologically active ingredient has a rumen bypass rate of at least about 80%.

Embodiment 8: The rumen-protected composition of any one of embodiments 1-7, wherein the chitosan organic salt is selected from the group consisting of chitosan acetate, chitosan glutamate, chitosan thiaminate, chitosan aspartate, chitosan nicotinate, and chitosan citrate.

Embodiment 9: The rumen-protected composition of any one of embodiments 1-8, wherein the chitosan organic salt is chitosan acetate.

Embodiment 10: The rumen-protected composition of any one of embodiments 1-9, wherein the emulsifier is selected from the group consisting of lecithin, monoglycerides, diglycerides, polyglycerol esters, propylene glycol esters, gums, waxes, phosphates, cellulose derivatives, and combinations thereof.

Embodiment 11: The rumen-protected composition of any one of embodiments 1-10, wherein the emulsifier is lecithin.

Embodiment 12: The rumen-protected composition of any one of embodiments 1-11, wherein the fatty acid source is selected from C₁₂ to C₂₂ saturated fats, C₁₂ to C₂₂ unsaturated fats, C₁₀ to C₂₂ hydrogenated triglycerides, and natural hydrogenated fats.

Embodiment 13: The rumen-protected composition of any one of embodiments 1-12, wherein the bioactive core comprises the physiologically active ingredient in an amount from about 50% to about 80% by weight of the rumen-protected composition, and the at least one coating layer comprises:
the chitosan organic salt in an amount from about 2% to about 3% by weight of the rumen-protected composition,
the emulsifier in an amount from about 0.5% to about 3% by weight of the rumen-protected composition, and
the fatty acid source in an amount from about 10% to about 20% by weight of the rumen-protected composition.

Embodiment 14: The rumen-protected composition of embodiment 13, wherein the physiologically active ingredient has a total bioavailability of at least 50% and the rumen bypass rate of the physiologically active ingredient is at least about 50%.

Embodiment 15: The rumen-protected composition of any one of embodiments 1-14, wherein the at least one coating layer does not include a fat.

Embodiment 16: The rumen-protected composition of any one of embodiments 1-15, wherein the ratio of the chitosan organic salt to the emulsifier in the coating layer is less than about 2:1 by weight.

Embodiment 17: The rumen-protected composition of any one of embodiments 1-16, wherein the ratio of the chitosan organic salt to the emulsifier in the coating layer is less than about 1:1 by weight.

Embodiment 18: The rumen-protected composition of any one of embodiments 1-17, further comprising a sub-coating disposed between the bioactive core and the at least one coating layer.

Embodiment 19: The rumen-protected composition of embodiment 18, wherein the sub-coating comprises an oil selected from the group consisting of soybean oil, palm oil, rapeseed oil, cotton oil, linseed oil, castor oil, and other hydrophobic plant-based oils known in the art and combinations thereof.

Embodiment 20: The rumen-protected composition of embodiment 19, wherein the sub-coating comprises soybean oil and palm oil.

Embodiment 21: The rumen-protected composition of embodiment 2, wherein the physiologically active ingredient is methionine.

Embodiment 22: The rumen-protected composition of embodiment 21, wherein the methionine is present in the rumen-protected composition in an amount from about 30% to about 85% by weight of the rumen-protected composition.

Embodiment 23: The rumen-protected composition of embodiment 2, wherein the physiologically active ingredient is a combination of methionine and lysine.

Embodiment 24: The rumen-protected composition of embodiment 23, wherein the methionine is present in the rumen-protected composition in an amount from about 30% to about 85% by weight of the rumen-protected composition.

Embodiment 25: The rumen-protected composition of embodiment 23, wherein the lysine is present in the rumen-protected composition in an amount from about 1% to about 10% by weight of the rumen-protected composition.

Embodiment 26: The rumen-protected composition of embodiment 2, wherein the physiologically active ingredient is lysine.

Embodiment 27: The rumen-protected composition of embodiment 26, wherein the lysine is present in the rumen-protected composition in an amount from about 30% to about 85% by weight of the rumen-protected composition.

Embodiment 28: The rumen-protected composition of embodiment 2, wherein the physiologically active ingredient is histidine.

Embodiment 29: The rumen-protected composition of embodiment 28, wherein the histidine is present in the rumen-protected composition in an amount from about 30% to about 85% by weight of the rumen-protected composition.

Embodiment 30: The rumen-protected composition of any one of embodiments 1-29, wherein the bioactive core further comprises a fatty acid source.

Embodiment 31: The rumen-protected composition of any one of embodiments 1-30, wherein the composition further comprises an excipient.

Embodiment 32: The rumen-protected composition of embodiment 2, wherein the physiologically active ingredient is choline.

Embodiment 33: The rumen-protected composition of embodiment 32, wherein the choline is present in the rumen-protected composition in an amount from about 30% to about 85% by weight of the rumen-protected composition.

Embodiment 34: The rumen-protected composition of embodiment 2, wherein the physiologically active ingredient comprises choline and methionine.

Embodiment 35: The rumen-protected composition of embodiment 34, wherein the choline is present in the bioactive core of the rumen-protected composition in an amount from about 60% to about 70% by weight of the bioactive core, and the methionine is present in the bioactive core of the rumen-protected composition in an amount from about 15% to about 20% by weight of the bioactive core.

Embodiment 36: The rumen-protected composition of embodiment 2, wherein the physiologically active ingredient comprises choline, histidine, and methionine.

Embodiment 37: The rumen-protected composition of embodiment 36, wherein the choline is present in the bioactive core of the rumen-protected composition in an amount from about 40% to about 60% by weight of the bioactive core, the histidine is present in the bioactive core of the rumen-protected composition in an amount from about 10% to about 30% by weight of the bioactive core, and the methionine is present in the bioactive core of the rumen-protected composition in an amount from about 5% to about 10% by weight of the bioactive core.

Embodiment 38: The rumen-protected composition of embodiment 2, wherein the physiologically active ingredient comprises choline, lysine, and methionine.

Embodiment 39: The rumen-protected composition of embodiment 38, wherein the choline is present in the bioactive core of the rumen-protected composition in an amount from about 40% to about 60% by weight of the bioactive core, the lysine is present in the bioactive core of the rumen-protected composition in an amount from about 5% to about 10% by weight of the bioactive core, and the methionine is present in the bioactive core of the rumen-protected composition in an amount from about 10% to about 30% by weight of the bioactive core.

Embodiment 40: A rumen-protected composition comprising a bioactive core comprising a physiologically active ingredient selected from the group consisting of methionine, lysine, histidine, and any combination thereof; the core surrounded by at least one coating layer, the coating layer comprising:
(a) chitosan acetate;
(b) lecithin; and
(c) a fatty acid source, wherein the coating is present in an amount of at least 10% by dry weight of the composition and the ratio of chitosan acetate to lecithin in the coating layer is less than about 2.5:1.

Embodiment 41: A rumen-protected pellet composition comprising a bioactive core, the bioactive core comprising a physiologically active ingredient and a chitosan organic salt.

Embodiment 42: The rumen-protected pellet composition of embodiment 41, wherein the physiologically active ingredient is selected from the group consisting of methionine, lysine, histidine, choline, and any combination thereof.

Embodiment 43: The rumen-protected pellet composition of embodiment 41 or embodiment 42, wherein the composition does not include a coating.

Embodiment 44: The rumen-protected pellet composition of any one of embodiments 41-43, wherein the chitosan organic salt is selected from the group consisting of chitosan acetate, chitosan glutamate, chitosan thiaminate, chitosan aspartate, chitosan nicotinate, and chitosan citrate.

Embodiment 45: The rumen-protected pellet composition of any one of embodiments 41-44, wherein the chitosan organic salt is chitosan acetate.

Embodiment 46: The rumen-protected pellet composition of any one of embodiments 41-45, further comprising an emulsifier.

Embodiment 47: The rumen-protected pellet composition of embodiment 46, wherein the emulsifier is selected from the group consisting of lecithin, monoglycerides, diglycerides, polyglycerol esters, propylene glycol esters, gums, waxes, phosphates, cellulose derivatives, and combinations thereof.

Embodiment 48: The rumen-protected pellet composition of embodiment 46, wherein the emulsifier is lecithin.

Embodiment 49: The rumen-protected pellet composition of any one of embodiments 41-48, further comprising a fatty acid source.

Embodiment 50: The rumen-protected pellet composition of embodiment 49, wherein the fatty acid source is selected from C₁₂ to C₂₂ saturated fats, C₁₂ to C₂₂ unsaturated fats, C₁₀ to C₂₂ hydrogenated triglycerides, and natural hydrogenated fats.

Embodiment 51: The rumen-protected pellet composition of any one of embodiments 41-50, wherein the physiologically active ingredient is methionine.

Embodiment 52: The rumen-protected pellet composition of embodiment 51, wherein the methionine is present in the rumen-protected pellet composition in an amount from about 30% to about 85% by weight of the rumen-protected pellet composition.

Embodiment 53: The rumen-protected pellet composition of any one of embodiments 41-52, wherein the physiologically active ingredient is a combination of methionine and lysine.

Embodiment 54: The rumen-protected pellet composition of embodiment 53, wherein the methionine is present in the rumen-protected pellet composition in an amount from about 30% to about 85% by weight of the rumen-protected pellet composition.

Embodiment 55: The rumen-protected pellet composition of embodiment 53, wherein the lysine is present in the rumen-protected pellet composition in an amount from about 1% to about 10% by weight of the rumen-protected pellet composition.

Embodiment 56: The rumen-protected pellet composition of any one of embodiments 41-55, wherein the physiologically active ingredient is lysine.

Embodiment 57: The rumen-protected pellet composition of any one of embodiments 41-56, wherein the lysine is present in the rumen-protected pellet composition in an amount from about 30% to about 85% by weight of the rumen-protected pellet composition.

Embodiment 58: The rumen-protected pellet composition of any one of embodiments 41-57, wherein the physiologically active ingredient is histidine.

Embodiment 59: The rumen-protected pellet composition of embodiment 58, wherein the histidine is present in the rumen-protected composition in an amount from about 30% to about 85% by weight of the rumen-protected composition.

Embodiment 60: The rumen-protected pellet composition of any one of embodiments 41-59, wherein the physiologically active ingredient is choline.

Embodiment 61: The rumen-protected pellet composition of embodiment 60, wherein the choline is present in the rumen-protected pellet composition in an amount from about 30% to about 85% by weight of the rumen-protected pellet composition.

Embodiment 62: The rumen-protected pellet composition of any one of embodiments 41-61, wherein the physiologically active ingredient comprises choline and methionine.

Embodiment 63: The rumen-protected pellet composition of any one of embodiments 41-62, wherein the physiologically active ingredient comprises choline, histidine, and methionine.

Embodiment 64: The rumen-protected pellet composition of any one of embodiments 41-63, wherein the physiologically active ingredient comprises choline, lysine, and methionine.

Embodiment 65: The rumen-protected composition of any one of embodiments 1-40 or the rumen-protected pellet composition of any one of embodiments 41-64, wherein the composition does not include chitosan.

Embodiment 66: The rumen-protected composition of any one of embodiments 1-40 or the rumen-protected pellet composition of any one of embodiments 41-64, wherein the composition does not include an inorganic salt of chitosan.

Embodiment 67: A method of making a rumen-protected composition, the method comprising coating a bioactive core comprising a physiologically active ingredient with a coating composition, wherein:
the physiologically active ingredient is selected from the group consisting of methionine, lysine, histidine, choline, and any combination thereof; and the coating composition comprises a chitosan organic salt, an emulsifier, and a fatty acid source.

### Examples

### Example 1: pH Response of Polymer Coatings

Rumen protective coating need to be stable at neutral pH but dissolve at low pH. As such, several synthetic and chitosan-based polysaccharide polymers were formulated as films and tested for pH response. Dried films were subjected to pH 2.0 and pH 7.0. A film sample was determined to have a positive pH response when the film dissolved in the acidic solution but remained intact in the neutral solution. A sample was determined to be pH unresponsive when it dissolves or softens in both the acidic and neutral solutions. Table 1 provides a compilation of the results from these tests.

TABLE 1
pH Responsiveness of Films

| | | | | |
|---|---|---|---|---|
| | | pH 2.00 | pH 7.00 | |
| Sample | Film Appearance | buffer | buffer | pH response |

Chitosan polysaccharides

| | | | | |
|---|---|---|---|---|
| Chitosan | powder | inert | inert | Not responsive |
| Chitosan chloride | dry brittle material | Dissolves | Dissolves | Not responsive |
| Chitosan acetate | brown, wrinkled intact | Dissolves | inert | Responsive |
| | film | | | |
| Chitosan glutamate | dry brittle intact film | Dissolves | inert | Responsive |
| Chitosan lactate | dry soft film | Dissolves | soft gelatin- | Not responsive |
| | | | like | |
| Chitosan thiaminate | dry particulate brittle | Dissolves | inert | Responsive |
| | film | | | |
| Chitosan aspartate | dry brittle film | Dissolves | inert | Responsive |
| Chitosan nicotinate | dry brittle film | Dissolves | inert | Responsive |
| Chitosan citrate | dry flat film | Dissolves | inert | Responsive |
| Chitosan acetate | dry wrinkled film | Dissolves | inert | Responsive |

Synthetic Polymers

| | | | | |
|---|---|---|---|---|
| Basic butylated | clear flat film | Dissolves | smoky white | Responsive |
| methacrylate | | | film | |
| copolymer | | | | |
| Poly 2-vinylpyridine | white powder in clear | Dissolves, | inert | Responsive |
| co styrene | solutions | foams | | |

Chitosan alone was found to be inert, insoluble at ambient temp, and showed no pH trigger functionality. However, other chitosan salts tried (at film response level) showed functionality including chitosan glutamate, chitosan aspartate, chitosan thiaminate, chitosan nicotinate, and chitosan citrate, as shown in Table 1. Chitosan chloride and chitosan lactate were also found to be non-responsive in pH film tests. Chitosan Acetate was selected for further experimentation though other forms may be used.

### Example 2: Characterization of Chitosan Acetate

Chitosan acetate raw material from different suppliers was evaluated using gel permeation chromatography (GPC), also known as size exclusion chromatography (SEC), using Pullulan narrow standards from Shodex (Product Code—F8400000; Product Name—Standard P-82) consisting of eight separate standards with approximate peak molecular weight (Mp)-f—6,300, 9,800, 22,000, 49,700, 107,000, 216,000, 334,000, and 739,000 Da.

Chitosan acetate solutions (1 wt %) were prepared in warm water (<50° C.) in less than 2 hours and kept at ambient temperature. The mixtures were free flowing, making them easy to handle during the process. Dry powder chitosan acetate was also characterized for comparison. The characteristics of the tested solutions are shown in Table 1.

TABLE 2
GPC Evaluation - Molecular Weight Distribution

| SUPPLIER | FORM | Mw | Mn | Mp | Mz | Mw/Mn |
|---|---|---|---|---|---|---|
| Supplier #1 | Dry powder | 381,800 | 67,700 | 250,200 | 1,046,000 | 5.64 |
| Supplier #1 | 1 wt % Solution | 281,200 | 50,400 | 232,700 | 743,000 | 5.58 |
| Supplier #2 | Liquid | 601,600 | 144,900 | 193,400 | 2,900,000 | 4.15 |

Mw = Weight Average Molecular Weight
Mn = Number Average Molecular Weight
Mp = Peak Molecular Weight
Mz = Z Average Molecular Weight
Mw/Mn = Polydispersity

Next, chitosan acetate solutions prepared in warm and hot water were compared. A chitosan acetate solution (1 wt %) was prepared in warm water (<50° C.) in less than 2 hours and then kept at ambient temperature. A second solution was made that was instead dissolved in hot water (˜70° C.), with continuous mixing. The solutions were analyzed using a GPC. There was no significant molecular weight shift in chitosan acetate prepared at room temperature (see Table 3). However, there was a notable shift in both Mw/Mn/Mp weights to lower values when the chitosan acetate was prepared in hot water.

TABLE 3
Characteristics of Chitosan Acetate Solutions

| SUPPLIER | FORM | Mw | Mn | Mp | Mz | Mw/Mn |
|---|---|---|---|---|---|---|
| Supplier #1 | Dry powder | 400,631 | 71,962 | 243,386 | 1,629,163 | 5.57 |
| Supplier #1 | Warm water | 416,369 | 81,120 | 230,258 | 1,664,721 | 5.13 |
| Supplier #1 | Hot water | 195,307 | 51,063 | 103,308 | 1,660,005 | 3.82 |

### Example 3: Compatibility of Chitosan with Other Ingredients

Compatibility of chitosan acetate with other ingredients such as fatty acids was tested. Chitosan acetate solutions were mixed at high shear with other ingredients such as fatty acids including stearic acid, palmitic; fatty acid salts including sodium stearate, calcium stearate; emulsifiers including lecithin, Tween 80, Gum acacia, waxes, epoxidized soybean oil, glycosperse, potassium caseinate, and sodium dodecyl sulfate to determine whether a free flowing one phase mixture was formed. The mixture must be sprayable in order to be used in a coating bed system.

A dryfilm was prepared by air-drying and tested for pH responsiveness in pH 2.00 and pH 7.00 buffers as described before. The results are shown in Table 4. As seen in Table 4, Chitosan acetate formed a single phase dispersion with stearic acid (1 wt %) and lecithin (0.5 wt %), stearic acid (1 wt %) and Gum Acacia (0.5 wt %), stearic acid (1 wt %) and sodium stearate (0.5 wt %), and stearic acid (1 wt %) and Tween 80 (0.5 wt %). Combinations of chitosan acetate with stearic acid (1 wt %) and with steaeric acid (1 wt %) and sodium dodecyl sulfate (0.5 wt %) were not sprayable.

TABLE 4
Compatibility Studies of Chitosan Acetate with Other Ingredients

| Coating dispersion in | | pH 2.00 | pH 7.00 | |
|---|---|---|---|---|
| distilled water | Dispersion | buffer | buffer | pH Response |
| Chitosan acetate (1 wt %) + | turbid, shows phase | Dissolves | inert | Positive, NOT |
| stearic acid (1 wt %) | separation | | | sprayable |
| Chitosan acetate (1 wt %) + | smooth dispersion, | Dissolves | inert | Positive, |
| stearic acid (1 wt %) + | single phase | | | sprayable |
| lecithin (0.5 wt %) | | | | |
| Chitosan acetate (1 wt %) + | smooth dispersion, | Dissolves | inert | Positive, |
| stearic acid (1 wt %) + Gum | single phase | | | sprayable |
| Acacia (0.5 wt %) | | | | |
| Chitosan acetate (1 wt %) + | shows phase | NA | NA | Negative, NOT |
| stearic acid (1 wt %) + | separation, and | | | sprayable |
| sodium dodecyl sulfate (0.5 | precipitates | | | |
| wt %) | | | | |
| Chitosan acetate (1 wt %) + | smooth dispersion, | Dissolves | inert | Positive |
| stearic acid (1 wt %) + | single phase | | | |
| sodium stearate (0.5 wt %) | | | | |
| Chitosan acetate (1 wt %) + | smooth dispersion, | Dissolves | inert | Positive |
| stearic acid (1 wt %) + Tween | single phase | | | |
| 80 (0.5 wt %) | | | | |

### Example 4: Chitosan Acetate Film Response to pH

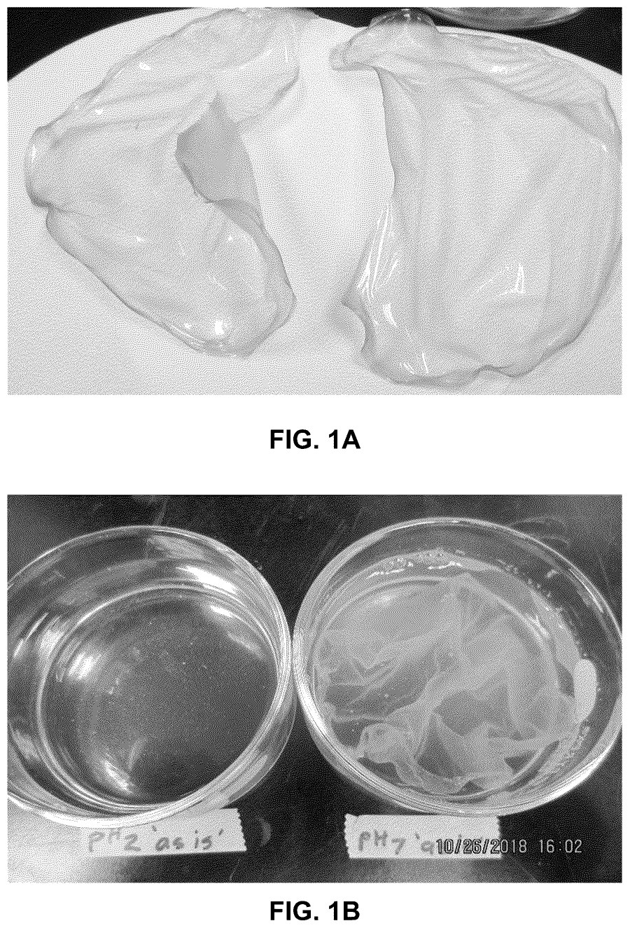

The pH responsiveness of the chitosan acetate samples was then tested by forming a dried film from the chitosan acetate solution at 3 wt % and 3.9 wt % and incubating the films in jars of 2.00 pH and 7.00 pH buffer solutions at 39° C. for 2 hours. A sample was determined to have a positive pH response when the film dissolved in the acidic solution but remained intact in the neutral solution. A sample was determined to be pH unresponsive when it dissolves or softens in both the acidic and neutral solutions. Residual acetic acid content of the chitosan acetate in solution was also determined. The results are shown in Table 7. It was determined that the chitosan acetate remained pH responsive at a maximum acetic acid concentration of 2% by weight of chitosan acetate. The chitosan acetate films before and after incubation are shown in FIGS. 1A and 1B, respectively.

TABLE 5
Acetic Acid pH Responsiveness

| | | | Specified | Analyzed | |
|---|---|---|---|---|---|
| | | | Acetic | Acetic | |
| | | | Acid | Acid | |
| Sample | Form | Source | (% wt/wt) | (% wt/wt) | Performance |
| Chitosan | Powder | Supplier | <20 | 1.1 | pH |
| Acetate | | #1 | | | Responsive |
| Chitosan | Liquid | Supplier | 6.0 | 6.0 | Not pH |
| Acetate, 3.0 | | #2 | | | responsive |
| wt % | | (batch 1) | | | |
| Chitosan | Liquid | Supplier | 2.0 | 2.0 | pH |
| Acetate, 3.9 | | #2 | | | responsive |
| wt % | | (batch 2) | | | |

### Example 5: Comparing Chitosan and Chitosan Acetate Response to pH

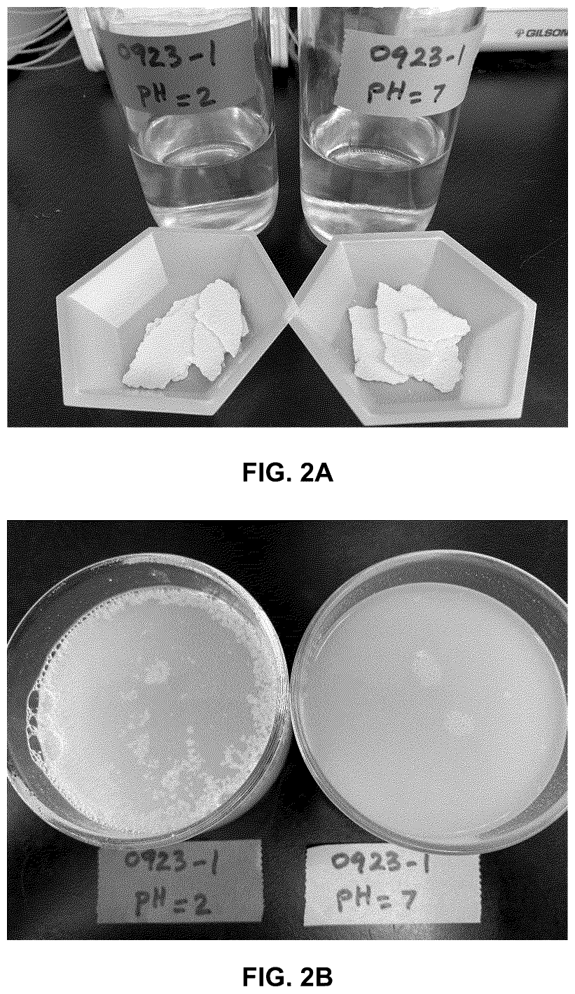

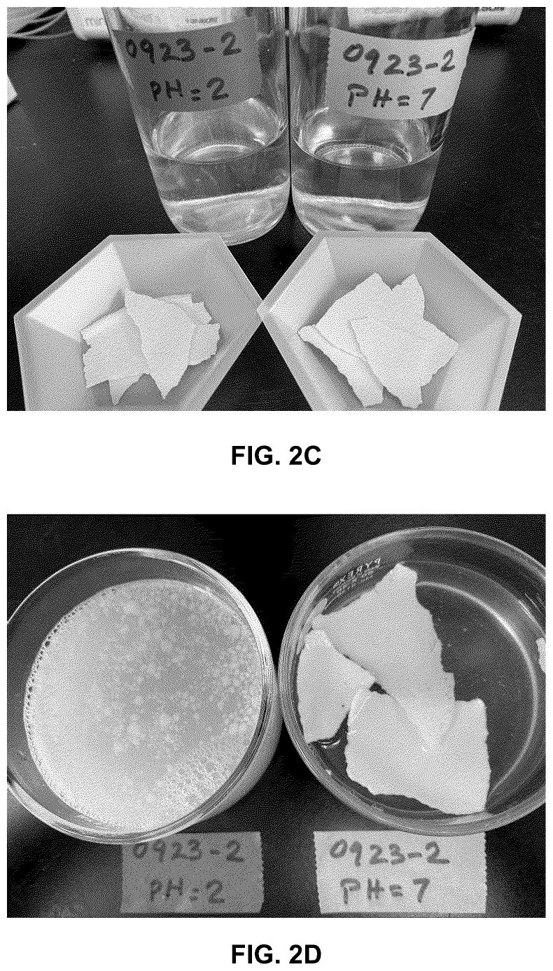

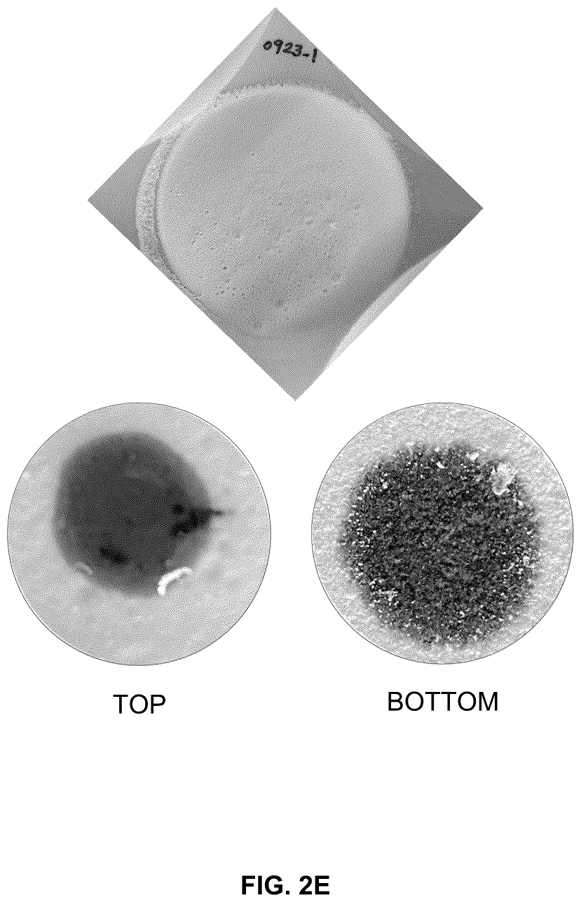

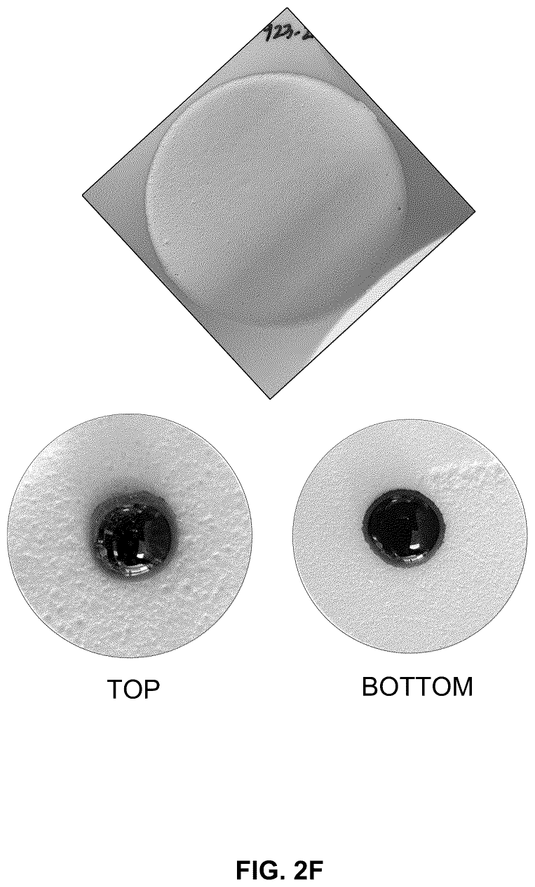

For further comparison, an attempt was made to produce coating compositions that included chitosan and chitosan acetate. However, a sprayable film-forming composition could not be achieved with chitosan. Therefore, hard shell coating compositions of chitosan and chitosan acetate coating compositions were prepared. The chitosan coating composition is shown in FIG. 2A and the chitosan acetate coating composition is shown in FIG. 2C prior to exposure to a buffer solution of 2.00 and a buffer solution of 7.00. Both coating compositions were then placed in the buffer solutions at 39° C. for about 2 hours.

FIGS. 2B and 2D show the chitosan coating composition and the chitosan acetate coating composition after exposure to the buffer solutions, respectively. The coating compositions contained about 8 wt % lecithin, about 84 wt % of a blend of stearic acid and palmitic acid, and the balance of either chitosan or chitosan acetate. As can be seen, the coating composition comprising chitosan dissolved in both the pH 2 buffer and the pH 7 buffer, further showing that a chitosan coating lacks a pH response and is unsuitable for use in a composition of the present disclosure. The composition comprising chitosan acetate, in contrast, did not dissolve in the pH 7 buffer, but did dissolve in the pH 2 buffer. The chitosan acetate coating composition therefore was pH-responsive and suitable for use in a rumen-protected composition.

The coating compositions were also dyed with drops of iodine. FIG. 2E shows the chitosan coating composition about 2 minutes after placing the iodine and FIG. 2F shows the chitosan acetate coating composition about 15 minutes after placing the iodine. It can be seen from FIG. 2E that the chitosan was mainly located on the bottom surface of the coating composition, since the dyed portion mainly appeared on the bottom of the coating. The porosity of the chitosan composition allowed the iodine to penetrate the film with ease compared to the chitosan acetate coating composition, which did not show any spread of the iodine drop over a relatively longer time period.

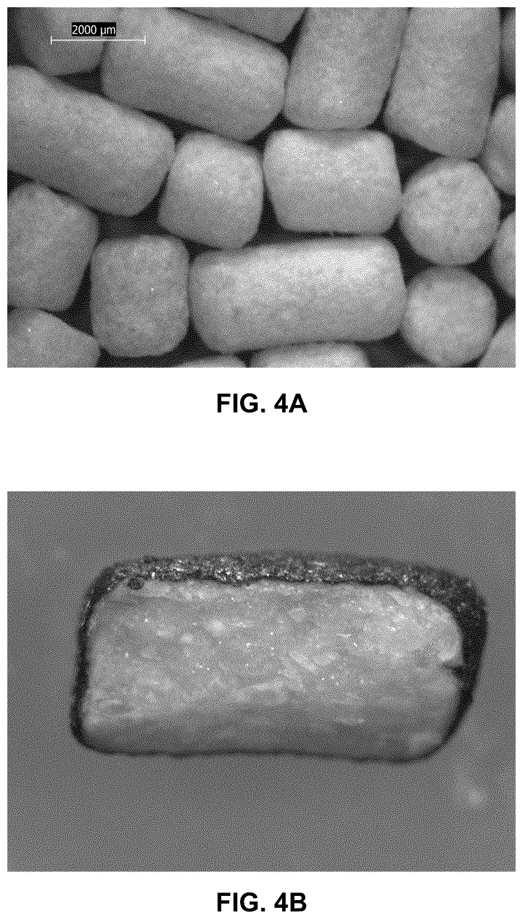

Further testing of the coating compositions for pH triggers was conducted. First, chitosan powder was added to a pH 2.0 buffer and a pH 7.0 buffer for 20 hours at 39° C. The solutions were then dried. FIG. 4A shows the chitosan powder in the buffer (top) and after drying (bottom). The chitosan did not dissolve in either of the buffer solutions, and a dry chitosan powder was recovered.

Next, a mixture of chitosan and lecithin was added to a pH 2.0 buffer and a pH 7.0 buffer for 20 hours at 39° C. The solutions were then dried. FIG. 4B shows the mixture dissolved in the buffers (top) and after drying (bottom). In the pH 2.0 buffer, the chitosan and the lecithin formed a solid mass that separated from the buffer solution. After drying, the solid mass formed a gummy material with particles of chitosan on the surface. In the pH 7.0 buffer, the lecithin emulsified in the solution but the chitosan remained as a solid.

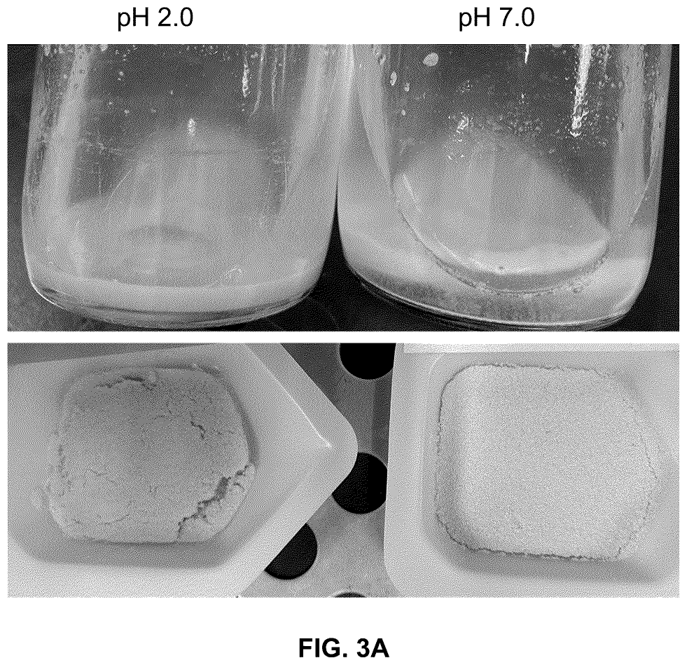

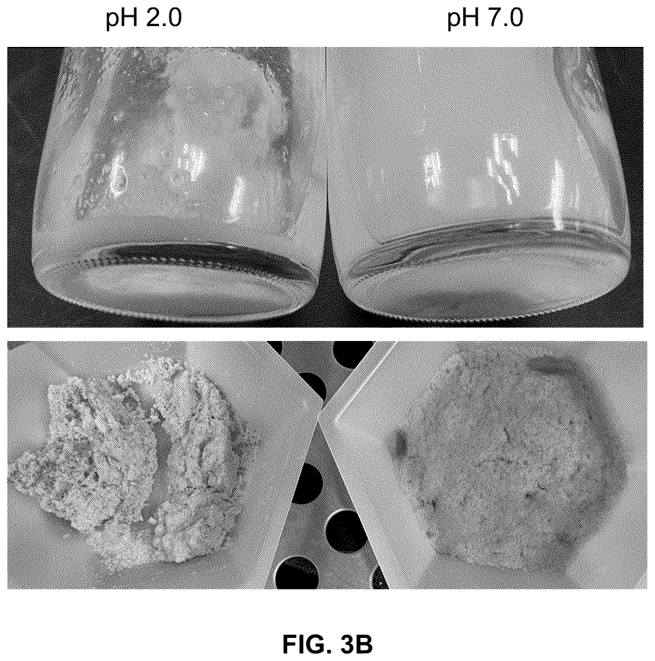

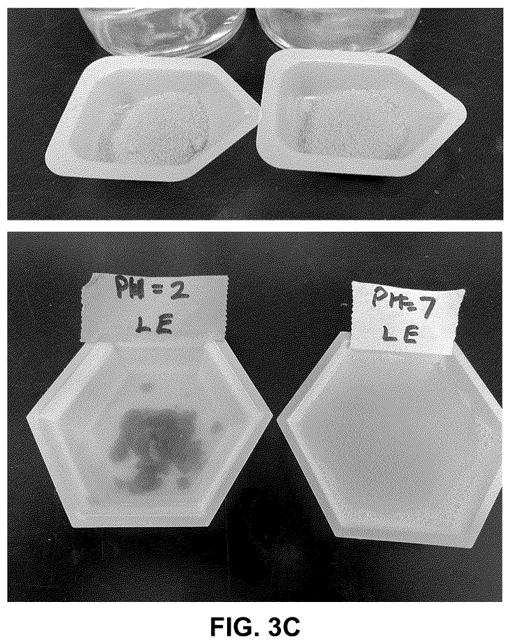

Finally, lecithin alone was added to a pH 2.0 buffer and a pH 7.0 buffer for 20 hours at 39° C. The solutions were then dried. FIG. 3C shows the dry lecithin before being added to the buffer solutions (top) and the lecithin in each of the buffer solutions (bottom). It is clear that the lecithin alone coagulates in the pH 2.0 buffer, but emulsifies to a turbid solution in the pH 7.0 buffer.

### Example 6: Preparation of a Coating Composition

A suitable coating composition consisting of a pH responsive chitosan salt, stearic acid, lecithin and water was prepared as shown in Table 5. The coating composition was prepared by dissolving 33 g of chitosan acetate from Supplier #1 in 967 g of warm distilled water (˜50° C.) to form 3.3% (wt %) solution. About 528 g of chitosan acetate solution was added to a mixture of 17 g of deoiled lecithin and 271 g of distilled water in a 2-liter vessel, and briefly homogenized to form a uniform dispersion. Approximately 183 g of stearic acid powder was gently added to the mixture followed by gentle overhead mixing to achieve wetting but still with visible large, clumped particles. The resultant mixture was homogenized with a high shear homogenizer at approximately 7500 rpm (Silverson 5L, rot-r—0“32” (8.00 mm), stat-r—1“38” (35.00 mm) for about 5 minutes. The rumen-protected composition was smooth, free flowing, and sprayable in a fluid bed system at approximately 40° C.

TABLE 6
Aqueous Dispersion Composition

| | | wt % of total | Weight of | wt % of |
|---|---|---|---|---|
| | Weight (g) | formula | Solids (g) | solids |
| Water | 78 | 78.0% | | |
| Stearic acid | 18.4 | 18.4% | 18.4 | 83.6% |
| Chitosan acetate | 1.8 | 1.8% | 1.8 | 8.2% |
| Lecithin, deoiled | 1.8 | 1.8% | 1.8 | 8.2% |
| Total | 100 | 100.0% | 22.0 | 100.0% |

The viscosity of the aqueous dispersion was determined using the Brookfield Viscometer with LVDVII*Digital, and spindle CPE-41 at 25.0° C. and the particle size was determined using the Horiba LA950 at 25.0° C., the results of which are shown in Table 6. Aqueous dispersion 1 was made as per formulation in Table 5; in dispersion 2, lecithin was replaced by Tween 80 (polysorbate 80) as the emulsifier.

TABLE 7
Viscosity and particle size (PSD) of aqueous
dispersion coating used in the application

| Formulation | Viscosity (cP) | PSD (μm) |
|---|---|---|
| Aqueous dispersion 1 | 44.0 | 45.2 |
| Aqueous dispersion 2 | 44.5 | 48.8 |
| Average | 44.3 | 47.0 |

The dispersion was sprayable at ambient temperature without any significant settling for more than 2 hours.

### Example 7: Preparation and Coating of Triggered Released Capsule Formulations

Triggered release rumen-protected methionine (Capsule #1), is a triggered release methionine formulation coated with a coating composition containing a chitosan organic salt. The formulation was composed of greater than 70 wt % of DL methionine, 0-15 wt % fatty acids blend, 0-8 wt % hydrogenated vegetable oil, less than 2 wt % lecithin, and less than 2 wt % chitosan acetate, as the chitosan organic salt.

Trigger release capsule compositions were prepared as provided here. Approximately 120 g of chitosan acetate powder was added to 3,380 g distilled water at room temperature or warm water (approximately 50° C.) and mixed using an overhead mixer for about 1 hour to ensure complete dissolution. The solution (about 3,500 g) was 3.32 wt % and was ready for use in preparing the coating dispersion. In general, this solution may be prepared a few days in advance and kept at room temperature before use.

Alternatively, a chitosan acetate solution was prepared from chitosan flakes. For this, approximately 66 g of chitosan flakes was added to 1934 g of 1% acetic acid solution in a 4 L vessel. An overhead mixer was attached to gently mix the solution to complete dissolution (about 2 hours). The solution (about 2,000 g) contained about 3.3 wt % chitosan acetate and was ready for use after it was left to de-bubble and settle overnight.

To prepare a coating dispersion for 1 batch (1,200 g), about 634 g of chitosan acetate solution was added to a 4 L vessel followed by 21 g of lecithin (de-oiled), and 27 g of distilled water. Using a homogenizer equipped with a high shear generator dispersing unit, Silverson (L5M-A), the mix was homogenized briefly (about 1 min at 3000 rpm) to obtain a smooth dispersion. To the mixture, about 220 g of fatty acids blend (Tristar 149) of stearic acid and palmitic acid powder was added slowly ensuring all large junks of materials are broken to small particles. Finally, a tan colored smooth dispersion was formed (approximately 22% solids) using a homogenizer at high shear for effective dispersion (approximately 7000 rpm for 5 min) for application at room temperature conditions.

Methionine cores were coated using a fluid bed system. For this, about 934 g of cylindrical methionine cores (approximately 2 mm in diameter) were charged to a 2 L fluid bed processor equipped with a Wurster insert and conditioned to 45-50° C. for coating. The coating dispersion, kept at room temperature, was sprayed to the cores using a peristaltic pump at a fluidization air flow rate of 27 SCFM, an inlet air temperature of 45° C., a product temperature of 32° C., an outlet temperature of 27° C., a spray rate of 6.0 g/min, an atomization air pressure of 12 psi, and Magna flow pressure of 50 psi for approximately 164 min. About 1090 g of triggered release capsules were produced, showing approximately 16.7% coating weight, i.e. (1090-934)/934=16.7% weight gain. The activity of methionine produced by this method was approximately 71% methionine, i.e. (core is 85% methionine×[100%×16.7%]coating)=(85%×83.3%)=70.8% methionine. Exemplary capsules are shown in FIGS. 3A and 3B.

### Example 8: Effect of Acetic Acid on Capsule Formulations

Next, the effect of excess acetic acid on encapsulated formulations (capsules) prepared from different chitosan acetate samples was tested. Capsule formulations produced with different sources of chitosan acetate are shown in Table 8. The fatty acid comprised a blend of stearic acid and palmitic acid. The lipids comprised a mixture of fatty acids and hydrogenated vegetable oils. Chitosan acetate in R4 was supplied as liquid (3 wt % chitosan acetate), which had a high acetic acid content (6 wt %) compared to 1.1 wt % acetic acid content from another supplier used in the other coated cores R1, R2, R3, R5, and R6. A new batch of chitosan acetate (3 wt %) with approximately 2 wt % acetic acid was used in R7, with suitable results.

TABLE 8
Rumen-protected Methionine Capsule Formulations

| | FORMULATION | | | COAT- |
|---|---|---|---|---|
| | CORE (wt % of | COATING (wt % of | CHITOSAN | ING |
| # | formulation) | formulation) | ACETATE | [wt %] |
| R1 | Methionine: 70.7 | Fatty acid: 13.9 | Supplier #1, | 16.8 |
| | Lipids: 8.3 | Chitosan acetate: 2.2 | batch 1 | |
| | Lysine: 4.2 | Lecithin: 0.7 | | |
| R2 | Methionine: 70.9 | Fatty acid: 13.8 | Supplier #1, | 16.6 |
| | Lipids: 8.3 | Chitosan acetate: 2.5 | batch 1 | |
| | Lysine: 4.2 | Lecithin: 0.3 | | |
| R3 | Methionine: 70.2 | Fatty Acid: 14.4 | Supplier #1, | 17.4 |
| | Lipids: 8.3 | Chitosan acetate: 2.6 | batch 1 | |
| | Lysine: 4.1 | Lecithin: 0.3 | | |
| R4 | Methionine: 70.8 | Fatty Acid: 13.9 | Supplier #2, | 16.7 |
| | Lipids: 8.3 | Chitosan acetate: 2.5 | batch 1 | |
| | Lysine: 4.2 | Lecithin: 0.3 | | |
| R5 | Methionine: 69.3 | Fatty Acid: 15.4 | Supplier #1, | 18.5 |
| | Lipids: 8.2 | Chitosan acetate: 2.8 | batch 2 | |
| | Lysine: 4.1 | Lecithin: 0.7 | | |
| R6 | Methionine: 69.3 | Fatty Acid: 15.4 | Supplier #1, | 18.5 |
| | Lipids: 8.2 | Chitosan acetate: 2.4 | batch 2 | |
| | Lysine: 4.1 | Lecithin: 0.7 | | |
| R7 | Methionine: 71.6 | Fatty Acid: 12.7 | Supplier #2, | 15.8 |
| | Lipids: 8.4 | Chitosan acetate: 2.1 | batch 2 | |
| | Lysine: 4.2 | Lecithin: 1.0 | | |

The rumen stability of the capsules was evaluated in vitro using an assay referred to as the Buffer/Lipase Assay. To perform this assay, approximately 5 g of the capsules were suspended in 200 mL of 0.05 M buffer consisting of dihydrogen potassium phosphate, and dibasic sodium phosphate (Traceable to N.I.S.T, certified pH 7.00, Fisher Chemicals), and 0.4 g of Pancrelipase enzyme (24 USP units/mg, American Laboratories, Inc.), and then incubated in a water bath at 39° C. with gentle mixing (about 160 rpm). At specified time points, the percentage of methionine released from the product was measured. Results are provided in Table 9. These prototypes were made with the same stearic acid and lecithin, but chitosan acetate was from different suppliers as provided in Table 5. The assay was expected to show the stability of the encapsulates in a simulated rumen environment. It was observed that R4 showed very high methionine release, and thus failed as a suitable prototype. Investigation showed the chitosan acetate in R4 was supplied as liquid (3 wt % chitosan acetate), which had a high acetic acid content (6 wt %) compared to 1.1 wt % acetic acid from another supplier #1 used in the other coated cores R1, R2, R3, R5, and R6. A new batch of chitosan acetate (3 wt %) with approximately 2 wt % acetic acid was used in R7, with suitable results. Generally, a high percentage of acetic acid from approximately 2% to approximately 3% showed an increase in release, suggesting that less than 2 wt % acetic acid may improve rumen protection.

TABLE 9
Capsule in Buffer/Lipase Assay: Percent Methionine Released

| | % released | % released | % released | % released | % released |
|---|---|---|---|---|---|
| # | at 4 hours | at 8 hours | at 12 hours | at 16 hours | at 24 hours |
| R1 | 8 | 14 | 18 | — | 25 |
| R2 | 18 | 34 | 42 | 49 | 60 |
| R3 | 17 | 28 | 35 | 41 | 50 |
| R4 | 48 | 68 | 91 | 97 | 98 |
| R5 | 21 | 30 | 44 | 51 | 57 |
| R6 | 14 | 21 | 28 | 34 | 39 |
| R7 | 7 | 14 | 20 | 23 | 31 |

Next, methionine content was determined by the sodium thiosulfate-iodine method or iodometric titration. Briefly, a sample aliquot (approximately 5 mL) was mixed in a titration cup with exactly 10.0 mL of 0.1 N iodine standard (known to be in excess) in 100 mL of 0.75 M sodium phosphate buffer solution containing 5 g of dibasic sodium phosphate and 2 g of monobasic sodium phosphate, and 0.5 g of potassium iodide crystals. The mixture was capped and incubated in the dark for 30 minutes. The mixture was then auto-titrated against standard 0.1 N sodium thiosulfate solution to the endpoint determined potentiometrically. A blank containing only 10.0 mL of 0.1 N iodine standard was analyzed to determine the total volume of sodium thiosulfate required for complete reduction of iodine. The difference in the endpoint volumes (blank and sample aliquot) represents the volume of iodine consumed by methionine because the normality of sodium thiosulfate and iodine standards is the same. The percentage of methionine was calculated by converting the volume of iodine consumed to equivalent amount of methionine and comparing the result to the theoretical amount of methionine present in the sample. For calculation, 1.0 mL of 0.1 N sodium thiosulfate standard is equivalent to 7.461 mg of methionine.

Additionally, bioavailability was evaluated in vitro by measuring the percentage of sequential methionine release from a series of solutions. About 5 g of the capsules were suspended in 200 mL solutions which simulate the rumen, the abomasum, and the small intestine. Bioavailability is determined by subtracting the percent of methionine released at 16 hours in the bioavailability assay from the percent of methionine released at 8 hours in the bioavailability assay, which corresponds to the amount of methionine released in the abomasum and small intestine. This is referred to herein as “the bioavailability assay.”

Table 10 shows the release of methionine in a bioavailability assay over 24 hours from capsules R1-R7 produced by coating with a coating composition consisting of stearic acid, chitosan acetate, and lecithin. A pH-mediated high release of the prototypes R1-R3 and R5-R7 is clearly demonstrated. Indeed, R4 showed low bioavailability due to poor simulated rumen stability, most likely due to high level of residual acetic acid. Generally, residual acetic acid in chitosan acetate of less than 3% is important for improving rumen protection and bioavailability.

TABLE 10
Percent Methionine Released and Bioavailability

| | 4 | 8 | 10 | 13 | 16 | |
|---|---|---|---|---|---|---|
| # | hours | hours | hours | hours | hours | Bioavailability |
| R1 | 8 | 14 | 53 | 85 | 100 | 86 |
| R2 | 18 | 31 | 61 | 88 | 100 | 69 |
| R3 | 15 | 23 | 56 | 85 | 96 | 73 |
| R4 | 41 | 63 | 81 | 99 | 100 | 37 |
| R5 | 19 | 31 | 58 | 89 | 97 | 67 |
| R6 | 13 | 21 | 49 | 85 | 94 | 73 |
| R7 | 10 | 16 | 52 | 90 | 94 | 77 |

Release of methionine in the bioavailability assay was determined over 16 hours from prototypes produced by coating with stearic acid/chitosan acetate (CA) and lecithin aqueous dispersions at different levels as shown by prototypes R1-R7 in Table 6. A pH-mediated high release of the prototypes R1-R3 and R5-R7 is clearly demonstrated in the bioavailability assay (see Table 7). R4 showed low bioavailability due to poor simulated rumen stability, most likely due to high levels of residual acetic acid.

### Example 9: Comparator Capsule Formulation Including Chitosan

Capsules were made to observe rumen stability and bioavailability differences between capsules formulated with chitosan and capsules formulated with chitosan acetate. The capsules had the compositions shown in Table 11:

TABLE 11
Compositions of comparator capsules.

| FORMULATION | | |
|---|---|---|
| CORE (wt % of | COATING (wt % of | COATING |
| formulation) | formulation) | [wt %] |
| Methionine: 73.2 | Fatty acid: 11.7 | 13.9 |
| Lipids: 8.6 | Chitosan: 1.1 | |
| Lysine: 4.3 | Lecithin: 1.1 | |
| Methionine: 73.2 | Fatty acid: 11.7 | 13.9 |
| Lipids: 8.6 | Chitosan acetate: 1.1 | |
| Lysine: 4.3 | Lecithin: 1.1 | |

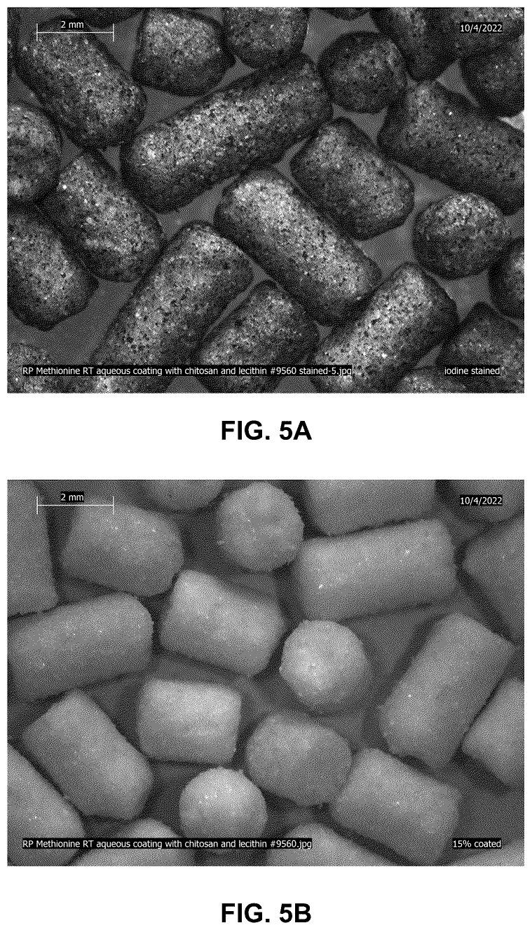

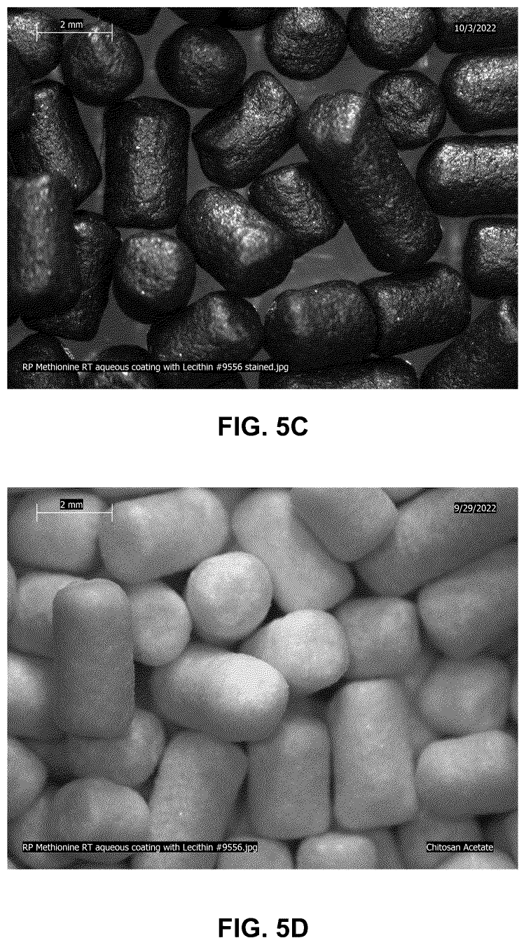

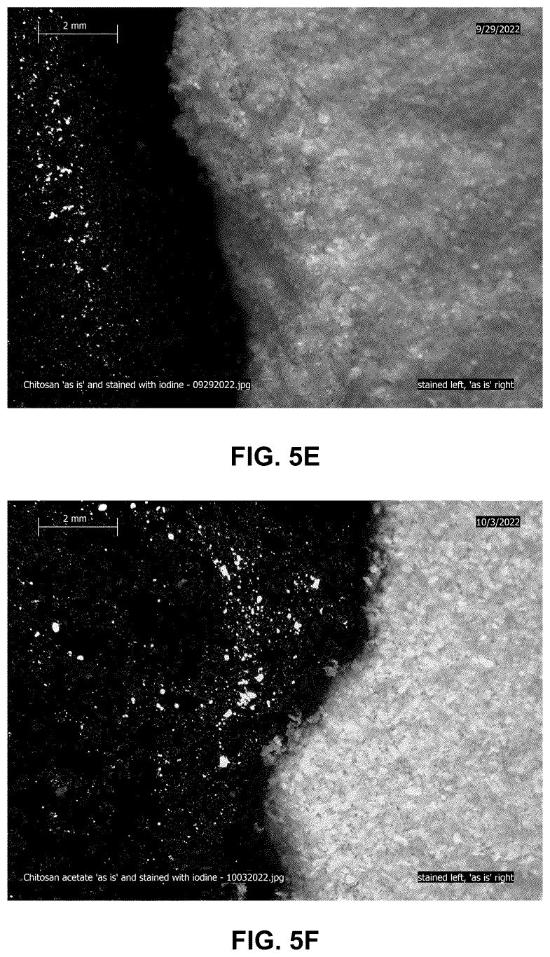

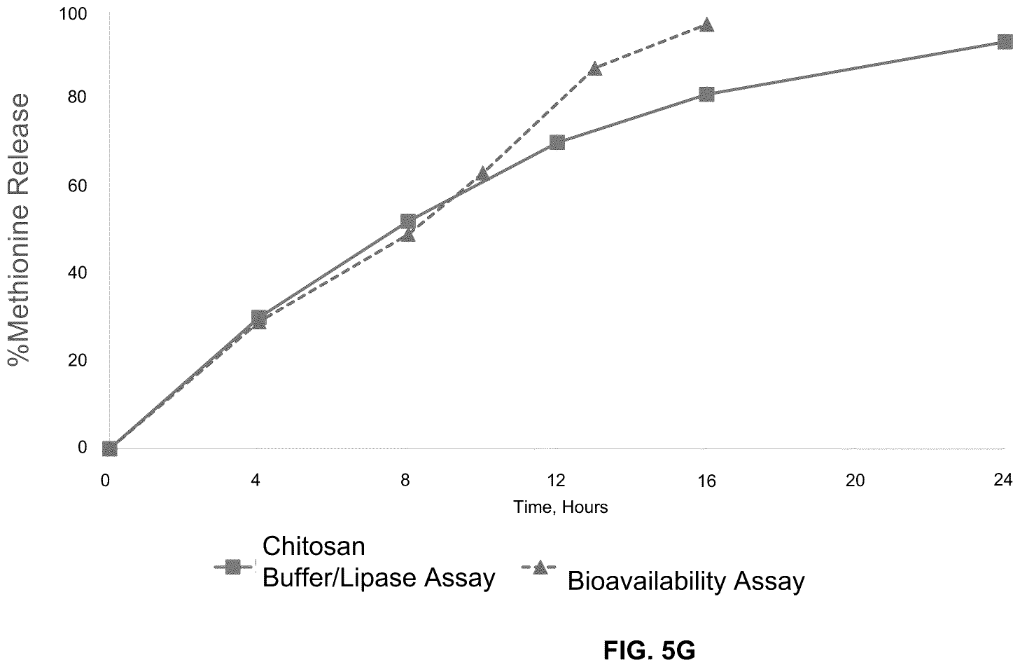

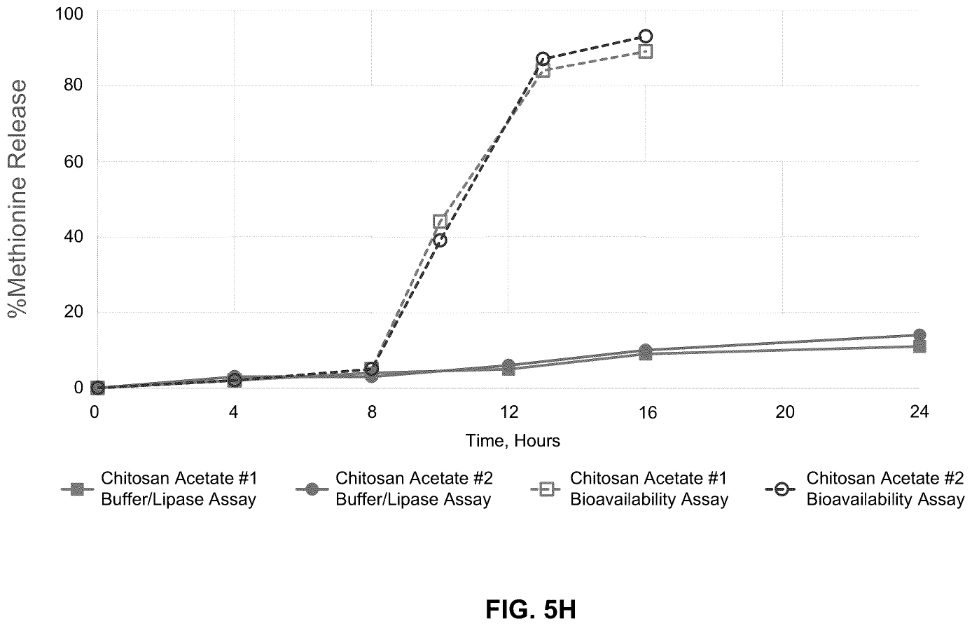

FIGS. 5A-5D show capsules made using chitosan (5A & 5B) and chitosan acetate (5C & 5D). The capsules were stained with iodine, which causes the chitosan and the chitosan acetate to turn black, as shown in FIGS. 5E and 5F, respectively. As can be seen in FIG. 5A, the chitosan is dispersed in small discrete particles on the surface of the capsule; whereas, in FIG. 5C, the chitosan acetate is evenly distributed over the entire capsule.

The composition containing chitosan was tested for rumen stability using the Buffer/Lipase Assay and for bioavailability using the Bioavailability Assay described herein. FIG. 5G shows the percent methionine released as a function of time in both the buffer/lipase assay and the bioavailability assay. It can be seen from FIG. 5G that the about 50% of the methionine is lost in the rumen after about 8 hours in both the buffer/lipase assay and in the bioavailability assay. The total bioavailability of the chitosan formulation was approximately 48%.

For comparison, the rumen stability and bioavailability of two rumen-protected compositions containing chitosan acetate were tested as well, shown in FIG. 5H. It can be seen from FIG. 5H that the rumen stability and bioavailability are dramatically improved compared with the coating comprising chitosan. The total bioavailability of the chitosan acetate compositions shown here was 84% and 88%.

### Example 10: Additional Optimized Capsule Formulations

Capsules with varying amounts of stearic acid, chitosan acetate and lecithin were used to coat several methionine cores using a fluid bed system (see Table 12). The chitosan acetate to lecithin ratio (CA/LE) for each capsule formulation is provided for comparison. The lipids in the core composition comprised a hydrogenated mixture of fatty acids and hydrogenated vegetable oils. The fatty acid included a blend of stearic acid and palmitic acid. The prototypes were tested for bioavailability and release as in Example 4 to determine the more suitable compositions.

TABLE 12
Additional Capsule Formulations

| | FORMULATION | | % | CHITOSAN |
|---|---|---|---|---|
| | CORE (wt % of | COATING (wt % of | COAT- | ACETATE/ |
| # | total formulation) | total formulation) | ING | LECITHIN |
| R8 | Methionine: 71.6 | Fatty acid: 12.6 | 15.8 | 4.0 |
| | Lipids: 8.4 | Chitosan acetate: 2.5 | | |
| | Lysine: 4.2 | Lecithin: 0.6 | | |
| R9 | Methionine: 69.8 | Fatty acid: 14.0 | 17.9 | 4.5 |
| | Lipids: 8.2 | Chitosan acetate: 3.1 | | |
| | Lysine: 4.1 | Lecithin: 0.8 | | |
| R10 | Methionine: 70.7 | Fatty acid: 12.9 | 16.9 | 5.0 |
| | Lipids: 8.3 | Chitosan acetate: 3.3 | | |
| | Lysine: 4.2 | Lecithin: 0.7 | | |
| R11 | Methionine: 68.6 | Fatty acid: 15.5 | 19.3 | 2.3 |
| | Lipids: 8.1 | Chitosan acetate: 2.2 | | |
| | Lysine: 4.0 | Lecithin: 1.2 | | |
| R12 | Methionine: 71.6 | Fatty acid: 12.7 | 15.8 | 2.1 |
| | Lipids: 8.4 | Chitosan acetate: 2.1 | | |
| | Lysine: 4.2 | Lecithin: 1.0 | | |
| R13 | Methionine: 72.1 | Fatty acid: 11.9 | 15.2 | 1.8 |
| | Lipids: 8.5 | Chitosan acetate: 2.1 | | |
| | Lysine: 4.2 | Lecithin: 1.2 | | |
| R14 | Methionine: 70.4 | Fatty acid: 13.4 | 17.2 | 1.8 |
| | Lipids: 8.3 | Chitosan acetate: 2.4 | | |
| | Lysine: 4.1 | Lecithin: 1.4 | | |
| R15 | Methionine: 69.2 | Fatty acid: 14.5 | 18.6 | 1.7 |
| | Lipids: 8.1 | Chitosan acetate: 2.6 | | |
| | Lysine: 4.1 | Lecithin: 1.5 | | |
| R16 | Methionine: 72.8 | Fatty acid: 11.5 | 14.4 | 1.5 |
| | Lipids: 8.6 | Chitosan acetate: 1.7 | | |
| | Lysine: 4.3 | Lecithin: 1.2 | | |
| R17 | Methionine: 69.0 | Fatty acid: 14.2 | 18.8 | 1.4 |
| | Lipids: 8.1 | Chitosan acetate: 2.6 | | |
| | Lysine: 4.1 | Lecithin: 1.9 | | |
| R18 | Methionine: 69.3 | Fatty acid: 15.2 | 18.5 | 1.3 |
| | Lipids: 8.1 | Chitosan acetate: 1.9 | | |
| | Lysine: 4.1 | Lecithin: 1.5 | | |
| R19 | Methionine: 70.4 | Fatty acid: 14.4 | 17.2 | 1.0 |
| | Lipids: 8.3 | Chitosan acetate: 1.4 | | |
| | Lysine: 4.1 | Lecithin: 1.4 | | |
| R20 | Methionine: 72.8 | Fatty acid: 11.8 | 14.3 | 0.8 |
| | Lipids: 8.6 | Chitosan acetate: 1.1 | | |
| | Lysine: 4.3 | Lecithin: 1.4 | | |
| R21 | Methionine: 70.2 | Fatty acid: 13.9 | 17.4 | 0.7 |
| | Lipids: 8.3 | Chitosan acetate: 1.4 | | |
| | Lysine: 4.1 | Lecithin: 2.1 | | |
| R22 | Methionine: 70.3 | Fatty acid: 14.0 | 17.3 | 0.7 |
| | Lipids: 8.3 | Chitosan acetate: 1.4 | | |
| | Lysine: 4.1 | Lecithin: 1.9 | | |
| R23 | Methionine: 68.3 | Fatty acid: 15.9 | 19.7 | 0.7 |
| | Lipids: 8.0 | Chitosan acetate: 1.6 | | |
| | Lysine: 4.0 | Lecithin: 2.2 | | |

Table 13 shows in vitro rumen stability of capsules with varying coating compositions. Data shows rumen stability is low for R8 to R10 suggesting the coating is unstable when the coating level of at least 15% consists of chitosan acetate to lecithin ratio of at least 4:1. However for coating levels consisting of chitosan acetate to lecithin ratios of at least 3:1 or lower (R11-R23), rumen stability is significantly improved.

TABLE 13
Buffer/Lipase Assay for Optimal Coating Composition:
Percent Methionine Released and Rumen Stability

| | % Release | % Release | % Release | % Release | % Release |
|---|---|---|---|---|---|
| # | at 4 hours | at 8 hours | at 12 hours | at 16 hours | at 24 hours |
| R8 | 17 | 29 | 38 | 40 | 51 |
| R9 | 18 | 31 | 39 | 42 | 56 |
| R10 | 21 | 35 | 46 | 52 | 66 |
| R11 | 6 | 13 | 15 | 17 | 24 |
| R12 | 5 | 12 | 13 | 15 | 20 |
| R13 | 2 | 8 | 9 | 9 | 19 |
| R14 | 7 | 14 | 20 | 23 | 30 |
| R15 | 1 | 7 | 8 | 13 | 16 |
| R16 | 1 | 6 | 7 | 12 | 14 |
| R17 | 3 | 7 | 9 | 13 | 16 |
| R18 | 0 | 5 | 6 | 11 | 14 |
| R19 | 2 | 7 | 8 | 12 | 18 |
| R20 | 5 | 10 | 16 | 23 | 28 |
| R21 | 5 | 12 | 18 | 26 | 34 |
| R22 | 4 | 7 | 15 | 19 | 23 |
| R23 | 2 | 5 | 8 | 11 | 15 |

Table 14 shows release of methionine in the bioavailability assay over 16 hours from prototypes produced by various coating compositions to determine in vitro bioavailability. As stated earlier, low rumen protection of R8-R10 leads to low bioavailability, however, improved rumen protection of R11-R23 leads to significantly high bioavailability.

TABLE 14
Performance Data for Optimal Coating Compositions -
Percentage Methionine Released and Bioavailability

| | % Released at | % Released at | % Released at | % Released at | % Released at | |
|---|---|---|---|---|---|---|
| # | 4 hours | 8 hours | 10 hours | 13 hours | 16 hours | Bioavailability |
| R8 | 18 | 28 | 55 | 82 | 92 | 64 |
| R9 | 23 | 35 | 66 | 89 | 100 | 65 |
| R10 | 24 | 37 | 65 | 92 | 100 | 63 |
| R11 | 6 | 9 | 44 | 90 | 96 | 87 |
| R12 | 2 | 8 | 53 | 93 | 100 | 91 |
| R13 | 3 | 6 | 33 | 71 | 84 | 78 |
| R14 | 10 | 16 | 52 | 90 | 94 | 77 |
| R15 | 5 | 8 | 53 | 88 | 93 | 85 |
| R16 | 5 | 6 | 35 | 73 | 82 | 76 |
| R17 | 4 | 7 | 50 | 85 | 89 | 82 |
| R18 | 4 | 5 | 35 | 79 | 88 | 82 |
| R19 | 3 | 6 | 41 | 87 | 91 | 85 |
| R20 | 4 | 10 | 51 | 84 | 84 | 74 |
| R21 | 4 | 13 | 57 | 86 | 85 | 72 |
| R22 | 0 | 8 | 40 | 78 | 83 | 76 |
| R23 | 0 | 5 | 40 | 86 | 91 | 86 |

### Example 11: Using Stable Isotope Methodology to Establish Rumen-Protected Methionine Bioavailability

Accurate bioavailability assessment of rumen-protected nutrients is required for precise diet formulation. Inaccurate assessments lead to flawed assumptions, resulting in underperforming animals and higher ration costs. Characterizing these types of products in terms of absorption and digestibility is challenging, and this information often relies on indirect methods. Numerous in vitro, in situ and in vivo methods of assessing the ruminal disappearance and intestinal availability of rumen-protected amino acids (RP-AA) have been developed; however, they all have limitations. Additionally, bioavailability assessment of rumen-protected methionine products has been hampered due to the lack of a reliable assessment methodology. The stable isotope technique has recently emerged as a consistent and extremely accurate method for evaluating methionine bioavailability. The new technique is an in vivo methodology that can accurately determine the plasma appearance of a variety of ingredients by measuring and modeling plasma AA isotopic enrichment data. This is a preferred methodology to test the bioavailability of RP-AA products due to the sensitivity of the technique. Also, ingredients of interest are mixed in the diet, fed to the cows and there is no need to compare results back to an abomasal infusion of unprotected AA.

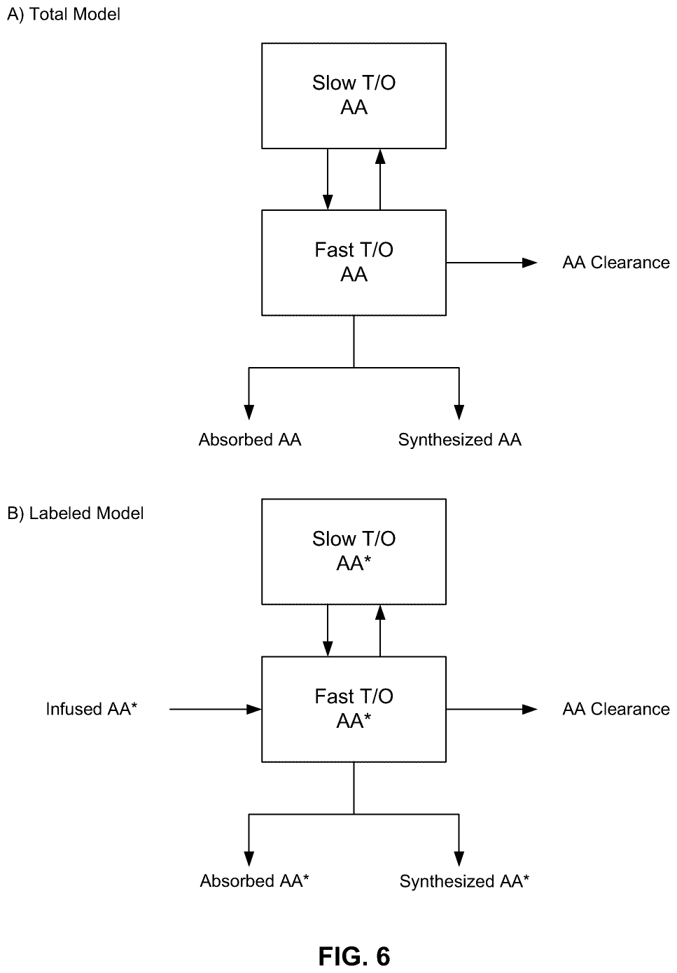

In brief, the stable isotope technique employs a jugular infusion of isotopically labeled amino acid (AA) that act as tracers in an in vivo system. Isotopic enrichment of plasma AA in response to the jugular infusion is measured and modeled in a 4-pool model (which encompasses protein turnover pools; see FIG. 6) to derive AA plasma entry rates in animals fed diets containing a nutrient of interest. This is a noninvasive, in vivo methodology that provides accurate and precise assessments of AA absorption for feed ingredients in a more typical feeding environment (i.e. no need for ruminally or intestinally cannulated animals, abomasal infusions, pulse doses, etc.), making it a more preferred, unbiased testing methodology for RP-AA products.

The objective of this experiment was to determine the methionine plasma appearance and bioavailability of a rumen-protected methionine product (Control product #1,), utilizing the stable isotope technique. Control product #1 is a rumen-protected methionine produced by precision release technology. The composition is composed of not less than 68 wt % of DL methionine, not less than 2 wt % L-lysine hydrochloride, and 10-30 wt % other ingredients including hydrogenated vegetable oil. Control product #1 core is produced through a microencapsulation process using layers of lipids around a true solid core for precision release of methionine.

The experiment was conducted as a 6×7 Latin square design with 6 cows and 7 periods each lasting 10 days in length. All cows were fed one common base diet predicted to be sufficient in AA and energy supply for a lactating cow producing 53 lbs of milk. The trial consisted of seven different AA treatments, but only the description and results for control product #1 treatment are provided here. Control product #1 was supplemented in the ration at 65 g/cow/d. All RP-AA products were top dressed and hand mixed within the top layer of feed once per day from days 1 through 7, but from days 8 through 10 the products were mixed into the base diet using a Data Ranger.

On days 9 and 10, all cows were fed every 2 hours to achieve steady state conditions and minimize variations in AA absorption. On day 10 of each period, cows received an 8 hr jugular infusion of a complete mixture of isotopically labeled AA. Blood samples were taken before and during the infusion and were analyzed for plasma AA isotopic enrichment. The data was then modeled using a dynamic 4-pool model to derive diet amino acid entry rates. The resulting data was further analyzed in a regression analysis to determine AA entry rates into the plasma for each AA product tested. A schematic representation of this model is shown in FIG. 6.

Table 15 reports the plasma appearance and bioavailability estimates of control product #1 from the isotope experiment, as well as the plasma appearance and bioavailability of a composition of the present disclosure comprising 74 wt % methionine and 13 wt % coating (Capsule #1 as prepared in Example 7). Plasma appearance is calculated as the grams of methionine absorbed into blood per gram of methionine fed. Bioavailability was calculated as previously described and corrected for 7% loss during first pass. Roughly 51% of the methionine content of control product #1 by-passed the rumen and was absorbed into the plasma. However, there is some initial use of AA by the intestinal tissue during absorption and first passage through the liver that is considered bioavailable to the cow but is not captured in the plasma appearance value. If it is assumed that 7% of AA are utilized during first pass use, control product #1 has a methionine bioavailability of 55%. To summarize, control product #1 has a methionine plasma appearance value of 51% and a calculated methionine bioavailability of 55%, and capsule #1 has a plasma appearance value of 82.3% and a methionine bioavailability of 88.6%.

TABLE 15
Plasma Appearance and Bioavailability
Results from the Isotope Experiment

| | Plasma Appearance | Bioavailability |
|---|---|---|
| Control product #1 | 51.2% | 55.0% |
| Capsule #1 | 82.3% | 88.6% |

The results show that the Rumen-protected methionine formulation shows improved bioavailability over Control product #1 in an in vivo study, thus making it a better choice compared to available products.

### Example 12: Determination of Methionine Bioavailability of Capsules Using the Abomasal Pulse Dose Method

Ruminally protected amino acids (RP-AA) are used to supplement ruminant diets to achieve a better balance of amino acids (AA). The abomasal pulse dose technique is one method designed to determine how much of the RP-AA bypass the rumen and are truly absorbed in the small intestine. By ensuring that the RP-AA are functioning as intended, these products can be utilized to improve our understanding of amino acid requirements in cattle. Improved understanding of amino acid requirements will allow producers to design and feed diets to cows that more precisely match the cows' amino acid needs. This balance will lead to improved nitrogen efficiency, milk production and milk components.

Ruminally protected AA products can be assessed for ruminal protection and small intestine bioavailability using blood appearance responses after an abomasal bolus and then calculating the area under the curve (AUC). The abomasal bolus method makes use of the rise in plasma free AA concentrations after a bolus of a RP-AA product to determine relative appearance which is compared to the relative appearance of an abomasally infused unprotected AA. This methodology of calculating the AUC is described in Graulet et al., 2005.

Products were tested in each of 2 nonlactating, rumen cannulated cows in a cross-over design. No two cows received the same treatment on the same day and each day was considered its own period. The cows were fed a standard daily ration for high lactation cows (16.5% CP to ensure that the AA supply meets or exceeds requirements) 6×/day (every 4 h) for 24 h prior to the trial and throughout the trial. Feeding was restricted to 95% of ad libitum intake starting 12 h prior to the first period to help reduce diurnal variation in plasma AA.

Treatments (RP-AA product and unprotected AA) were designed to provide equivalent doses of AA. For each infusion, samples of the RP-AA product were evenly distributed into four, 10×20 cm dacron bags (4 bags per product, 50 μm porosity, Ankom) and were incubated in the rumen of the cannulated cow for 8 hours prior to abomasal bolus. Additional capsule samples of the RP-AA products tested were also incubated in the rumen in 10×20 cm dacron bags (1 bag per product, 50 μm porosity, Ankom) for 8 hours to determine ruminal disappearance of AA nitrogen. The dacron bags were placed together in one laundry bag and suspended in the rumen. Residue remaining after 8 hours was collected from the bags and was infused into the abomasum. Abomasal infusion lines were constructed from small-bore tygon tubing with a tygon flange attached to the distal end. The lines were introduced into the abomasum via the reticular, omasal orifice as previously described by Spires et al. (1975). Unprotected AA was bolused into the abomasum without prior rumen incubation as the control treatment. The bolus dosing required between 10-15 minutes using less than 2 L of water. After the infusion, the line was flushed with an additional 100 mL of water to ensure complete delivery of the dose.

Following rumen incubation, the additional capsule samples used to determine N degradation in the rumen were rinsed 3× in fresh cold water, excess moisture was removed using a salad spinner, stored at −20° C., and subsequently freeze dried. Dried residue and a subsample of the source material were ground and analyzed for N content by combustion via Vario El Cube (Elementar, Germany).

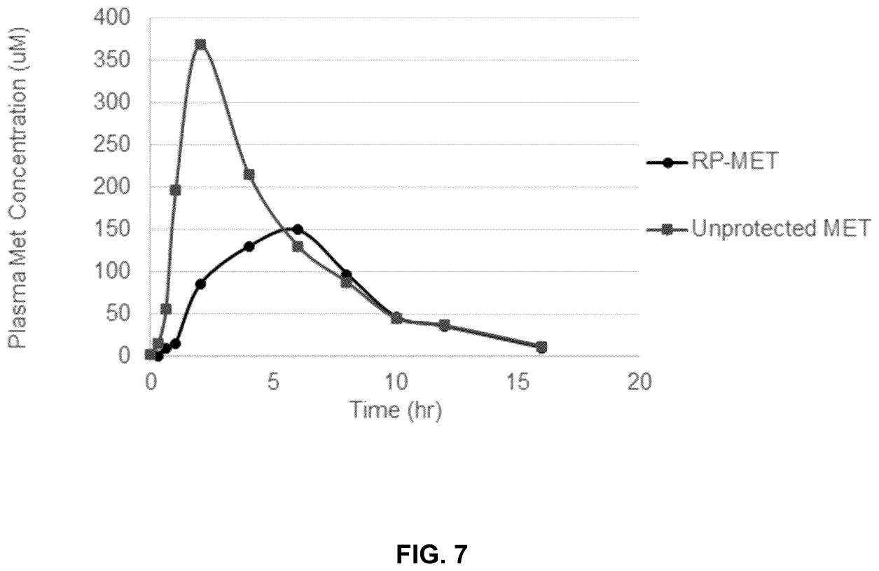

Blood was sampled from an indwelling jugular catheter at −4, −2, 0, 0.33, 0.67, 1, 2, 4, 6, 8, 10, 12, and 16 h relative to the completion of the bolus. Plasma was prepared from the collected blood by centrifugation (1665 RCF for 12 min) and stored in polypropylene tubes at −20° C. until further analysis. Plasma samples were deproteinized using sulfosalicylic acid (SSA; 50% SSA v/v) and centrifugation, desalted by ion exchange (BioRad Resin AG 50-X*, 100-200 mesh), and eluted using ammonium hydroxide (2N) into silanized glassware as described by Calder et al. (1999). Desalted samples were freeze dried and subsequently derivatized using Acetonitrile and N-tert-butyldimethylsilyl-N-methyltrifluoroacetamide (MTBSTFA). Samples were then analyzed for AA concentrations by gas chromatography-mass spectrometry using isotope dilution as described by Calder et al., 1999. Blood AA concentrations (μM) are determined for each plasma sample for both the unprotected infused AA treatment as well as the RP-AA products (see FIG. 7). The area under the plasma concentration curve for the encapsulated product and unprotected AA were calculated using the trapezoid rule. A bioavailability value was determined as a ratio of the encapsulated area over the unprotected area with corrections for any differences in the amount of nitrogen infused as well as baseline plasma amino acid content.

Control product #1 as provided in Example 10 (74% methionine with a coating consisting of a lipid blend) was tested using the previously described abomasal pulse dose method where control product #2 (75% methionine) was used as the control treatment. Unlike control product #1, control product #2 is a rumen-protected methionine coated with a coating composition containing a synthetic pH-sensitive polymer. The composition is composed of greater than 70 wt % of DL methionine, less than 25 wt % stearic acid, less than 2 wt % ethyl cellulose, and 2-3 wt % other ingredients including the pH sensitive polymer, copolymer poly (2-vinylpyridine-co-styrene). To prepare control product #2, a hot coating emulsion composed of the ingredients mentioned is prepared and maintained at 90° C. before applying onto methionine/lysine pellets through a Uniglatt equipped with a Wurster insert, as described in EP0462015B1, and U.S. Pat. No. 5,225,238, the entireties of which are incorporated by reference herein.

Treatments were designed so that control product #1 and #2 provided equivalent methionine doses of 40 g (54.05 g of #1 or 53.33 g of #2). Results of the experiment for control product #1 are shown in Table 15. It was determined that after an 8 h ruminal incubation, 70% of the methionine was remaining and available for small intestine digestion. Plasma methionine concentrations yielded an AUC of 1162, and after comparing to plasma methionine appearance of control product #2, a bioavailability value of 42.7% was calculated. Intestinal availability and metabolizable methionine were calculated based on the bioavailability value, known methionine content and/or rumen bypass of control product #1.

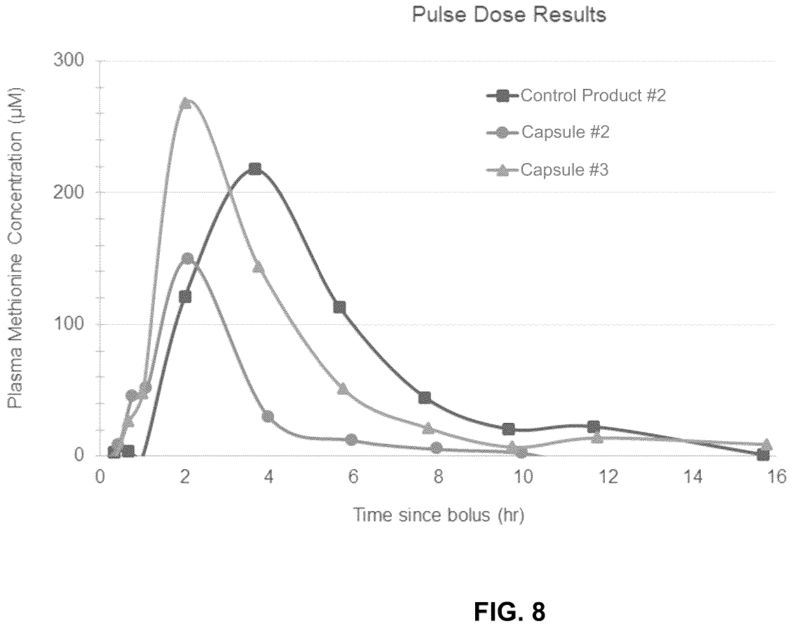

Control product #1 was then tested using the abomasal pulse dose method, along with two compositions (Capsule #2, Capsule #3) of the present disclosure. Capsule #2 has the composition of formula R26 described in Table 17. Capsule #3 has the composition of formula R12 described in Table 12. The results are shown in Table 16 and in FIG. 8. Results show that the triggered release composition of capsule #3 is able to achieve a high rumen bypass (80%) with a 100% relative bioavailability (i.e., 80% total bioavailability).

TABLE 16
Abomasal pulse dose results

| | Rumen Bypass (%) | Total Bioavailability (%) |
|---|---|---|
| Capsule #2 | 53 | 40 |
| Capsule #3 | 80 | 80 |
| Control product #1 | 70 | 34 |

### Example 13: Coating Compositions with Tween 80 or Lecithin

Next, coatings with Tween 80 or Lecithin were compared to determine which composition will provide best desired performance attributes of high rumen protection and high bioavailability. The lipids in the core composition comprised a mixture of fatty acids and hydrogenated vegetable oils. The fatty acid included a blend of stearic acid and palmitic acid. The results are shown in Table 17.

TABLE 17
Coating Compositions Containing Tween 80 or Lecithin

| | FORMULATION | | |
|---|---|---|---|
| | CORE (wt % of | COATING (wt % of | wt % of |
| # | total formulation) | total formulation) | COATING |
| R24 | Methionine: 68.3 | Fatty acid: 16.5 | 20 |
| | Lipids: 8.0 | Chitosan acetate: 1.6 | |
| | Lysine: 4.0 | Tween 80: 1.6 | |
| R25 | Methionine: 68.3 | Fatty acid: 15.2 | 18 |
| | Lipids: 8.4 | Chitosan acetate: 1.4 | |
| | Lysine: 4.0 | Tween 80: 1.4 | |
| R26 | Methionine: 71.1 | Fatty acid: 13.8 | 16 |
| | Lipids: 8.4 | Chitosan acetate: 1.3 | |
| | Lysine: 4.2 | Tween 80: 1.3 | |
| R27 | Methionine: 70.8 | Fatty acid: 14.0 | 17 |
| | Lipids: 8.3 | Chitosan acetate: 1.3 | |
| | Lysine: 4.2 | Tween 80: 1.3 | |
| R28 | Methionine: 69.7 | Fatty acid: 15.1 | 18 |
| | Lipids: 8.2 | Chitosan acetate: 1.4 | |
| | Lysine: 4.1 | Lecithin: 1.4 | |
| R29 | Methionine: 72.1 | Fatty acid: 11.9 | 17 |
| | Lipids: 8.5 | Chitosan acetate: 2.1 | |
| | Lysine: 4.2 | Lecithin: 1.2 | |
| R30 | Methionine: 70.4 | Fatty acid: 13.4 | 18 |
| | Lipids: 8.3 | Chitosan acetate: 2.4 | |
| | Lysine: 4.1 | Lecithin: 1.4 | |
| R31 | Methionine: 69.2 | Fatty acid: 14.5 | 17 |
| | Lipids: 8.1 | Chitosan acetate: 2.6 | |
| | Lysine: 4.1 | Lecithin: 1.5 | |
| R32 | Methionine: 72.8 | Fatty acid: 11.5 | 17 |
| | Lipids: 8.6 | Chitosan acetate: 1.7 | |
| | Lysine: 4.3 | Lecithin: 1.2 | |

The prototypes were tested for performance to determine the most suitable composition as shown in Table 18. Results show release of methionine in the bioavailability assay over 16 hours from prototypes produced by coating compositions containing Tween 80 or lecithin to determine in vitro bioavailability. The data shows coating compositions containing Tween 80 had higher release in the rumen than those with lecithin. Additionally, bioavailability is superior in coating compositions containing lecithin than compositions containing Tween 80.

TABLE 18
Bioavailability Assay - Percent Methionine Released and Bioavailability

| | % Release at | % Release at | % Release at | % Release at | % Release at | |
|---|---|---|---|---|---|---|
| # | 4 hours | 8 hours | 10 hours | 13 hours | 16 hours | Bioavailability |
| R24 | 5 | 10 | 53 | 85 | 89 | 80 |
| R25 | 9 | 10 | 43 | 77 | 84 | 75 |
| R26 | 9 | 17 | 76 | 99 | 99 | 82 |
| R27 | 9 | 17 | 73 | 100 | 100 | 83 |
| R28 | 4 | 6 | 57 | 94 | 96 | 91 |
| R29 | 3 | 6 | 55 | 98 | 98 | 93 |
| R30 | 3 | 5 | 56 | 97 | 98 | 93 |
| R31 | 1 | 4 | 47 | 82 | 90 | 87 |
| R32 | 1 | 3 | 43 | 91 | 93 | 91 |

### Example 14: Shelf-life Stability

Shelf-life stability of encapsulated prototypes was determined using capsules of methionine produced previously and stored at ambient temperature lab environment. The formulations were re-tested for rumen stability after 16 months (Table 12: R11, R12, and R13) using a Buffer/Lipase Assay. The assay data collected initially and after 16 months showed no significant effect on shelf-life stability as shown in Table 19. Thus, the formulations were found to be stable at least over the tested duration of 16 months.

TABLE 19
Shelf-life Stability

| | | | | | |
|---|---|---|---|---|---|
| | % Release | % Release | % Release | % Release | % Release |
| # | at 4 hours | at 8 hours | at 12 hours | at 16 hours | at 24 hours |

Initial test (April 2020)

| | | | | | |
|---|---|---|---|---|---|
| R12 | 5 | 12 | 13 | 15 | 20 |
| R13 | 2 | 8 | 9 | 9 | 19 |
| R14 | 7 | 14 | 20 | 23 | 30 |

16-Month test (August 2021)

| | | | | | |
|---|---|---|---|---|---|
| R12 | 4 | 8 | 10 | 12 | 18 |
| R13 | 4 | 8 | 10 | 13 | 17 |
| R14 | 7 | 15 | 18 | 23 | 29 |

### Example 15: Mineral Mix Stability

Capsule prototypes were mixed with minerals to simulate expected damage during handling in feed mills. Approximately 1.0 kg of capsules were mixed with 45.4 kg of mineral mix granules and blended in mortar operated paddle/ribbon mixer vessel for 3 min. The final concentration of the capsules in the minerals mix was approximately 2.2 wt %. Each mixed material was sieved through US mesh #8, #10, #14, and #16 to isolate the capsules from the mineral mix granules; most capsules were collected on the 1400 μm size. The capsules were tested for in vitro mineral stability using the Buffer/Lipase assay. Results as provided in Table 20 show that the capsules are stable, suggesting the impact of minerals on coating integrity is minimal.

TABLE 20
Mineral Mix Stability: Percent Methionine
Released and Rumen Stability

| | % Re- | % Re- | % Re- | % Re- | % Re- |
|---|---|---|---|---|---|
| | lease | lease | lease | lease | lease |
| | at 4 | at 8 | at 12 | at 16 | at 24 |
| Treatment | hours | hours | hours | hours | hours |
| Capsule, control, | 4 | 9 | 13 | 18 | 28 |
| not mixed | | | | | |
| Capsule, mixed | 3 | 8 | 13 | 17 | 28 |
| batch 1 | | | | | |
| Capsule, mixed | 2 | 8 | 12 | 16 | 29 |
| batch 2 | | | | | |

### Example 16: Coating Methionine Cores Using a Fully Perforated Pan Coater

Additional coating methods were tested using the composition prepared as in Example 8. The coating methodology in Example 8 was replaced with a method using a fully perforated pan coater as provided. About 3.40 kg of cylindrical methionine cores (approximately 2 mm in diameter) were charged to a 6 L fully perforated pan coater conditioned to 40° C. ready for coating. The coating process was operated with the following conditions: inlet temp: −65° C., exhaust temp: −33-34° C., pan speed: 24 rpm, process air flow: 50 CFM, atomization air pressure: 14 psi, and pattern air pressure: 16 psi, pump speed: 18-20 rpm (16-17 g/min) for approximately 180 min.

About 3.85 kg of capsule was produced, with about 13.2% coating weight (i.e. (3.85−3.40)/3.40=13.2% weight gain). Activity of methionine coated by this process was approximately 74% methionine (i.e. core is 85% methionine×[100%−13.2%]coating)=(85%×86.8%)=73.8% methionine).

### Example 17: Preparation and Testing of New Compositions Using Perforated Pan Coater

New formulations were tested using the perforated pan coater for the coating procedures. The formulations were prepared essentially as in Example 8, but with slightly different compositions as provided in Table 21.

TABLE 21
Pan Coated Formulations

| | FORMULATION | | |
|---|---|---|---|
| | CORE (wt % of | COATING (wt % of | wt % of |
| # | total formulation) | total formulation) | COATING |
| P1 | Methionine: 71.2 | Fatty acid: 13.4 | 16 |
| | Lipids: 8.4 | Chitosan acetate: 1.3 | |
| | Lysine: 4.2 | Lecithin: 1.3 | |
| P2 | Methionine: 70.6 | Fatty acid: 14.3 | 17 |
| | Lipids: 8.3 | Chitosan acetate: 1.4 | |
| | Lysine: 4.2 | Lecithin: 1.4 | |
| P3 | Methionine: 67.2 | Fatty acid: 17.6 | 21 |
| | Lipids: 7.9 | Chitosan acetate: 1.7 | |
| | Lysine: 4.0 | Lecithin: 1.7 | |
| P4 | Methionine: 67.2 | Fatty acid: 17.6 | 21 |
| | Lipids: 7.9 | Chitosan acetate: 1.7 | |
| | Lysine: 4.0 | Lecithin: 1.7 | |
| P5 | Methionine: 74.0 | Fatty acid: 10.9 | 13 |
| | Lipids: 8.7 | Chitosan acetate: 1.0 | |
| | Lysine: 4.4 | Lecithin: 1.0 | |
| P6 | Methionine: 73.1 | Fatty acid: 11.8 | 14 |
| | Lipids: 8.6 | Chitosan acetate: 1.1 | |
| | Lysine: 4.3 | Lecithin: 1.1 | |
| P7 | Methionine: 71.2 | Fatty acid: 13.4 | 16 |
| | Lipids: 8.4 | Chitosan acetate: 1.3 | |
| | Lysine: 4.2 | Lecithin: 1.3 | |

The formulations were tested for rumen stability as described in Example 6 using the Buffer % Lipase assay. Results of the assay are provided in Table 22. Release rate varied greatly between formulations, however all formulations exhibited instability. Capsule prototypes had less than 1% moisture at the end of the batch except for P3 and P4, which had final moisture percent of at least 1%, suggesting high coating level and moisture content of at least 1% may cause coating instability.

TABLE 22
Buffer/Lipase Assay for Coating Composition: Percent
Methionine Released and Rumen Stability.

| | % Release | % Release | % Release | % Release | % Release |
|---|---|---|---|---|---|
| # | at 4 hours | at 8 hours | at 12 hours | at 16 hours | at 24 hours |
| P1 | 3 | 8 | 15 | 17 | 28 |
| P2 | 14 | 28 | 42 | 52 | 69 |
| P3 | 14 | 27 | 43 | 57 | 77 |
| P4 | 13 | 25 | 40 | 51 | 70 |
| P5 | 4 | 3 | 7 | 11 | 15 |
| P6 | 4 | 5 | 7 | 13 | 16 |
| P7 | 9 | 16 | 25 | 36 | 52 |

Next, pan coated capsules were subjected to at least 3 days of curing at 45° C. The cured capsules showed higher rumen stability compared to the uncured capsules, as shown in Table 23. The bioavailability of the cured capsules is shown in Table 24.

TABLE 23
Buffer/Lipase Assay for Coating Composition: Percent Methionine
Released And Rumen Stability (After Curing).

| | % Release | % Release | % Release | % Release | % Release |
|---|---|---|---|---|---|
| # | at 4 hours | at 8 hours | at 12 hours | at 16 hours | at 24 hours |
| P1 | 3 | 8 | 15 | 17 | 28 |
| P2 | 7 | 17 | 25 | 35 | 51 |
| P3 | 9 | 21 | 30 | 41 | 62 |
| P4 | 6 | 13 | 21 | 29 | 48 |
| P5 | 4 | 3 | 7 | 11 | 15 |
| P6 | 4 | 5 | 7 | 13 | 16 |
| P7 | 6 | 9 | 18 | 27 | 42 |

TABLE 24
Percent Methionine Released and Bioavailability (After Curing)

| | % Released at | % Released at | % Released at | % Released at | % Released at | |
|---|---|---|---|---|---|---|
| # | 4 hours | 8 hours | 10 hours | 13 hours | 16 hours | Bioavailability |
| P1 | 3 | 9 | 44 | 86 | 95 | 86 |
| P2 | 9 | 21 | 56 | 94 | 98 | 78 |
| P3 | 12 | 26 | 56 | 96 | 99 | 73 |
| P4 | 8 | 18 | 41 | 85 | 96 | 78 |
| P5 | 2 | 5 | 44 | 84 | 89 | 84 |
| P6 | 2 | 5 | 39 | 87 | 93 | 88 |
| P7 | 5 | 13 | 36 | 74 | 88 | 75 |

The results show that bioavailability of capsules made with a pan coater was equally as high as the data obtained using a fluid bed. During coating, maintaining a batch temperature of at least 35° C. produces a high performing capsule prototype.

### Example 18: Methionine-Chitosan Acetate Pellets

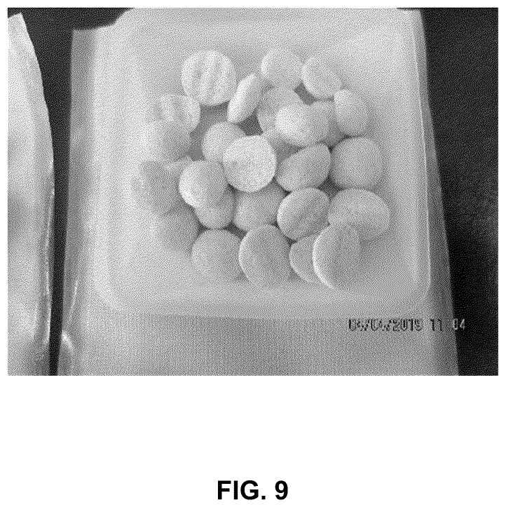

Pellets consisting of methionine and chitosan acetate were produced in the following manner. Distilled waterways added to a mixture of methionine and chitosan acetate powders and mixed vigorously with a spatula for 15 minutes. The material was scooped into a plastic bag. A small pinhole was made on the bag and the material was squeezed into HPLC vials or parchment paper and was dried overnight in a fume hood. The composition of the pellets is shown in Table 25. Dried pellets are shown in FIG. 9.

TABLE 25
Composition of Methionine-Chitosan Acetate pellets.

| | | Weight % | |
|---|---|---|---|
| Ingredient | Weight (g) | of composition | % Solids |
| Methionine powder (99%) | 38.44 | 54.7 | 54.2 |
| Chitosan acetate powder | 1.73 | 2.5 | 2.5 |
| Distilled water | 30.05 | 42.8 | — |
| Total | 70.22 | 100.0 | 56.7 |

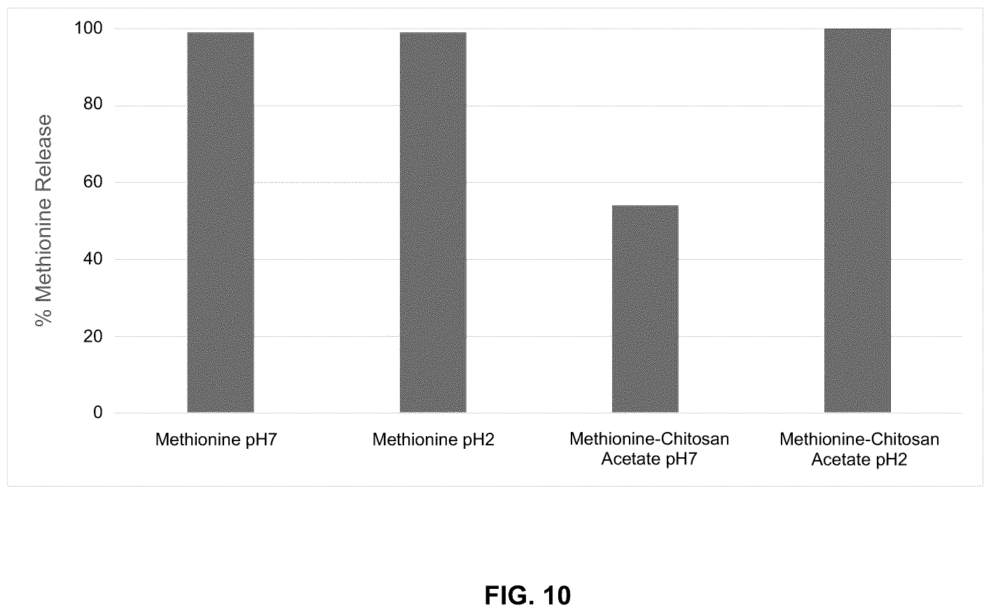

The pellets were then incubated in a pH 2.0 buffer and a pH 7.0 buffer for 2 hours at 39° C. For comparison, pellets consisting of methionine were also incubated in the buffer solutions. The results are shown in FIG. 10 and in Table 26 below. The pellets containing the chitosan acetate completely dissolved in the pH 2.0 buffer solution, but only partially dissolved in the pH 7.0 buffer solution. The pellets consisting only of methionine completely dissolved in both buffer solutions. Therefore, cores containing methionine and chitosan acetate would likely be even more resistant to rumen degradation when combined with an overcoat or microencapsulation process such as those described hereinabove, thereby improving the total bioavailability of the compositions even further.

TABLE 26
Methionine Pellet pH Test

| | | wt % |
|---|---|---|
| Sample | Test Solution | Release |
| Met Reference Standard (99.9% | 0.05M Phosphate Buffer, | 99 |
| purity) | pH 7.00 | |
| Met Reference Standard (99.9% | 0.01M Acid Buffer, | 99 |
| purity) | pH 2.00 | |
| Methionine-Chitosan Pellet Cores | 0.05M Phosphate Buffer, | 54 |
| (95.7 wt %) of total formulation | pH 7.00 | |
| Methionine-Chitosan Pellet Cores | 0.01M Acid Buffer, | 100 |
| (95.7 wt %) of total formulation | pH 2.00 | |

### Example 19: Lysine Rumen-Protected Compositions

Approximately 396 kg of lysine hydrochloride granules with a purity of 98.7% was introduced to a 9-barrel Extruder feeder line and mixed with about 44 kg of distilled water. The mixture was extruded at a temperature range of 75-125° C. The extruded cores had variable water content range of approximately 11 wt % of the total weight of lysine core formulation. After drying in a rotary oven at 70-75° C., the water content was reduced to about 1 wt %. The final extruded lysine core was approximately 99 wt % lysine hydrochloride.

The extruded lysine cores were then coated in a two-step process. A first coating mixture of about 8 kg composed of 7.2 kg of hydrogenated soybean oil and 0.8 kg of distilled palm oil was added to a jacketed vessel and melted at approximately 100° C. (range 90-115° C.). The extruded cores (22.7 kg) were added to a spray reactor and approximately 6.3 kg of the first coating mixture was applied by spraying the oil steadily to the cores at a batch temperature of approximately 50° C.

In the second step of the process, a second coating was added to the formulation according to the method described in Example 6. A suitable coating composition was prepared by dissolving 33 g of chitosan acetate in 967 g of warm distilled water (˜50° C.) to form 3.3% (wt %) solution. About 528 g of chitosan acetate solution was added to a mixture of 17 g of deoiled lecithin and 271 g of distilled water in a 2-liter vessel, and briefly homogenized to form a uniform dispersion. Approximately 183 g of fatty acid powder containing 50 wt % stearic acid and 50 wt % palmitic acid was gently added to the mixture followed by gentle overhead mixing to achieve wetting but still with visible large, clumped particles. The resultant mixture was homogenized with a high shear homogenizer at approximately 7500 rpm (Silverson 5L, rot-r—0“32” (8.00 mm), stat-r —1“38” (35.00 mm)) for about 5 minutes. The rumen-protected composition was smooth, free flowing, and sprayable in a fluid bed system at room temperature (e.g., about 25° C. to about 35° C.). The compositions of the final formulations are shown in Table 27.

TABLE 27
Lysine Rumen-Protected Compositions

| | Core | Coating 1 | Coating 2 | Final | | |
|---|---|---|---|---|---|---|
| | Ingredients | Ingredients | Ingredients | ingredients | Coating 1 | Coating 2 |
| | (wt % of | (wt % of coating 1 | (wt % of coating 2 | (wt % of final | wt % of final | wt % of final |
| # | Core alone) | alone; dry basis) | alone; dry basis) | composition) | composition | composition |
| L0 | Lysine: 99.0 | Hydrogenated | N/A | Lysine: 79.2 | 20.0 | N/A |
| | Water: 1.0 | soybean oil: 90.0 | | Water: 0.8 | | |
| | | Distilled palm | | Hydrogenated | | |
| | | oil: 10.0 | | soybean oil: 18.0 | | |
| | | | | Distilled palm | | |
| | | | | oil: 2.0 | | |
| L1 | Lysine: 99.0 | Hydrogenated | Lecithin: 7.9 | Lysine: 65.0 | 16.5 | 17.7 |
| | Water: 1.0 | soybean oil: 90.0 | Fatty Acid: 84.2 | Water: 0.7 | | |
| | | Distilled palm | Chitosan Acetate: 7.9 | Hydrogenated | | |
| | | oil: 10.0 | | soybean oil: 14.9 | | |
| | | | | Distilled palm | | |
| | | | | oil: 1.6 | | |
| | | | | Fatty acid: 14.9 | | |
| | | | | Chitosan acetate: 1.4 | | |
| | | | | Lecithin: 1.4 | | |
| L2 | Lysine: 99.0 | Hydrogenated | Lecithin: 8.3 | Lysine: 75.1 | 16.0 | 9.5 |
| | Water: 1.0 | soybean oil: 90.0 | Fatty Acid: 83.3 | Water: 0.7 | | |
| | | Distilled palm | Chitosan Acetate: 8.3 | Hydrogenated | | |
| | | oil: 10.0 | | soybean oil: 18.3 | | |
| | | | | Distilled palm | | |
| | | | | oil: 2.0 | | |
| | | | | Fatty acid: 8.0 | | |
| | | | | Chitosan acetate: 0.8 | | |
| | | | | Lecithin: 0.8 | | |
| L3 | Lysine: 99.0 | Hydrogenated | Lecithin: 8.2 | Lysine: 66.8 | 16.9 | 15.7 |
| | Water: 1.0 | soybean oil: 90.0 | Fatty Acid: 83.5 | Water: 0.7 | | |
| | | Distilled palm | Chitosan Acetate: 8.2 | Hydrogenated | | |
| | | oil: 10.0 | | soybean oil: 15.2 | | |
| | | | | Distilled palm | | |
| | | | | oil: 1.7 | | |
| | | | | Fatty acid: 13.2 | | |
| | | | | Chitosan acetate: 1.3 | | |
| | | | | Lecithin: 1.3 | | |
| L4 | Lysine: 99.0 | Hydrogenated | Lecithin: 8.1 | Lysine: 65.6 | 16.6 | 17.2 |
| | Water: 1.0 | soybean oil: 90.0 | Stearic Acid: 83.8 | Water: 0.7 | | |
| | | Distilled palm | Chitosan Acetate: 8.1 | Hydrogenated | | |
| | | oil: 10.0 | | soybean oil: 14.9 | | |
| | | | | Distilled palm | | |
| | | | | oil: 1.7 | | |
| | | | | Fatty acid: 14.5 | | |
| | | | | Chitosan acetate: 1.4 | | |
| | | | | Lecithin: 1.4 | | |
| L5 | Lysine: 99.0 | Hydrogenated | Lecithin: 7.9 | Lysine: 66.2 | 16.7 | 16.5 |
| | Water: 1.0 | soybean oil: 90.0 | Stearic Acid: 84.2 | Water: 0.7 | | |
| | | Distilled palm | Chitosan Acetate: 7.9 | Hydrogenated | | |
| | | oil: 10.0 | | soybean oil: 15.0 | | |
| | | | | Distilled palm | | |
| | | | | oil: 1.7 | | |
| | | | | Fatty acid: 13.8 | | |
| | | | | Chitosan acetate: 1.3 | | |
| | | | | Lecithin: 1.3 | | |

The compositions shown in Table 27 were then tested for rumen stability using the buffer/lipase assay as described herein. The results are described in Table 28. Rumen stability evaluation showed significant loss of L0 compared to the other compositions (L1L2, 1L3, 1L4, and L5). Although L2 had 2 coatings, it showed rapid release of the lysine compared to L1, L2, L3, L4, and L5. This was probably due a low 1^{st} coating level.

TABLE 28
Rumen Stability of Lysine Rumen-protected Compositions L0-L5

| | % Release | % Release | % Release | % Release |
|---|---|---|---|---|
| # | at 8 hours | at 12 hours | at 16 hours | at 24 hours |
| L0 | 86 | NA | NA | NA |
| L1 | 20 | 39 | 61 | 87 |
| L2 | 58 | 77 | 90 | 98 |
| L3 | 27 | 51 | 71 | 92 |
| L4 | 28 | 51 | 72 | 93 |
| L5 | 21 | 42 | 64 | 90 |

The compositions shown in Table 27 were then tested for total bioavailability using the bioavailability assay described in Example 8. The results are shown in Table 29. L2 was not tested for total bioavailability due to its relatively poor rumen stability. The control L0 showed a total bioavailability of about 18%, whereas L1, L2, L3, L4, and L5 all showed a total bioavailability of lysine from 67-79%, a significant increase as compared to the control.

TABLE 29
Total Bioavailability of Lysine Rumen-protected Compositions

| | | | | | % Bio- |
|---|---|---|---|---|---|
| | % Released | % Released | % Released | % Released | avail- |
| # | at 8 hours | at 10 hours | at 13 hours | at 16 hours | ability |
| L0 | 82 | 92 | 98 | 100 | 18 |
| L1 | 20 | 45 | 92 | 99 | 79 |
| L2 | NA | NA | NA | NA | NA |
| L3 | 28 | 64 | 96 | 98 | 70 |
| L4 | 29 | 65 | 94 | 96 | 67 |
| L5 | 23 | 54 | 92 | 95 | 72 |

In sum, the final coated lysine compositions (L1, L2, L3, L4, and L5) show significantly high improvement in total bioavailability compared to the control. More specifically, based on the weighting of lysine activity (the concentration of the lysine in the dosage form), the performance index (lysine activity×bioavailability) was about 14% (79.2%×18.0%), whereas the performance for L1, L3, L4, and L5 was 51%, 46%, 44%, and 47% respectively—more than three times higher than L0.

Additional rumen-protected lysine compositions were made according to the procedure outlined above. The content of these compositions is shown in Table 30.

TABLE 30
Lysine Rumen-protected Compositions

| | Core | Coating 1 | Coating 2 | Final | | |
|---|---|---|---|---|---|---|
| | Ingredients | Ingredients | Ingredients | ingredients | Coating 1 | Coating 2 |
| | (wt % of | (wt % coating | (wt % of coating 2 | (wt % of final | wt % of final | wt % of final |
| # | Core alone) | 1 alone) | alone; dry basis) | composition) | composition | composition |
| L6 | Lysine: 99.0 | Hydrogenated | N/A | Lysine: 77.2 | 22.0 | N/A |
| | Water: 1.0 | soybean oil: 100.0 | | Water: 0.8 | | |
| | | | | Hydrogenated | | |
| | | | | soybean oil: 22.0 | | |
| L7 | Lysine: 99.0 | Hydrogenated | Lecithin: 0.6 | Lysine: 71.2 | 20.3 | 7.8 |
| | Water: 1.0 | soybean oil: 100.0 | Fatty Acid: 6.6 | Water: 0.7 | | |
| | | | Chitosan Acetate: 0.6 | Hydrogenated | | |
| | | | | soybean oil: 20.3 | | |
| | | | | Fatty acid: 6.6 | | |
| | | | | Chitosan acetate: 0.6 | | |
| | | | | Lecithin: 0.6 | | |
| L8 | Lysine: 99.0 | Hydrogenated | Lecithin: 0.8 | Lysine: 69.2 | 19.7 | 10.4 |
| | Water: 1.0 | soybean oil: 100.0 | Fatty Acid: 8.7 | Water: 0.7 | | |
| | | | Chitosan Acetate: 0.8 | Hydrogenated | | |
| | | | | soybean oil: 19.7 | | |
| | | | | Fatty acid: 8.7 | | |
| | | | | Chitosan acetate: 0.8 | | |
| | | | | Lecithin: 0.8 | | |
| L9 | Lysine: 99.0 | Hydrogenated | Lecithin: 1.3 | Lysine: 65.0 | 18.5 | 15.8 |
| | Water: 1.0 | soybean oil: 100.0 | Fatty Acid: 13.3 | Water: 0.7 | | |
| | | | Chitosan Acetate: 1.3 | Hydrogenated | | |
| | | | | soybean oil: 18.5 | | |
| | | | | Fatty acid: 13.3 | | |
| | | | | Chitosan acetate: 1.3 | | |
| | | | | Lecithin: 1.3 | | |
| L10 | Lysine: 99.0 | Hydrogenated | Lecithin: 1.3 | Lysine: 64.6 | 18.4 | 16.4 |
| | Water: 1.0 | soybean oil: 100.0 | Fatty Acid: 13.7 | Water: 0.7 | | |
| | | | Chitosan Acetate: 1.3 | Hydrogenated | | |
| | | | | soybean oil: 18.4 | | |
| | | | | Fatty acid: 13.7 | | |
| | | | | Chitosan acetate: 1.3 | | |
| | | | | Lecithin: 1.3 | | |
| L11 | Lysine: 99.0 | Hydrogenated | Lecithin: 1.2 | Lysine: 65.7 | 18.7 | 14.9 |
| | Water: 1.0 | soybean oil: 100.0 | Fatty Acid: 12.5 | Water: 0.7 | | |
| | | | Chitosan Acetate: 1.2 | Hydrogenated | | |
| | | | | soybean oil: 18.7 | | |
| | | | | Fatty acid: 12.5 | | |
| | | | | Chitosan acetate: 1.2 | | |
| | | | | Lecithin: 1.2 | | |
| L12 | Lysine: 99.0 | Hydrogenated | Lecithin: 1.3 | Lysine: 65.0 | 18.5 | 15.8 |
| | Water: 1.0 | soybean oil: 100.0 | Fatty Acid: 13.3 | Water: 0.7 | | |
| | | | Chitosan Acetate: 1.3 | Hydrogenated | | |
| | | | | soybean oil: 18.5 | | |
| | | | | Fatty acid: 13.3 | | |
| | | | | Chitosan acetate: 1.3 | | |
| | | | | Lecithin: 1.3 | | |

The compositions in Table 30 were tested for Rumen Stability using the Buffer/Lipase assay. The results of the assay are shown in Table 31. L6 was used as the control composition, which released over 40% of the lysine within 8 hours.

TABLE 31
Rumen Stability of Lysine Rumen-protected Compositions L6-L12

| | % Release | % Release | % Release | % Release |
|---|---|---|---|---|
| # | at 8 hours | at 12 hours | at 16 hours | at 24 hours |
| L6 | 42 | NA | NA | NA |
| L7 | 35 | 53 | 66 | 82 |
| L8 | 22 | 37 | 50 | 69 |
| L9 | 14 | 30 | 43 | 66 |
| L10 | 18 | 32 | 49 | 72 |
| L11 | 13 | 25 | 37 | 60 |
| L12 | 14 | 26 | 38 | 64 |

The compositions shown in Table 30 were then tested for total bioavailability using the bioavailability assay described in Example 8. The results are shown in Table 32.

TABLE 32
Total Bioavailability of Lysine Rumen-protected Compositions

| | | | | | % Bio- |
|---|---|---|---|---|---|
| | % Released | % Released | % Released | % Released | avail- |
| # | at 8 hours | at 10 hours | at 13 hours | at 16 hours | ability |
| L6 | 44 | 52 | 67 | 74 | 30 |
| L7 | 33 | 52 | 77 | 86 | 53 |
| L8 | 23 | 41 | 63 | 75 | 52 |
| L9 | 15 | 36 | 71 | 76 | 61 |
| L10 | 15 | 34 | 68 | 70 | 55 |
| L11 | 11 | 26 | 56 | 61 | 50 |
| L12 | 12 | 29 | 60 | 64 | 52 |

The performance of L6 was about 23%, whereas the performance for L7-L12 was 38%, 36%, 40%, 35%, 33%, and 32% respectively. While in vitro rumen stability was generally acceptable (<40% release in 8 hours) for L1-L5 and L7-L12, total bioavailability was consistently related to the 1^{st} coating lipid composition; L1, L3, L4, and L5, which showed ranges from 67% to 79% bioavailability, while L7 to L12 showed bioavailability ranges from 50% to 61% bioavailability. The improved bioavailability and performance values of about 3.4× (L1, L3, L4, and L5) compared to L0, and about 1.6× (L7 to L12) compared to L6, is attributed to the mixed lipid composition of the 1^{st} coating containing distilled palm oil additive.

### Example 20: Rumen-Protected Histidine Hydrochloride Monohydrate Coated Compositions

Histidine hydrochloride monohydrate crystals were sieved on Ro Tap equipment using a stack of US Mesh sieves to obtain a fraction ranging from 850 microns (20 US Mesh) to 250 microns (60 US Mesh) particle size. The fraction was chosen to remove very fine particles, which present a challenge to optimal microencapsulation. The particle size distribution is shown in Table 33.

TABLE 33
Particle Size Distribution of Histidine
hydrochloride monohydrate crystals

| Us mesh | Opening size | Difference | Final |
|---|---|---|---|
| sieve # | (micron) | (g) | (wt % of total output) |
| 20 | 850 | 2.60 | 2.6 |
| 40 | 425 | 34.87 | 34.8 |
| 60 | 250 | 33.99 | 33.9 |
| 80 | 180 | 11.88 | 11.9 |
| 100 | 150 | 5.35 | 5.3 |
| 140 | 106 | 4.09 | 4.1 |
| Pan | <106 | 7.41 | 7.4 |
| OUTPUT | | 100.19 | 100.0 |

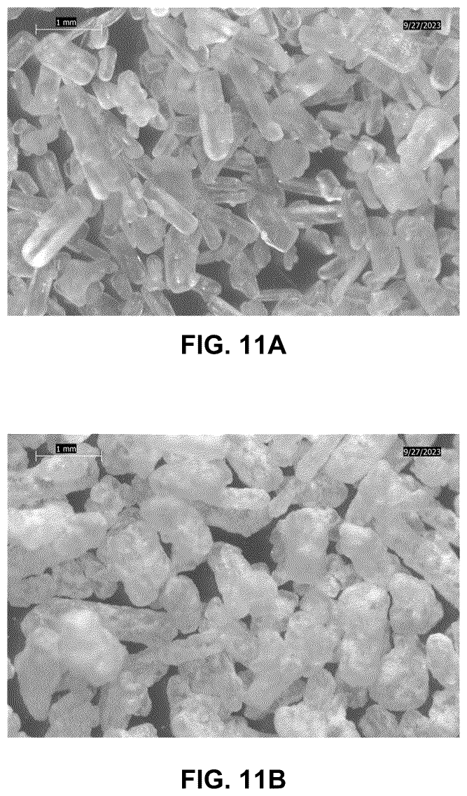

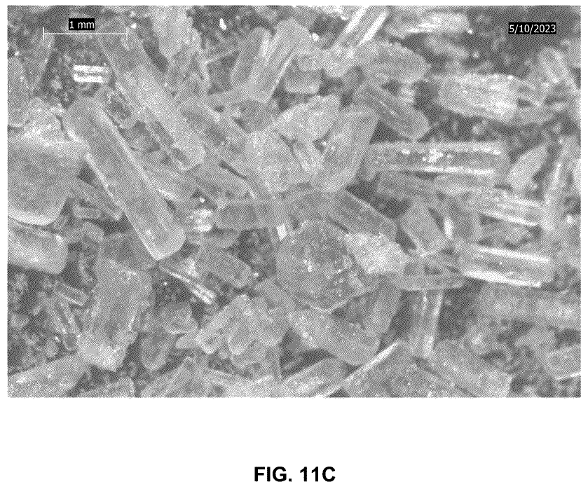

The crystals were microencapsulated using a reactor dispensing a hot lipid blend at about 80 degrees Celsius to achieve a coating level from 15 wt % to 55 wt %. The lipid blend included hydrogenated soybean oil, distilled palm oil, and monoglycerides. The lipid-coated particles are shown in FIG. 11A. The coated particles (ranging from 45 wt % to 85 wt % active histidine hydrochloride monohydrate) were further coated in a fluidized bed using an aqueous dispersion composition a mixture of fatty acid, chitosan acetate, and lecithin to a coating level of about 15 wt % to 25 wt % and dried. The fatty acid included a mixture of stearic acid and palmitic acid. The contents of the encapsulated histidine compositions are shown in Table 34. Images of the encapsulated crystals are shown in FIG. 11B. Images of the uncoated crystals are provided in FIG. 11C for comparison.

TABLE 34
Rumen-protected Histidine Compositions

| | | | | Coating 2 | | |
|---|---|---|---|---|---|---|
| | Core | Coating 1 | Coating 2 | Ingredients | Coating 1 | Coating 2 |
| | Ingredients | Ingredients (wt % | Ingredients (wt % | (wt % of coating 2 | wt % of final | wt % of final |
| | (wt % of Core) | coating 1 alone) | of coating 2 alone) | alone; dry basis) | composition | composition |
| H1 | Histidine | Lipid blend: 100.0 | N/A | Histidine | 27.8 | N/A |
| | hydrochloride | | | hydrochloride | | |
| | monohydrate: 99.7 | | | monohydrate: 72.0 | | |
| | Other: 0.3 | | | Lipid blend: 27.8 | | |
| | | | | Other: 0.2 | | |
| H2 | Histidine | Lipid blend: 100.0 | Lecithin: 1.4 | Histidine | 22.9 | 17.5 |
| | hydrochloride | | Fatty Acid: 14.7 | hydrochloride | | |
| | monohydrate: 99.7 | | Chitosan Acetate: 1.4 | monohydrate: 59.4 | | |
| | Other: 0.3 | | | Lipid blend: 22.9 | | |
| | | | | Fatty acid: 14.7 | | |
| | | | | Chitosan acetate: 1.4 | | |
| | | | | Lecithin: 1.4 | | |
| | | | | Other: 0.2 | | |
| H3 | Histidine | Lipid blend: 100.0 | Lecithin: 1.3 | Histidine | 23.2 | 16.5 |
| | hydrochloride | | Fatty Acid: 13.8 | hydrochloride | | |
| | monohydrate: 99.7 | | Chitosan Acetate: 1.3 | monohydrate: 60.1 | | |
| | Other: 0.3 | | | Lipid blend: 23.2 | | |
| | | | | Fatty acid: 13.8 | | |
| | | | | Chitosan acetate: 1.3 | | |
| | | | | Lecithin: 1.3 | | |
| | | | | Other: 0.2 | | |

The dry encapsulated histidine prototypes were evaluated for rumen stability and total bioavailability. The rumen stability was tested using the Buffer/Lipase assay. H1 served as the control. The results are shown in Table 35.

TABLE 35
Rumen Stability of Histidine Rumen-protected Compositions

| | % Release | % Release | % Release | % Release |
|---|---|---|---|---|
| # | at 8 hours | at 12 hours | at 16 hours | at 24 hours |
| H1 | 10 | 18 | 25 | 35 |
| H2 | 13 | 50 | 83 | 100 |
| H3 | 11 | 50 | 84 | 100 |

The total bioavailability of the compositions was tested using the Bioavailability assay described in Example 8. The results are described in Table 36.

TABLE 36
Total Bioavailability of Histidine Rumen-protected Compositions

| | | | | | % Bio- |
|---|---|---|---|---|---|
| | % Released | % Released | % Released | % Released | avail- |
| # | at 8 hours | at 10 hours | at 13 hours | at 16 hours | ability |
| H1 | 7 | 9 | 26 | 44 | 37 |
| H2 | 11 | 39 | 79 | 89 | 78 |
| H3 | 10 | 30 | 80 | 90 | 80 |

Although the histidine release at 8 hours in the rumen stability over all three compositions was similar, the total bioavailability of H2 and H3 were significantly improved as compared to H1.

### Example 21: Rumen-protected Choline Chloride Coated Compositions

850 kg of choline chlorine with a purity of 99% was mixed with 68 kg of hydrogenated soybean oil, 20 kg of calcium stearate, 17 kg of de-oiled lecithin, and 45 kg of silica. The mixture was extruded using an extruder with 10 sectors set at a temperature range of 70-99° C. The cores obtained had a concentration of 85 wt % choline chloride by weight of the core.

A coating mixture of about 8 kg composed of 7.2 kg of hydrogenated soybean oil and 0.8 kg of distilled palm oil was added to a jacketed vessel and melted at about 100° C. (range 90-115° C.). The coating mixture included 90% by weight of hydrogenated palm oil and 10% by weight distilled palmitic acid. The extruded cores (22.7 kg (50 lb) were added to a spray reactor and approximately 6.9 kg of the coating mixture was applied by spraying the coating mixture steadily to the cores at a batch temperature of about 50° C. A second coating was applied as described in Example 6. The content of the compositions is shown in Table 37.

TABLE 37
Rumen-protected Choline Chloride Compositions

| | | | Coating 2 | | | |
|---|---|---|---|---|---|---|
| | | | Ingredients | Final | | |
| | | Coating 1 | (wt % of | composition | | |
| | Core | Ingredients | coating 2 | ingredients | Coating 1 | Coating 2 |
| | Ingredients | (wt % coating 1 | alone; dry | (wt % of final | wt % of final | wt % of final |
| # | (wt % of Core) | alone) | basis) | composition) | composition | composition |
| C1 | Choline chloride - 85.0 | Hydrogenated | N/A | Choline chloride: 59.2 | 30.4 | N/A |
| | Hydrogenated | soybean oil: 90.0 | | Hydrogenated | | |
| | soybean oil - 6.8 | Distilled palm | | soybean oil: 32.1 | | |
| | Silica - 4.5 | oil: 10.0 | | Distilled palm | | |
| | Calcium stearate - 2.0 | | | oil: 3.0 | | |
| | Lecithin - 1.7 | | | Silica: 3.1 | | |
| | | | | Calcium stearate: 1.4 | | |
| | | | | Lecithin: 1.2 | | |
| C2 | Choline chloride - 85.0 | Hydrogenated | Lecithin: 1.2 | Choline chloride: 50.0 | 25.6 | 15.5 |
| | Hydrogenated | soybean oil: 90.0 | Fatty Acid: 13.0 | Hydrogenated | | |
| | soybean oil - 6.8 | Distilled palm | Chitosan Acetate: 1.2 | soybean oil: 27.0 | | |
| | Silica - 4.5 | oil: 10.0 | | Distilled palm | | |
| | Calcium stearate - 2.0 | | | oil: 2.6 | | |
| | Lecithin - 1.7 | | | Silica: 2.6 | | |
| | | | | Calcium stearate: 1.2 | | |
| | | | | Fatty acid: 13.0 | | |
| | | | | Chitosan acetate: 1.2 | | |
| | | | | Lecithin: 2.2 | | |

The compositions were tested for rumen stability and total bioavailability. The rumen stability was tested using the Buffer/Lipase assay. The results are shown in Table 38.

TABLE 38
Rumen Stability of Choline Compositions

| | % Release | % Release | % Release | % Release | % Release |
|---|---|---|---|---|---|
| # | at 4 hours | at 8 hours | at 12 hours | at 16 hours | at 24 hours |
| C1 | 12 | 33 | — | 68 | 83 |
| C2 | 27 | 49 | 72 | 86 | 95 |

The total bioavailability was tested using the bioavailability assay described in Example 8. The results are shown in Table 39.

TABLE 39
Total Bioavailability of Choline Compositions

| | % Released at | % Released at | % Released at | % Released at | % Released at | |
|---|---|---|---|---|---|---|
| # | 4 hours | 8 hours | 10 hours | 13 hours | 16 hours | % Bioavailability |
| C1 | 13 | 35 | 47 | 66 | 80 | 45 |
| C2 | 30 | 52 | 72 | 82 | 97 | 45 |

### Example 22: Mineral Mix Study

A mineral mix weighing about 2000 lbs (1000 kg) was prepared using the ingredients provided in Table 40. The vessel used for the mixing was equipped with a motorized paddle/ribbon mixer. Approximately 45 kg of material was mixed at a time. Approximately 1.0 kg of a coated rumen-protected composition of the present disclosure was mixed with 44.4 kg of mineral mix granules and blended together for 3 min. The final concentration of the coated rumen-protected composition was approximately 2.2 wt %. Each mixed material was sieved through US Mesh #8, #10, #14, and #16 to isolate the rumen-protected composition of the present disclosure from the mineral mix granules. Most of the rumen-protected compositions were collected on #14 mesh (1400 microns). The rumen-protected compositions were tested for mineral stability using Buffer/Lipase assay and compared to rumen-protected compositions that were not stored in a mineral mix, the results for which are shown in Table 41.

TABLE 40
Mineral Mix Composition

| Ingredient | Weight (lbs) |
|---|---|
| Limestone | 900 |
| Sodium Bicarbonate | 880 |
| Magnesium Oxide 54% | 200 |
| Soybean Oil | 20 |
| Total | 2000 |

TABLE 41
Buffer Lipase Assay Results

| | % Release at | % Release at | % Release at | % Release at | % Release at | % Release at |
|---|---|---|---|---|---|---|
| # | 0 hours | 4 hours | 8 hours | 12 hours | 16 hours | 24 hours |
| T1 Unmixed | 0 | 5 | 10 | 13 | 16 | 22 |
| T2 Unmixed | 0 | 4 | 8 | 11 | 14 | 20 |
| T1 Mixed | 0 | 4 | 8 | 12 | 14 | 21 |
| T2 Mixed | 0 | 5 | 9 | 13 | 16 | 23 |

The results show that there was no significant difference of the rumen stability after the compositions were stored in the mineral mix.

### Example 23: Rumen-protected Choline Compostions

Additional coated rumen-protected compositions including choline are provided below in Table 43. The compositions will be tested for rumen stability and total bioavailability according to the methods described herein.

The cores were produced using a 6-Sector temperature gradient extruder. Approximately 850 kg of Choline chlorine with a purity of 99% was mixed with 85 kg of a lipid blend comprising hydrogenated soybean oil (90%) and distilled palmitic acid (10%), 45 kg of silica, 20 kg of calcium stearate. This powdered blend was introduced to the extruder using a hopper, and the mixture was extruded using a 6-sector extruder set at different temperature gradients as shown below in Table 42. The cores were then coaled.

TABLE 42
Extruder Temperature Gradient

| Sector 1 | Sector 2 | Sector 3 | Sector 4 | Sector 5 | Sector 6 |
|---|---|---|---|---|---|
| 70° C. | 75° C. | 80° C. | 80° C. | 80° C. | 85° C. |

The cores thus obtained had a concentration of 85 wt % choline chloride. The cores were subsequently subjected to coating in a pan according to the methods described above.

TABLE 43
Rumen-protected Choline Compositions

| | | Coating 1 | Coating 2 | Final | | |
|---|---|---|---|---|---|---|
| | Core | Ingredients | Ingredients | Ingredients | Coating 1 | Coating 2 |
| | Ingredients | (wt % of coating 1 | (wt % of coating 2 | (wt % of final | wt % of final | wt % of final |
| # | (wt % of Core alone) | alone; dry basis) | alone; dry basis) | composition) | composition | composition |
| C3 | Choline chloride - 85.0 | Hydrogenated | NA | Choline chloride - 72.3 | 15 | NA |
| | Hydrogenated | soybean oil - 100.0 | | Hydrogenated | | |
| | soybean oil - 8.5 | | | soybean oil - 22.2 | | |
| | Silica - 4.5 | | | Silica - 3.8 | | |
| | Calcium stearate - 2.0 | | | Calcium stearate - 1.7 | | |
| C4 | Choline chloride - 85.0 | Hydrogenated | NA | Choline chloride - 68.0 | 20 | NA |
| | Hydrogenated | soybean oil - 100.0 | | Hydrogenated | | |
| | soybean oil - 8.5 | | | soybean oil - 26.8 | | |
| | Silica - 4.5 | | | Silica - 3.6 | | |
| | Calcium stearate - 2.0 | | | Calcium stearate - 1.6 | | |
| C5 | Choline chloride - 85.0 | Hydrogenated | NA | Choline chloride - 63.8 | 25 | NA |
| | Hydrogenated | soybean oil - 100.0 | | Hydrogenated | | |
| | soybean oil - 8.5 | | | soybean oil - 31.4 | | |
| | Silica - 4.5 | | | Silica - 3.4 | | |
| | Calcium stearate - 2.0 | | | Calcium stearate - 1.5 | | |
| C6 | Choline chloride - 85.0 | Hydrogenated | NA | Choline chloride - 59.5 | 30 | NA |
| | Hydrogenated | soybean oil - 100.0 | | Hydrogenated | | |
| | soybean oil - 8.5 | | | soybean oil - 36.0 | | |
| | Silica - 4.5 | | | Silica - 3.2 | | |
| | Calcium stearate - 2.0 | | | Calcium stearate - 1.4 | | |
| C7 | Choline chloride - 85.0 | Hydrogenated | Fatty acids - 10.1 | Choline chloride - 63.6 | 15 | 12 |
| | Hydrogenated | soybean oil - 100.0 | Chitosan acetate - 1.0 | Hydrogenated | | |
| | soybean oil - 8.5 | | Lecithin - 1.0 | soybean oil - 19.6 | | |
| | Silica - 4.5 | | | Silica - 3.4 | | |
| | Calcium stearate - 2.0 | | | Calcium stearate - 1.5 | | |
| | | | | Fatty acids - 10.1 | | |
| | | | | Chitosan acetate - 1.0 | | |
| | | | | Lecithin - 1.0 | | |
| C8 | Choline chloride - 85.0 | Hydrogenated | Fatty acids - 10.1 | Choline chloride - 59.8 | 20 | 12 |
| | Hydrogenated | soybean oil - 100.0 | Chitosan acetate - 1.0 | Hydrogenated | | |
| | soybean oil - 8.5 | | Lecithin - 1.0 | soybean oil - 23.6 | | |
| | Silica - 4.5 | | | Silica - 3.2 | | |
| | Calcium stearate - 2.0 | | | Calcium stearate - 1.4 | | |
| | | | | Fatty acids - 10.1 | | |
| | | | | Chitosan acetate - 1.0 | | |
| | | | | Lecithin - 1.0 | | |
| C9 | Choline chloride - 85.0 | Hydrogenated | Fatty acids - 10.1 | Choline chloride - 56.1 | 25 | 12 |
| | Hydrogenated | soybean oil - 100.0 | Chitosan acetate - 1.0 | Hydrogenated | | |
| | soybean oil - 8.5 | | Lecithin - 1.0 | soybean oil - 27.6 | | |
| | Silica - 4.5 | | | Silica - 3.0 | | |
| | Calcium stearate - 2.0 | | | Calcium stearate - 1.3 | | |
| | | | | Fatty acids - 10.1 | | |
| | | | | Chitosan acetate - 1.0 | | |
| | | | | Lecithin - 1.0 | | |
| C10 | Choline chloride - 85.0 | Hydrogenated | Fatty acids - 10.1 | Choline chloride - 52.4 | 30 | 12 |
| | Hydrogenated | soybean oil - 100.0 | Chitosan acetate - 1.0 | Hydrogenated | | |
| | soybean oil - 8.5 | | Lecithin - 1.0 | soybean oil - 31.6 | | |
| | Silica - 4.5 | | | Silica - 2.8 | | |
| | Calcium stearate - 2.0 | | | Calcium stearate - 1.2 | | |
| | | | | Fatty acids - 10.1 | | |
| | | | | Chitosan acetate - 1.0 | | |
| | | | | Lecithin - 1.0 | | |
| C11 | Choline chloride - 85.0 | Hydrogenated | Fatty acids - 11.8 | Choline chloride - 62.1 | 15 | 14 |
| | Hydrogenated | soybean oil - 100.0 | Chitosan acetate - 1.1 | Hydrogenated | | |
| | soybean oil - 8.5 | | Lecithin - 1.1 | soybean oil - 19.1 | | |
| | Silica - 4.5 | | | Silica - 3.3 | | |
| | Calcium stearate - 2.0 | | | Calcium stearate - 1.5 | | |
| | | | | Fatty acids - 11.8 | | |
| | | | | Chitosan acetate - 1.1 | | |
| | | | | Lecithin - 1.1 | | |
| C12 | Choline chloride - 85.0 | Hydrogenated | Fatty acids - 11.8 | Choline chloride - 58.5 | 20 | 14 |
| | Hydrogenated | soybean oil - 100.0 | Chitosan acetate - 1.1 | Hydrogenated | | |
| | soybean oil - 8.5 | | Lecithin - 1.1 | soybean oil - 23.0 | | |
| | Silica - 4.5 | | | Silica - 3.1 | | |
| | Calcium stearate - 2.0 | | | Calcium stearate - 1.4 | | |
| | | | | Fatty acids - 11.8 | | |
| | | | | Chitosan acetate - 1.1 | | |
| | | | | Lecithin - 1.1 | | |
| C13 | Choline chloride - 85.0 | Hydrogenated | Fatty acids - 11.8 | Choline chloride - 54.8 | 25 | 14 |
| | Hydrogenated | soybean oil - 100.0 | Chitosan acetate - 1.1 | Hydrogenated | | |
| | soybean oil - 8.5 | | Lecithin - 1.1 | soybean oil - 27.0 | | |
| | Silica - 4.5 | | | Silica - 2.9 | | |
| | Calcium stearate - 2.0 | | | Calcium stearate - 1.3 | | |
| | | | | Fatty acids - 11.8 | | |
| | | | | Chitosan acetate - 1.1 | | |
| | | | | Lecithin - 1.1 | | |
| C14 | Choline chloride - 85.0 | Hydrogenated | Fatty acids - 11.8 | Choline chloride - 51.2 | 30 | 14 |
| | Hydrogenated | soybean oil - 100.0 | Chitosan acetate - 1.1 | Hydrogenated | | |
| | soybean oil - 8.5 | | Lecithin - 1.1 | soybean oil - 30.9 | | |
| | Silica - 4.5 | | | Silica - 2.7 | | |
| | Calcium stearate - 2.0 | | | Calcium stearate - 1.2 | | |
| | | | | Fatty acids - 11.8 | | |
| | | | | Chitosan acetate - 1.1 | | |
| | | | | Lecithin - 1.1 | | |
| C15 | Choline chloride - 85.0 | Hydrogenated | Fatty acids - 13.4 | Choline chloride - 60.7 | 15 | 16 |
| | Hydrogenated | soybean oil - 100.0 | Chitosan acetate - 1.3 | Hydrogenated | | |
| | soybean oil - 8.5 | | Lecithin - 1.3 | soybean oil - 18.7 | | |
| | Silica - 4.5 | | | Silica - 3.2 | | |
| | Calcium stearate - 2.0 | | | Calcium stearate - 1.4 | | |
| | | | | Fatty acids - 13.4 | | |
| | | | | Chitosan acetate - 1.3 | | |
| | | | | Lecithin - 1.3 | | |
| C16 | Choline chloride - 85.0 | Hydrogenated | Fatty acids - 13.4 | Choline chloride - 57.1 | 20 | 16 |
| | Hydrogenated | soybean oil - 100.0 | Chitosan acetate - 1.3 | Hydrogenated | | |
| | soybean oil - 8.5 | | Lecithin - 1.3 | soybean oil - 22.5 | | |
| | Silica - 4.5 | | | Silica - 3.0 | | |
| | Calcium stearate - 2.0 | | | Calcium stearate - 1.3 | | |
| | | | | Fatty acids - 13.4 | | |
| | | | | Chitosan acetate - 1.3 | | |
| | | | | Lecithin - 1.3 | | |
| C17 | Choline chloride - 85.0 | Hydrogenated | Fatty acids - 13.4 | Choline chloride - 53.6 | 25 | 16 |
| | Hydrogenated | soybean oil - 100.0 | Chitosan acetate - 1.3 | Hydrogenated | | |
| | soybean oil - 8.5 | | Lecithin - 1.3 | soybean oil - 26.4 | | |
| | Silica - 4.5 | | | Silica - 2.8 | | |
| | Calcium stearate - 2.0 | | | Calcium stearate - 1.3 | | |
| | | | | Fatty acids - 13.4 | | |
| | | | | Chitosan acetate - 1.3 | | |
| | | | | Lecithin - 1.3 | | |
| C18 | Choline chloride - 85.0 | Hydrogenated | Fatty acids - 13.4 | Choline chloride - 50.0 | 30 | 16 |
| | Hydrogenated | soybean oil - 100.0 | Chitosan acetate - 1.3 | Hydrogenated | | |
| | soybean oil - 8.5 | | Lecithin - 1.3 | soybean oil - 30.2 | | |
| | Silica - 4.5 | | | Silica - 2.6 | | |
| | Calcium stearate - 2.0 | | | Calcium stearate - 1.2 | | |
| | | | | Fatty acids - 13.4 | | |
| | | | | Chitosan acetate - 1.3 | | |
| | | | | Lecithin - 1.3 | | |

### Example 24: Rumen-protected Lysine Compositions

Additional coated rumen-protected compositions including lysine are provided below in Table 44.

Lysine cores L18-L23 were made via extrusion. Approximately 850 kg of lysine hydrochloride with a purity of 99% were mixed with 150 kg of hydrogenated soybean oil.

The compositions will be tested for rumen stability and total bioavailability according to the methods described herein.

TABLE 44
Rumen-protected Lysine Compositions

| | Core | Coating | Final | Coating wt % |
|---|---|---|---|---|
| | Ingredients (wt % | Ingredients (wt % | Ingredients (wt % | of final |
| # | of Core alone) | of coating alone) | of final composition) | composition |
| L15 | Lysine hydrochloride - 99.0 | Fatty acids - 13.4 | Lysine hydrochloride - 83.2 | 16 |
| | Water - 1.0 | Chitosan Acetate - 1.3 | Water - 0.8 | |
| | | Lecithin - 1.3 | Fatty acids - 13.4 | |
| | | | Chitosan Acetate - 1.3 | |
| | | | Lecithin - 1.3 | |
| L16 | Lysine hydrochloride - 99.0 | Fatty acids - 15.1 | Lysine hydrochloride - 81.2 | 18 |
| | Water - 1.0 | Chitosan Acetate - 1.4 | Water - 0.8 | |
| | | Lecithin - 1.4 | Fatty acids - 15.1 | |
| | | | Chitosan Acetate - 1.4 | |
| | | | Lecith-n - 1.4 | |
| L17 | Lysine hydrochloride - 99.0 | Fatty acids - 16.8 | Lysine hydrochloride - 79.2 | 20 |
| | Water - 1.0 | Chitosan Acetate - 1.6 | Water - 0.8 | |
| | | Lecithin - 1.6 | Fatty acids - 16.8 | |
| | | | Chitosan Acetate - 1.6 | |
| | | | Lecithin - 1.6 | |
| L18 | Lysine hydrochloride - 85.0 | Fatty acids - 11.8 | Lysine hydrochloride - 73.1 | 14 |
| | Hydrogenated soybean | Chitosan Acetate - 1.1 | Hydrogenated soybean oil - 12.9 | |
| | oil - 15.0 | Lecithin - 1.1 | Fatty acids - 11.8 | |
| | | | Chitosan Acetate - 1.1 | |
| | | | Lecithin - 1.1 | |
| L19 | Lysine hydrochloride - 85.0 | Fatty acids - 13.4 | Lysine hydrochloride - 71.4 | 16 |
| | Hydrogenated soybean | Chitosan Acetate - 1.3 | Hydrogenated soybean oil - 12.6 | |
| | oil - 15.0 | Lecithin - 1.3 | Fatty acids - 13.4 | |
| | | | Chitosan Acetate - 1.3 | |
| | | | Lecithin - 1.3 | |
| L20 | Lysine hydrochloride - 85.0 | Fatty acids - 15.1 | Lysine hydrochloride - 69.7 | 20 |
| | Hydrogenated soybean | Chitosan Acetate - 1.4 | Hydrogenated soybean oil - 12.3 | |
| | oil - 15.0 | Lecithin - 1.4 | Fatty acids - 15.1 | |
| | | | Chitosan Acetate - 1.4 | |
| | | | Lecithin - 1.4 | |
| L21 | Lysine hydrochloride - 80.0 | Fatty acids - 11.8 | Lysine hydrochloride - 68.8 | 14 |
| | Hydrogenated soybean | Chitosan Acetate - 1.1 | Hydrogenated soybean oil - 15.5 | |
| | oil - 18.0 | Lecithin - 1.1 | Palm oil - 1.7 | |
| | Palm oil - 2.0 | | Fatty acids - 15.1 | |
| | | | Chitosan Acetate - 1.4 | |
| | | | Lecithin - 1.4 | |
| L22 | Lysine hydrochloride - 80.0 | Fatty acids - 13.4 | Lysine hydrochloride - 67.2 | 16 |
| | Hydrogenated soybean | Chitosan Acetate - 1.3 | Hydrogenated soybean oil - 15.1 | |
| | oil - 18.0 | Lecithin - 1.3 | Palm oil - 1.7 | |
| | Palm oil - 2.0 | | Fatty acids - 13.4 | |
| | | | Chitosan Acetate - 1.3 | |
| | | | Lecithin - 1.3 | |
| L23 | Lysine hydrochloride - 80.0 | Fatty acids - 15.1 | Lysine hydrochloride - 65.6 | 18 |
| | Hydrogenated soybean | Chitosan Acetate - 1.4 | Hydrogenated soybean oil - 14.8 | |
| | oil - 18.0 | Lecithin - 1.4 | Palm oil - 1.6 | |
| | Palm oil - 2.0 | | Fatty acids - 15.1 | |
| | | | Chitosan Acetate - 1.4 | |
| | | | Lecithin - 1.4 | |

### Example 25: Rumen-protected Histidine Compostions

Additional coated rumen-protected compositions including histidine are provided below in Table 45. The cores will be produced using an extruder, preferably a 6-sector configuration extruder set at different temperature gradients as described in Example 1. Approximately 850 kg of histidine hydrochloride monohydrate with a purity of 99.7% will be mixed with 8.5 kg of a lipid blend composed of hydrogenated soybean oil (90%) and distilled palm oil (10%), 45 kg of silica, and 20 kg of calcium stearate. The cores would be subjected to coating in a pan or air-assisted fluid bed using the coating compositions and methods described herein.

TABLE 45
Rumen Protected Histidine Compositions

| | Core | Coating | Final | Coating wt % |
|---|---|---|---|---|
| | Ingredients (wt % | Ingredients (wt % | Ingredients (wt % | of final |
| # | of Core alone) | coating alone) | of final composition) | composition |
| H4 | Histidine hydrochloride | Fatty acids - 13.4 | Histidine hydrochloride | 16 |
| | monohydrate - 85.0 | Chitosan Acetate - 1.3 | monohydrate - 71.4 | |
| | Hydrogenated soybean | Lecithin - 1.3 | Hydrogenated soybean | |
| | oil - 8.5 | | oil - 7.1 | |
| | Silica - 4.5 | | Silica - 3.8 | |
| | Calcium stearate - 2.0 | | Calcium stearate - 1.7 | |
| | | | Fatty acids - 13.4 | |
| | | | Chitosan Acetate - 1.3 | |
| | | | Lecithin - 1.3 | |

What is claimed is:

1. A rumen-protected composition comprising a bioactive core comprising a physiologically active ingredient, the bioactive core surrounded by at least one coating layer, the at least one coating layer comprising:
(a) a chitosan organic salt;
(b) an emulsifier; and
(c) a fatty acid source,
wherein the coating is present in an amount of at least 10% by dry weight of the composition and the ratio of chitosan organic salt to emulsifier in the coating layer is less than about 2.5:1 by weight.

2. The rumen-protected composition of claim 1, wherein the physiologically active ingredient is selected from the group consisting of methionine, lysine, histidine, choline, and any combination thereof.

3. The rumen-protected composition of claim 1, wherein the physiologically active ingredient has a total bioavailability of at least about 70%.

4. The rumen-protected composition of claim 1, wherein the physiologically active ingredient has a rumen bypass rate of at least about 70%.

5. The rumen-protected composition of claim 1, wherein the chitosan organic salt is selected from the group consisting of chitosan acetate, chitosan glutamate, chitosan thiaminate, chitosan aspartate, chitosan nicotinate, and chitosan citrate.

6. The rumen-protected composition of claim 1, wherein the chitosan organic salt is chitosan acetate.

7. The rumen-protected composition of claim 1, wherein the emulsifier is selected from the group consisting of lecithin, monoglycerides, diglycerides, polyglycerol esters, propylene glycol esters, gums, waxes, phosphates, cellulose derivatives, and combinations thereof.

8. The rumen-protected composition of claim 1, wherein the emulsifier is lecithin.

9. The rumen-protected composition of claim 1, wherein the bioactive core comprises the physiologically active ingredient in an amount from about 50% to about 80% by weight of the rumen-protected composition, and the at least one coating layer comprises:
the chitosan organic salt in an amount from about 2% to about 3% by weight of the rumen-protected composition,
the emulsifier in an amount from about 0.5% to about 3% by weight of the rumen-protected composition, and
the fatty acid source in an amount from about 10% to about 20% by weight of the rumen-protected composition.

10. The rumen-protected composition of claim 9, wherein the physiologically active ingredient has a total bioavailability of at least 50% and the rumen bypass rate of the physiologically active ingredient is at least about 50%.

11. The rumen-protected composition of claim 1, wherein the at least one coating layer does not include a fat.

12. The rumen-protected composition of claim 1, wherein the ratio of the chitosan organic salt to the emulsifier in the coating layer is less than about 2:1 by weight.

13. The rumen-protected composition of claim 1, further comprising a sub-coating disposed between the bioactive core and the at least one coating layer.

14. The rumen-protected composition of claim 13, wherein the sub-coating comprises an oil selected from the group consisting of soybean oil, palm oil, rapeseed oil, cotton oil, linseed oil, castor oil, and other hydrophobic plant-based oils known in the art and combinations thereof.

15. The rumen-protected composition of claim 2, wherein the physiologically active ingredient is methionine.

16. The rumen-protected composition of claim 15, wherein the methionine is present in the rumen-protected composition in an amount from about 30% to about 85% by weight of the rumen-protected composition.

17. The rumen-protected composition of claim 1, wherein the bioactive core further comprises a fatty acid source.

18. The rumen-protected composition of claim 1, wherein the composition further comprises an excipient.

19. A rumen-protected composition comprising a bioactive core comprising a physiologically active ingredient selected from the group consisting of methionine, lysine, histidine, and any combination thereof; the core surrounded by at least one coating layer, the coating layer comprising:
(a) chitosan acetate;
(b) lecithin; and
(c) a fatty acid source, wherein the coating is present in an amount of at least 10% by dry weight of the composition and the ratio of chitosan acetate to lecithin in the coating layer is less than about 2.5:1 by weight.

20. A method of making a rumen-protected composition, the method comprising coating a bioactive core comprising a physiologically active ingredient with a coating composition, wherein:
the physiologically active ingredient is selected from the group consisting of methionine, lysine, histidine, choline, and any combination thereof; and
the coating composition comprises a chitosan organic salt, an emulsifier, and a fatty acid source.
